(19) United States
(12) Patent Application Publication
(10) Pub. No.: US 2016/0322577 A1
(43) Pub. Date: Nov. 3, 2016

(54) COMPOUND AND ORGANIC LIGHT-EMITTING DEVICE INCLUDING THE SAME

(71) Applicant: SAMSUNG DISPLAY CO., LTD., Yongin-si (KR)

(72) Inventors: Sanghyun Han, Yongin-si (KR); Sooyon Kim, Yongin-si (KR); Youngkook Kim, Yongin-si (KR); Seokhwan Hwang, Yongin-si (KR)

(21) Appl. No.: 15/004,490

(22) Filed: Jan. 22, 2016

(30) Foreign Application Priority Data
Apr. 28, 2015 (KR) 10-2015-0059635

(51) Int. Cl. H01L 51/00; C09K 11/06; C07D 409/12; C07D 409/14; C07F 7/08; C07D 307/93; H01L 27/32; C07D 333/78

(52) U.S. Cl. CPC H01L 51/0061; H01L 27/3248; H01L 51/0094; C09K 11/06; C07D 333/78; C07D 409/14; C07F 7/0812; C07D 307/93; C07D 409/12; H01L 51/5012

(57) ABSTRACT

A compound represented by Formula 1 and an organic light-emitting device including the compound are disclosed, wherein descriptions of Formula 1 are provided in the detailed description in the present specification. An organic layer of the organic light-emitting device may include the compound represented by Formula 1. The compound represented by Formula 1 may be included in an emission layer of the organic layer.

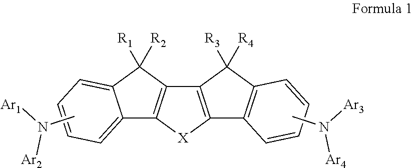

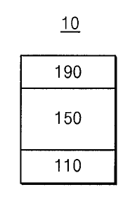

### CROSS-REFERENCE TO RELATED APPLICATION

This application claims priority to and the benefit of Korean Patent Application No. 10-2015-0059635, filed on Apr. 28, 2015, in the Korean Intellectual Property Office, the entire content of which is incorporated herein by reference.

### BACKGROUND

1. Field

One or more example embodiments relate to a compound and an organic light-emitting device including the same.

2. Description of the Related Art

Organic light-emitting devices (OLEDs) are self-emission devices that have wide viewing angles, high contrast ratios, and short response times. OLEDs also exhibit excellent brightness, driving voltage, and response speed characteristics, and produce multicolored images.

An OLED may include a first electrode disposed on a substrate, and a hole transport region, an emission layer, an electron transport region, and a second electrode, which are sequentially disposed on the first electrode. Holes provided from the first electrode move toward the emission layer through the hole transport region, and electrons provided from the second electrode move toward the emission layer through the electron transport region. Carriers, such as holes and electrons, are recombined in the emission layer to produce excitons. These excitons change from an excited state to a ground state, thereby generating light.

### SUMMARY

One or more example embodiments include a blue fluorescent dopant compound having improved high efficiency, low driving voltage, high luminance, and long lifespan characteristics, and an organic light-emitting device including the blue fluorescent dopant compound.

Additional aspects of embodiments will be set forth in part in the description which follows and, in part, will be apparent from the description, or may be learned by practice of the presented embodiments.

According to one or more example embodiments, there is provided a compound represented by Formula 1:

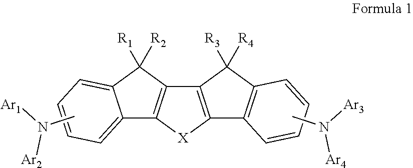

In Formula 1,

R₁ to R₄ may each be independently selected from a hydrogen, a deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

Ar₁ to Ar₄ may each be independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

X may be selected from oxygen (O), sulfur (S) and selenium (Se); and

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group may be selected from:

a deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₆), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—Si(Q₃₁)(Q₃₂)(Q₃₃),

wherein Q₁₁ to Q₁₇, Q₂₁ to Q₂₇, and Q₃₁ to Q₃₃ may be each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

According to one or more example embodiments, there is provided an organic light-emitting device including a first electrode; a second electrode facing the first electrode; and an organic layer disposed between the first electrode and the second electrode and including an emission layer, wherein the organic layer includes the compound represented by Formula 1.

According to one or more example embodiments, there is provided a flat display apparatus including the organic light-emitting device of which the first electrode is electrically coupled to source and drain electrodes of a thin film transistor.

### BRIEF DESCRIPTION OF THE DRAWINGS

These and/or other aspects will become apparent and more readily appreciated from the following description of example embodiments, taken in conjunction with the accompanying drawing which is a schematic view of an organic light-emitting device according to an embodiment.

### DETAILED DESCRIPTION

Reference will now be made in more detail to example embodiments, examples of which are illustrated in the accompanying drawing. In this regard, the present example embodiments may have different forms and should not be construed as being limited to the descriptions set forth herein. Accordingly, the example embodiments are merely described below, by referring to the accompanying drawing, to explain aspects of embodiments of the present description. As used herein, the term “and/or” includes any and all combinations of one or more of the associated listed items. Expressions such as “at least one of”, “when preceding a list of elements, modify the entire list of elements and do not modify the individual elements of the list. Further, the use of “may” when describing embodiments of the present invention refers to one or more embodiments of the present invention.”

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the accompanying drawing. For example, if the device in the figures is turned over, elements described as “below” or “beneath” or “under” other elements or features would then be oriented “above” the other elements or features. Thus, the example terms “below” and “under” can encompass both an orientation of above and below. For example, in the context of the present disclosure, an emission layer may be above or below a first electrode. Further, the device may be otherwise oriented (e.g., rotated 90 degrees or at other orientations) and the spatially relative descriptors used herein should be interpreted accordingly.

There is provided a compound represented by Formula 1:

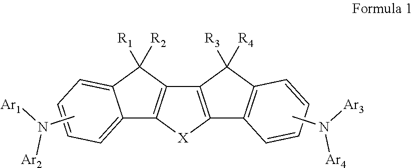

In Formula 1,

R₁ to R₄ may each be independently selected from a hydrogen, a deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

Ar₁ to Ar₄ may each be independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;

X may be selected from oxygen (O), sulfur (S), and selenium (Se); and

at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group may be selected from:

a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—Si(Q₃₁)(Q₃₂)(Q₃₃),

wherein Q₁₁ to Q₁₇, Q₂₁ to Q₂₇, and Q₃₁ to Q₃₃ may each be independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Regarding a blue light-emitting material of the related art, a blue light-emitting compound having a core structure of diphenyl anthracene and including an aryl group substituted at a terminal (e.g., at a terminal end), and an organic light-emitting device including the blue light-emitting compound have been utilized. However, such an organic light-emitting device fails to exhibit sufficient or suitable emission efficiency and brightness.

A compound including a substituted pyrene-based moiety and an organic light-emitting device including the pyrene-based compound have been utilized in the art. However, due to low color purity of blue, such an organic light-emitting device has a difficulty in implementing deep blue color, and thus there are problems in implementing multi-colored, full color display.

To solve the problems above, embodiments of the present disclosure include a novel compound and an organic light-emitting including the novel compound.

The novel compound of the present disclosure has excellent electric characteristics, high electron transporting capability, and light-emitting capability. In some embodiments, the novel compound may have a high glass transition temperature and prevent from crystallization (e.g., the crystallization temperature of the compound may be increased), and thus may be suitable for fluorescent and phosphorescent devices of all colors including red, green, blue, and white. The novel compound may be also used to manufacture an organic light-emitting device that has high efficiency, low voltage, high brightness, and long lifespan characteristics.

The substituents of Formula 1 will be described in more detail.

According to an example embodiment, in Formula 1, R₁ to R₄ may each be independently a substituted or unsubstituted C₁-C₆₀ alkyl group.

According to an example embodiment, in Formula 1, Ar₁ to Ar₄ may each be independently selected from groups represented by Formulae 2a to 2d:

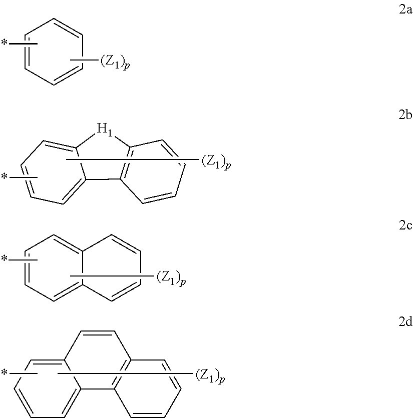

In Formulae 2a to 2d,

H₁ may be selected from CR₁₁R₁₂, O, and S;

R₁₁, R₁₂, and Z₁ may each be independently selected from a hydrogen, a deuterium, a halogen group, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₁)(Q₃₂)(Q₃₃), where Q₃₁ to Q₃₃ may be the same as described with respect to Formula 1;

in the case of a plurality of Z₁s, each of the Z₁s may be identical to or different from each other (e.g., each of the Z₁s is identical to or different from the others of the Z₁ s);

p may be an integer selected from 1 to 9; and

* may indicate a binding site.

According to an example embodiment, in Formula 1, X may be O or S.

According to an example embodiment, the compound represented by Formula 1 may be represented by one of Formulae 2 to 4:

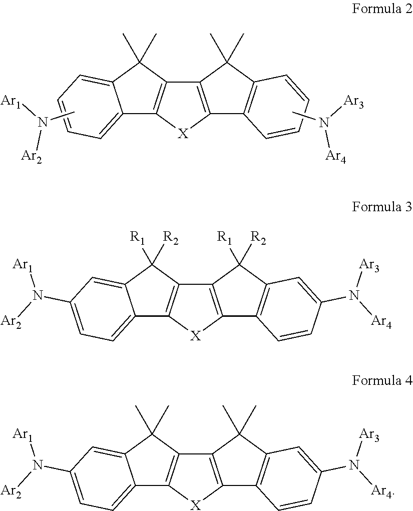

The substituents of Formulae 2 to 4 may be defined as described above. According to an example embodiment, the compound represented by Formula 1 may be one of the compounds 1-128 below:

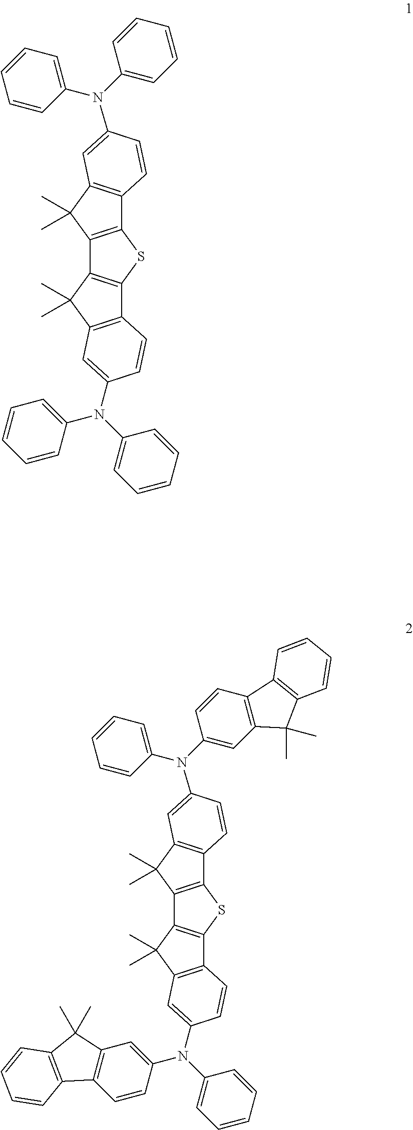 [SMILES: CC1(C)c2cc(N(c3ccccc3)c3ccccc3)ccc2-c2sc3c(c21)C(C)(C)c1cc(N(c2ccccc2)c2ccccc2)ccc1-3 ; CC1(C)c2ccccc2-c2ccc(N(c3ccccc3)c3ccc4c(c3)C(C)(C)c3c-4sc4c3C(C)(C)c3cc(N(c5ccccc5)c5ccc6c(c5)C(C)(C)c5ccccc5-6)ccc3-4)cc21]

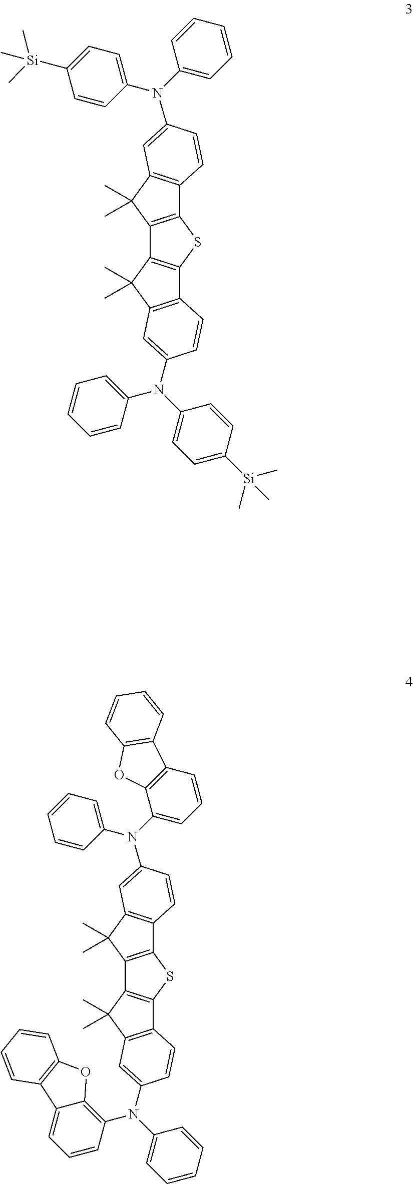

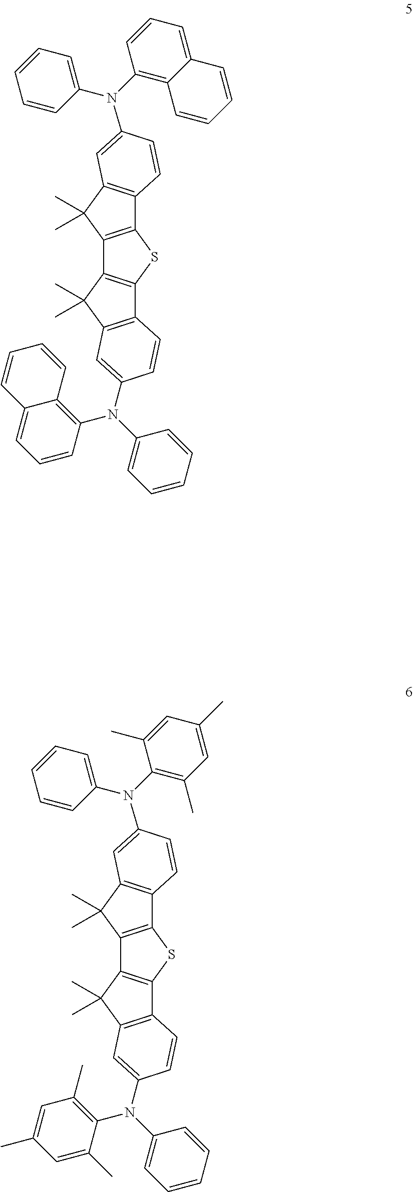

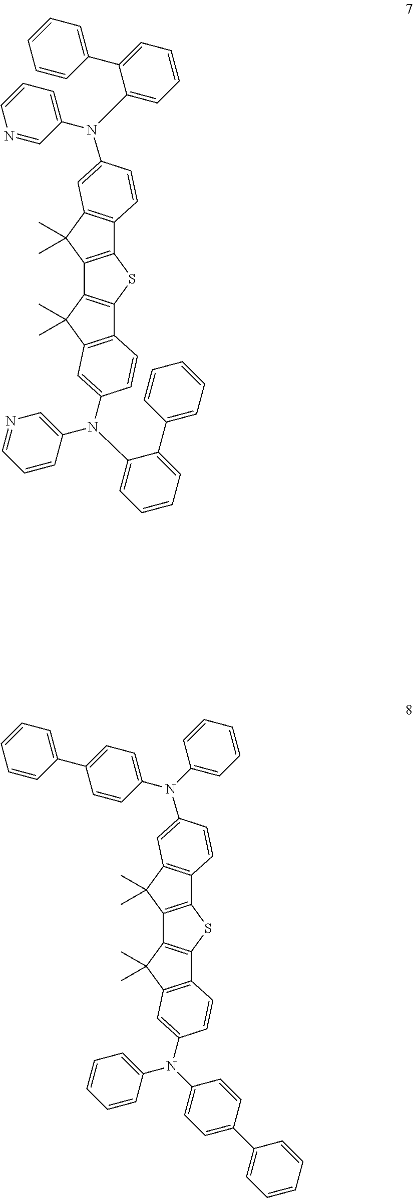 [SMILES: CC1(C)c2cc(N(c3cccnc3)c3ccccc3-c3ccccc3)ccc2-c2sc3c(c21)C(C)(C)c1cc(N(c2cccnc2)c2ccccc2-c2ccccc2)ccc1-3 ; CC1(C)c2cc(N(c3ccccc3)c3ccc(-c4ccccc4)cc3)ccc2-c2sc3c(c21)C(C)(C)c1cc(N(c2ccccc2)c2ccc(-c4ccccc4)cc2)ccc1-3]

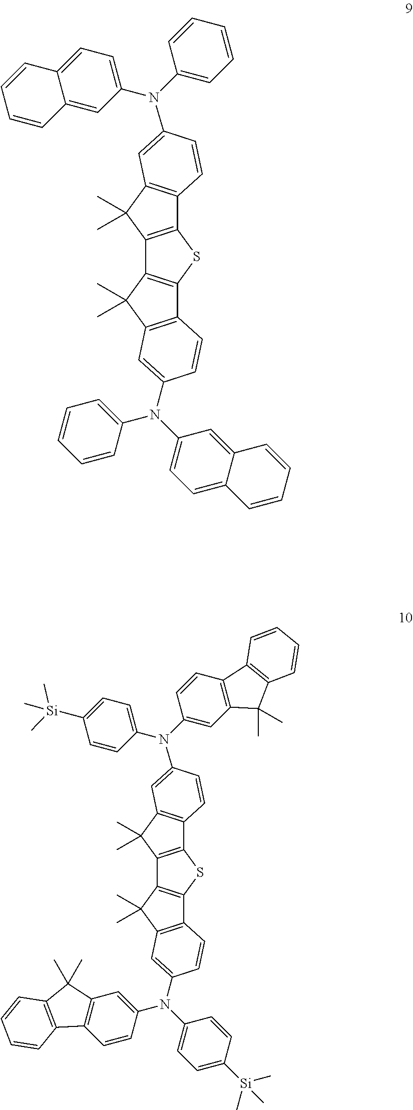 [SMILES: CC1(C)c2cc(N(c3ccccc3)c3ccc4ccccc4c3)ccc2-c2sc3c(c21)C(C)(C)c1cc(N(c2ccccc2)c2ccc4ccccc4c2)ccc1-3 ; CC1(C)c2ccccc2-c2ccc(N(c3ccc([Si](C)(C)C)cc3)c3ccc4c(c3)C(C)(C)c3c-4sc4c3C(C)(C)c3cc(N(c5ccc([Si](C)(C)C)cc5)c5ccc6c(c5)C(C)(C)c5ccccc5-6)ccc3-4)cc21]

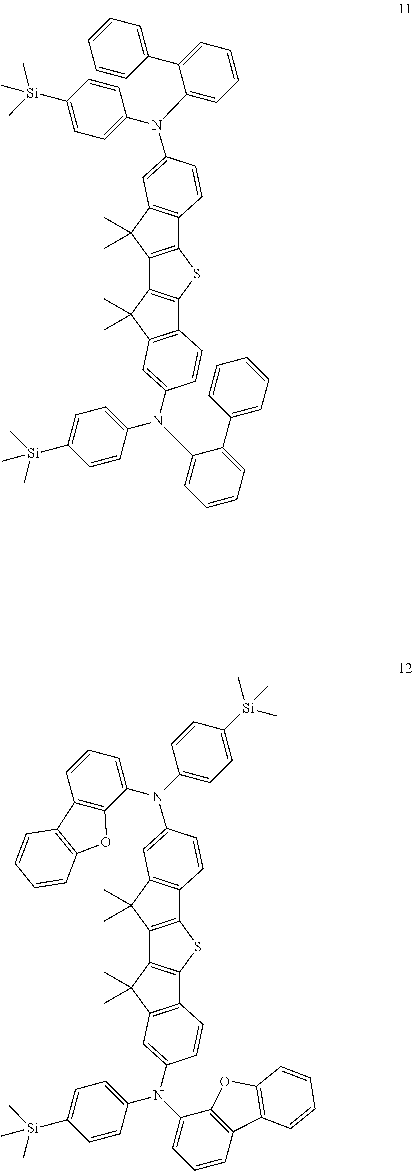 [SMILES: CC1(C)c2cc(N(c3ccc([Si](C)(C)C)cc3)c3ccccc3-c3ccccc3)ccc2-c2sc3c(c21)C(C)(C)c1cc(N(c2ccc([Si](C)(C)C)cc2)c2ccccc2-c2ccccc2)ccc1-3 ; CC1(C)c2cc(N(c3ccc([Si](C)(C)C)cc3)c3cccc4c3oc3ccccc34)ccc2-c2sc3c(c21)C(C)(C)c1cc(N(c2ccc([Si](C)(C)C)cc2)c2cccc4c2oc2ccccc24)ccc1-3]

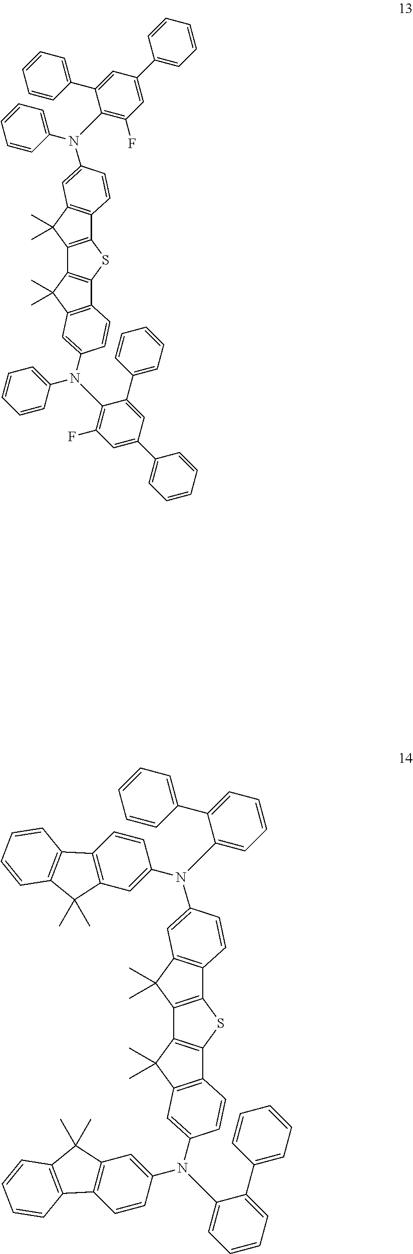 [SMILES: CC1(C)c2cc(N(c3ccccc3)c3c(F)cc(-c4ccccc4)cc3-c3ccccc3)ccc2-c2sc3c(c21)C(C)(C)c1cc(N(c2ccccc2)c2c(F)cc(-c4ccccc4)cc2-c2ccccc2)ccc1-3 ; CC1(C)c2ccccc2-c2ccc(N(c3ccc4c(c3)C(C)(C)c3c-4sc4c3C(C)(C)c3cc(N(c5ccc6c(c5)C(C)(C)c5ccccc5-6)c5ccccc5-c5ccccc5)ccc3-4)c3ccccc3-c3ccccc3)cc21]

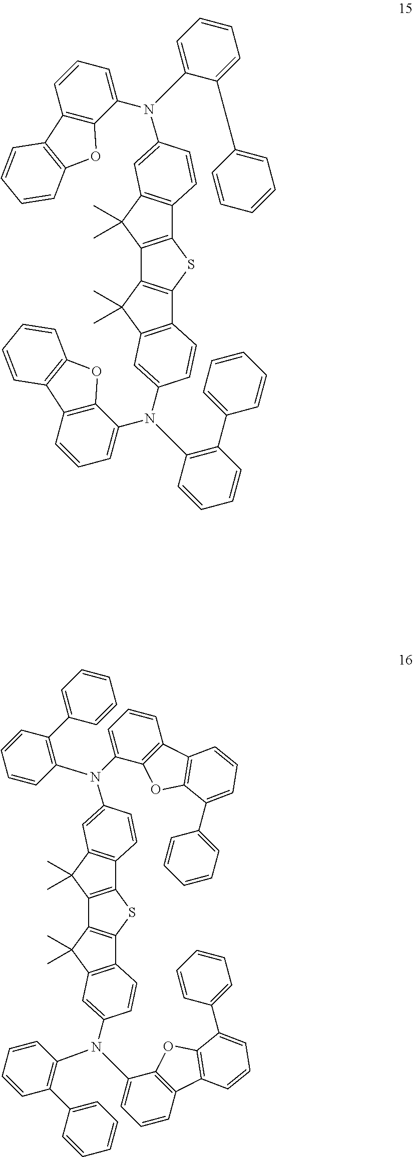 [SMILES: CC1(C)c2cc(N(c3ccccc3-c3ccccc3)c3cccc4c3oc3ccccc34)ccc2-c2sc3c(c21)C(C)(C)c1cc(N(c2ccccc2-c2ccccc2)c2cccc4c2oc2ccccc24)ccc1-3 ; CC1(C)c2cc(N(c3ccccc3-c3ccccc3)c3cccc4c3oc3c(-c5ccccc5)cccc34)ccc2-c2sc3c(c21)C(C)(C)c1cc(N(c2ccccc2-c2ccccc2)c2cccc4c2oc2c(-c5ccccc5)cccc24)ccc1-3]

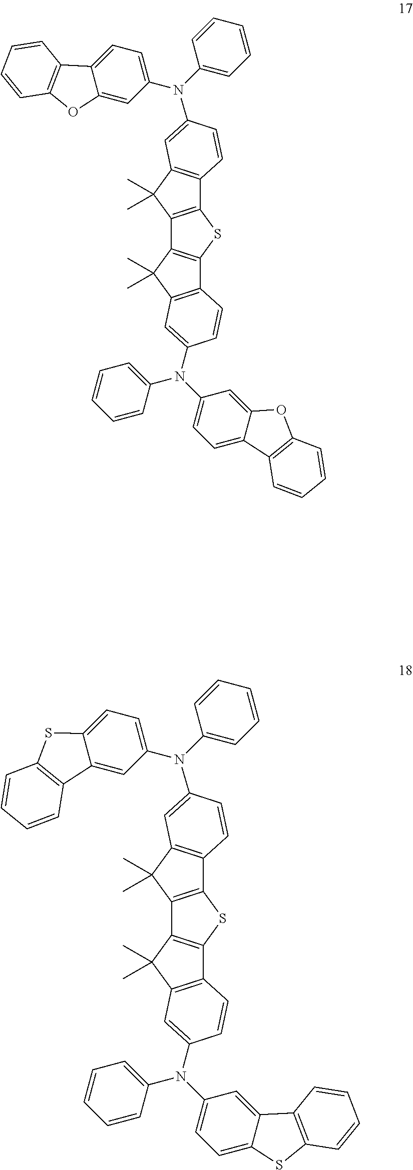 [SMILES: CC1(C)c2cc(N(c3ccccc3)c3ccc4c(c3)oc3ccccc34)ccc2-c2sc3c(c21)C(C)(C)c1cc(N(c2ccccc2)c2ccc4c(c2)oc2ccccc24)ccc1-3 ; CC1(C)c2cc(N(c3ccccc3)c3ccc4sc5ccccc5c4c3)ccc2-c2sc3c(c21)C(C)(C)c1cc(N(c2ccccc2)c2ccc4sc5ccccc5c4c2)ccc1-3]

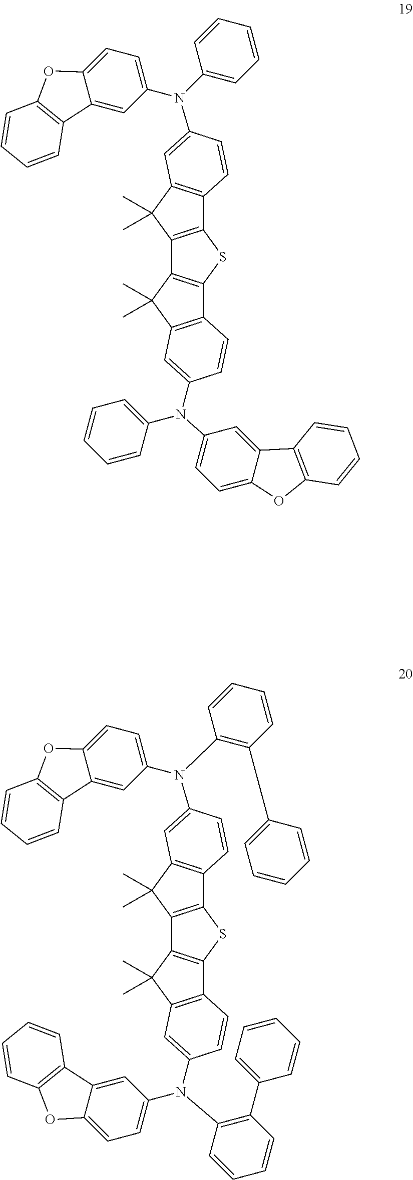 [SMILES: CC1(C)c2cc(N(c3ccccc3)c3ccc4oc5ccccc5c4c3)ccc2-c2sc3c(c21)C(C)(C)c1cc(N(c2ccccc2)c2ccc4oc5ccccc5c4c2)ccc1-3 ; CC1(C)c2cc(N(c3ccc4oc5ccccc5c4c3)c3ccccc3-c3ccccc3)ccc2-c2sc3c(c21)C(C)(C)c1cc(N(c2ccc4oc5ccccc5c4c2)c2ccccc2-c2ccccc2)ccc1-3]

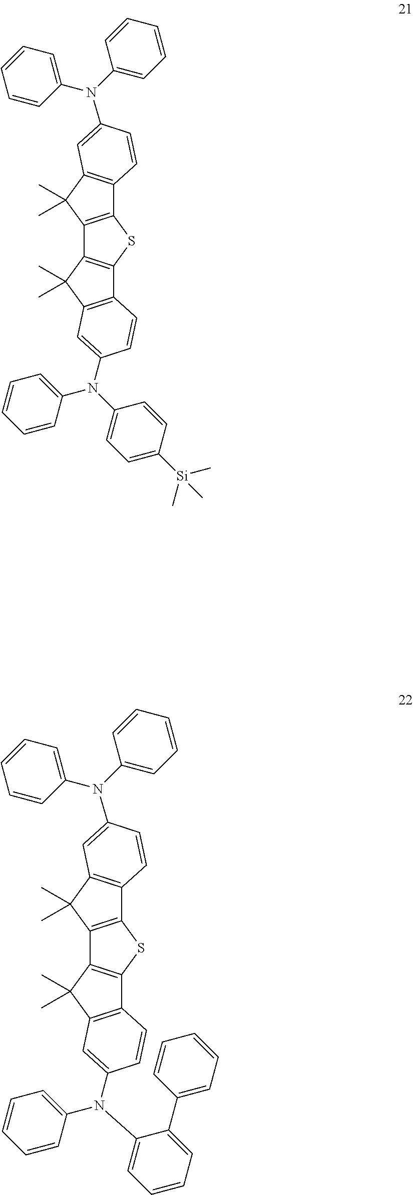 [SMILES: CC1(C)c2cc(N(c3ccccc3)c3ccccc3)ccc2-c2sc3c(c21)C(C)(C)c1cc(N(c2ccccc2)c2ccc([Si](C)(C)C)cc2)ccc1-3 ; CC1(C)c2cc(N(c3ccccc3)c3ccccc3)ccc2-c2sc3c(c21)C(C)(C)c1cc(N(c2ccccc2)c2ccccc2-c2ccccc2)ccc1-3]

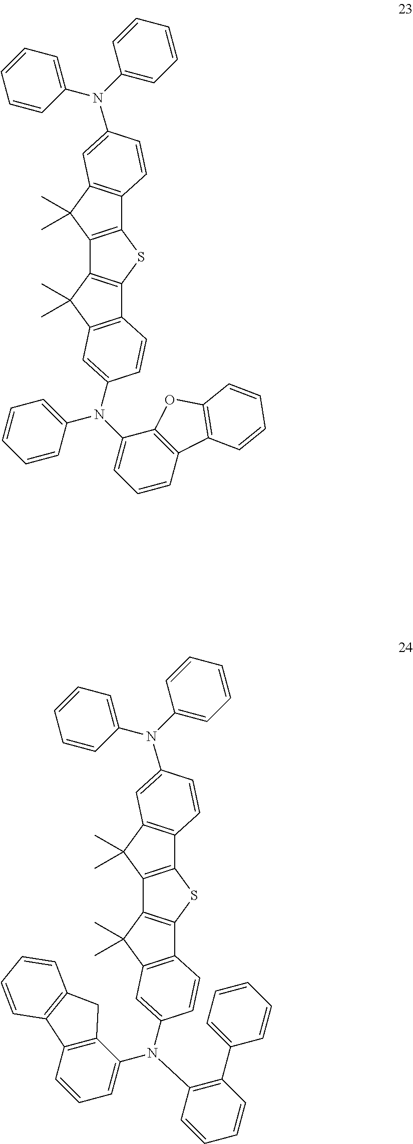 [SMILES: CC1(C)c2cc(N(c3ccccc3)c3ccccc3)ccc2-c2sc3c(c21)C(C)(C)c1cc(N(c2ccccc2)c2cccc4c2oc2ccccc24)ccc1-3 ; CC1(C)c2cc(N(c3ccccc3)c3ccccc3)ccc2-c2sc3c(c21)C(C)(C)c1cc(N(c2ccccc2-c2ccccc2)c2cccc4c2Cc2ccccc2-4)ccc1-3]

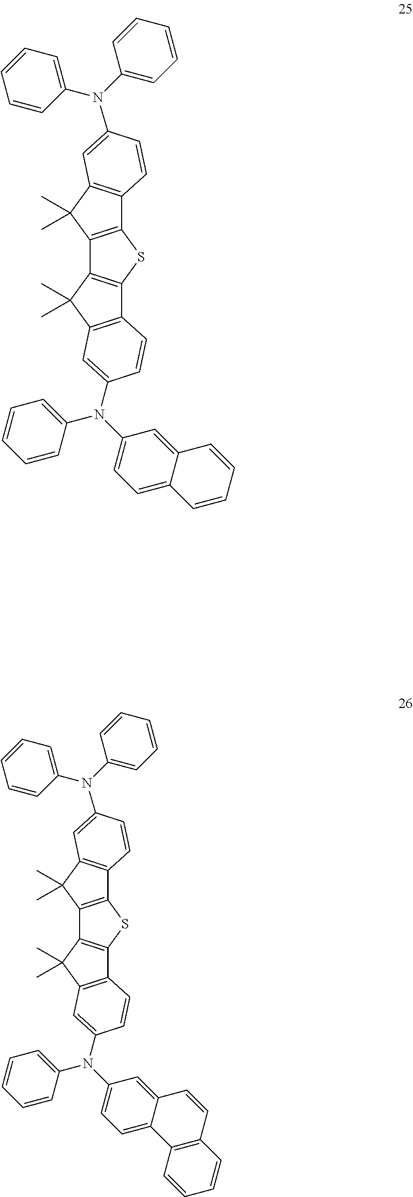 [SMILES: CC1(C)c2cc(N(c3ccccc3)c3ccccc3)ccc2-c2sc3c(c21)C(C)(C)c1cc(N(c2ccccc2)c2ccc4ccccc4c2)ccc1-3 ; CC1(C)c2cc(N(c3ccccc3)c3ccccc3)ccc2-c2sc3c(c21)C(C)(C)c1cc(N(c2ccccc2)c2ccc4c(ccc5ccccc54)c2)ccc1-3]

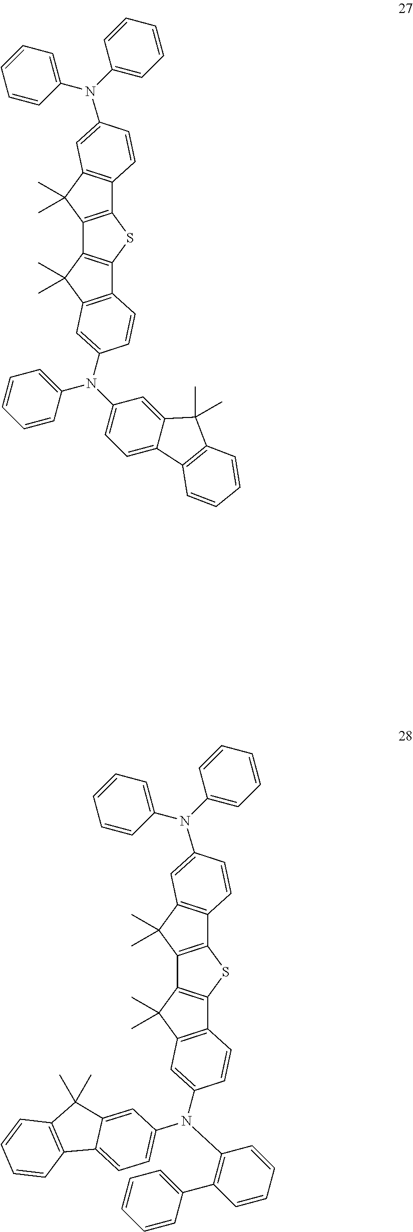 [SMILES: CC1(C)c2ccccc2-c2ccc(N(c3ccccc3)c3ccc4c(c3)C(C)(C)c3c-4sc4c3C(C)(C)c3cc(N(c5ccccc5)c5ccccc5)ccc3-4)cc21 ; CC1(C)c2ccccc2-c2ccc(N(c3ccc4c(c3)C(C)(C)c3c-4sc4c3C(C)(C)c3cc(N(c5ccccc5)c5ccccc5)ccc3-4)c3ccccc3-c3ccccc3)cc21]

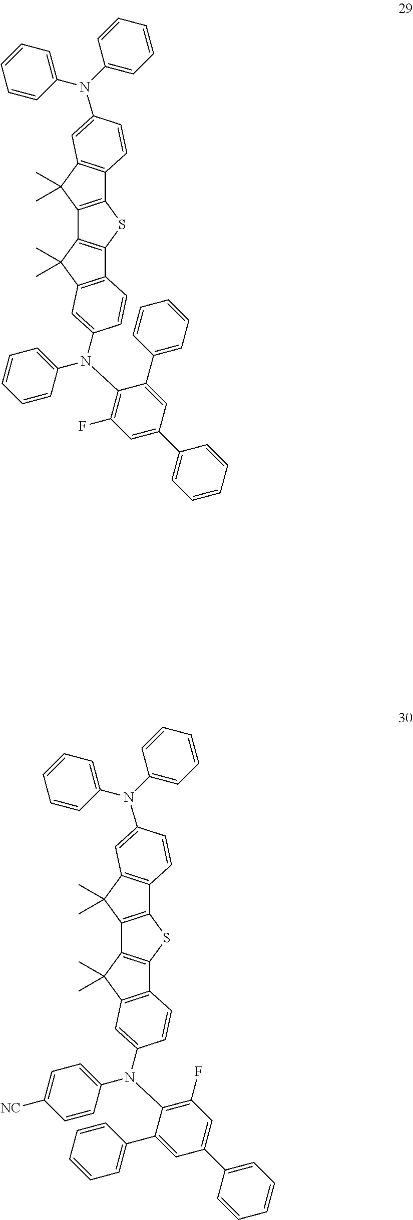 [SMILES: CC1(C)c2cc(N(c3ccccc3)c3ccccc3)ccc2-c2sc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccccc3)ccc2-c2sc3c(c21)C(C)(C)c1cc(N(c2ccc(C#N)cc2)c2c(F)cc(-c4ccccc4)cc2-c2ccccc2)ccc1-3]

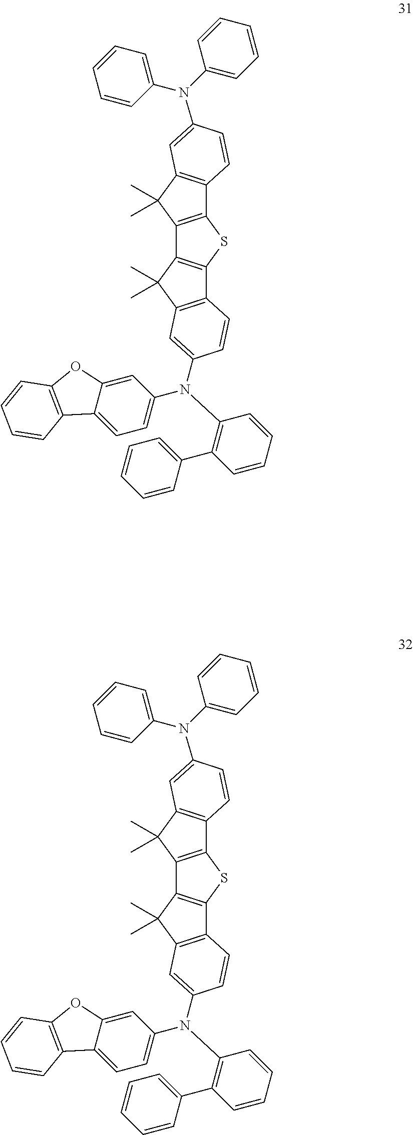 [SMILES: CC1(C)c2cc(N(c3ccccc3)c3ccccc3)ccc2-c2sc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccccc3)ccc2-c2sc3c(c21)C(C)(C)c1cc(N(c2ccc4c(c2)oc2ccccc24)c2ccccc2-c2ccccc2)ccc1-3]

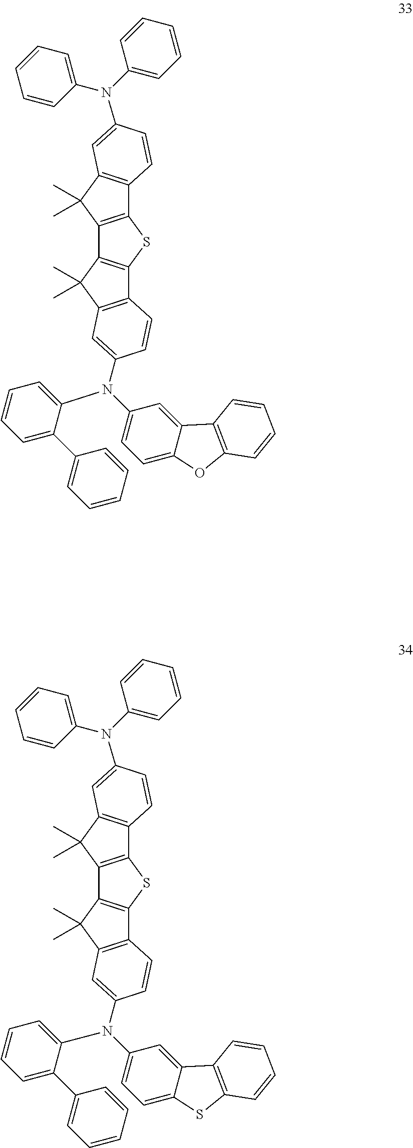 [SMILES: CC1(C)c2cc(N(c3ccccc3)c3ccccc3)ccc2-c2sc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2sc3c(c21)C(C)(C)c1cc(N(c2ccc4sc5ccccc5c4c2)c2ccccc2-c2ccccc2)ccc1-3]

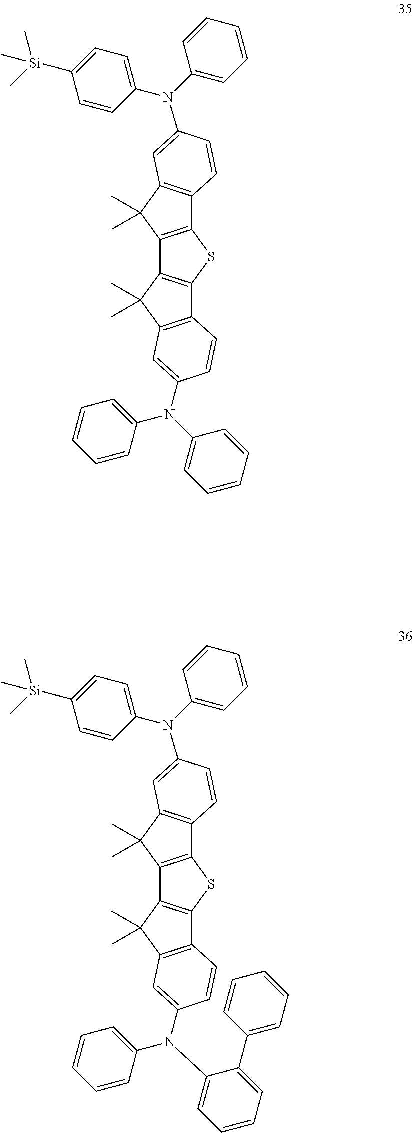 [SMILES: CC1(C)c2cc(N(c3ccccc3)c3ccccc3)ccc2-c2sc3c(c21)C(C)(C)c1cc(N(c2ccccc2)c2ccc([Si](C)(C)C)cc2)ccc1-3 ; CC1(C)c2cc(N(c3ccccc3)c3ccc([Si](C)(C)C)cc3)ccc2-c2sc3c(c21)C(C)(C)c1cc(N(c2ccccc2)c2ccccc2-c2ccccc2)ccc1-3]

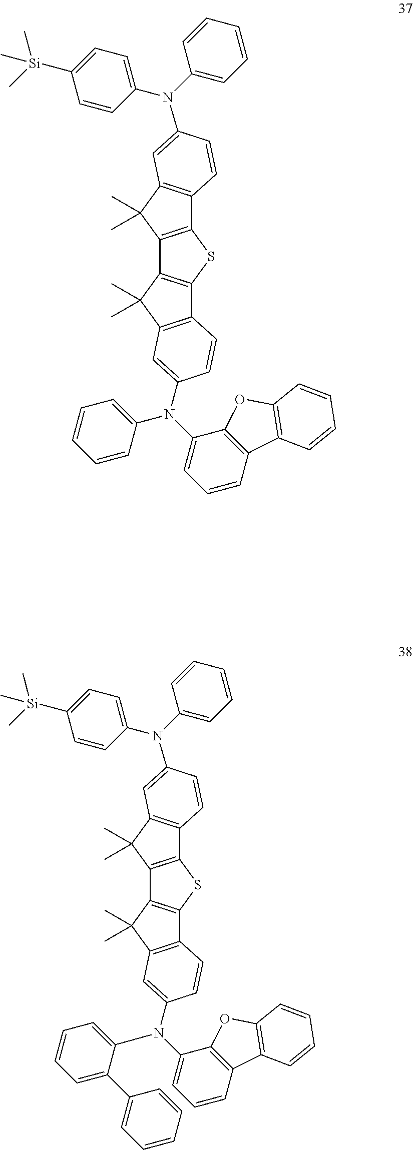 [SMILES: CC1(C)c2cc(N(c3ccccc3)c3ccc([Si](C)(C)C)cc3)ccc2-c2sc3c(c21)C(C)(C)c1cc(N(c2ccccc2)c2cccc4c2oc2ccccc24)ccc1-3 ; CC1(C)c2cc(N(c3ccccc3)c3ccc([Si](C)(C)C)cc3)ccc2-c2sc3c(c21)C(C)(C)c1cc(N(c2ccccc2-c2ccccc2)c2cccc4c2oc2ccccc24)ccc1-3]

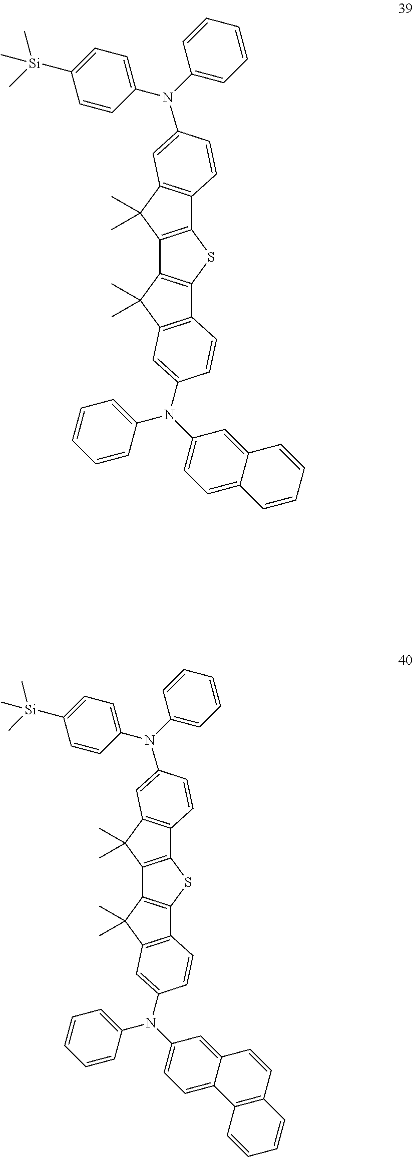 [SMILES: CC1(C)c2cc(N(c3ccccc3)c3ccc([Si](C)(C)C)cc3)ccc2-c2sc3c(c21)C(C)(C)c1cc(N(c2ccccc2)c2ccc4ccccc4c2)ccc1-3 ; CC1(C)c2cc(N(c3ccccc3)c3ccc([Si](C)(C)C)cc3)ccc2-c2sc3c(c21)C(C)(C)c1cc(N(c2ccccc2)c2ccc4c(ccc5ccccc54)c2)ccc1-3]

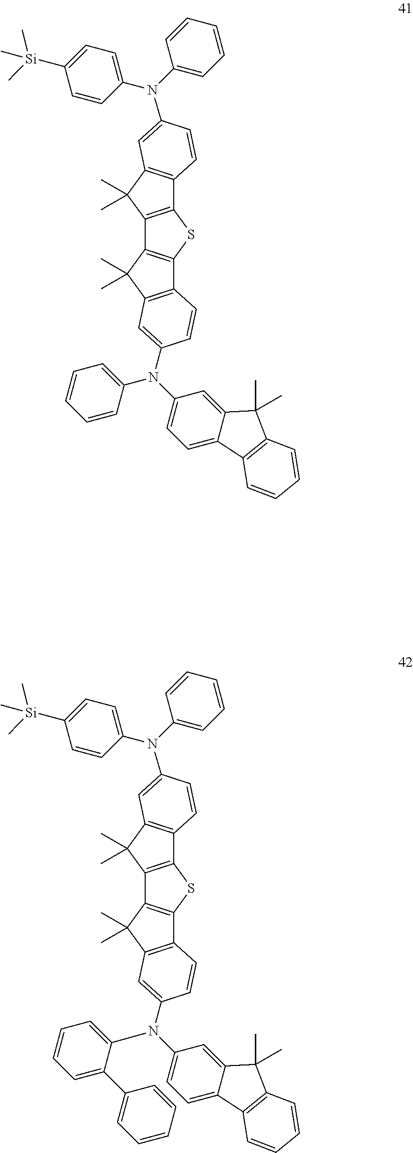 [SMILES: CC1(C)c2ccccc2-c2ccc(N(c3ccccc3)c3ccc4c(c3)C(C)(C)c3c-4sc4c3C(C)(C)c3cc(N(c5ccccc5)c5ccc([Si](C)(C)C)cc5)ccc3-4)cc21 ; CC1(C)c2ccccc2-c2ccc(N(c3ccc4c(c3)C(C)(C)c3c-4sc4c3C(C)(C)c3cc(N(c5ccccc5)c5ccc([Si](C)(C)C)cc5)ccc3-4)c3ccccc3-c3ccccc3)cc21]

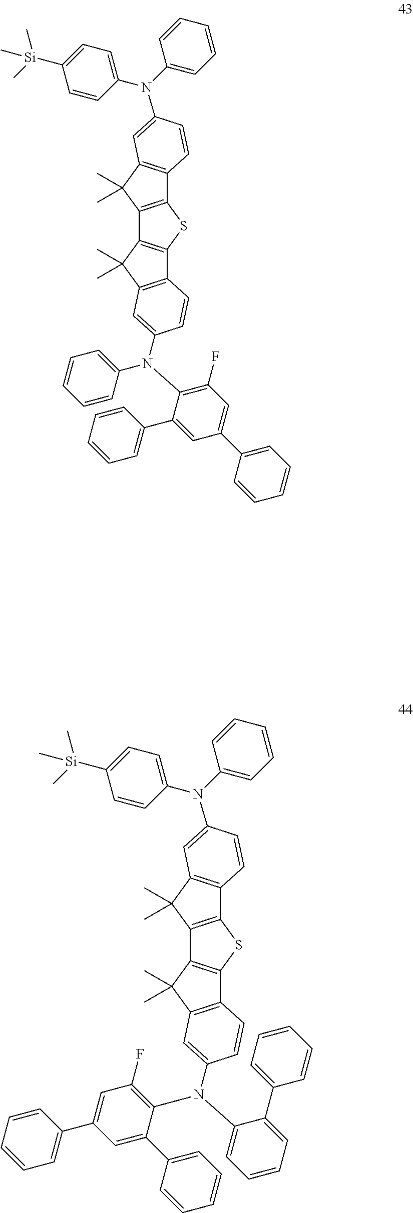 [SMILES: CC1(C)c2cc(N(c3ccccc3)c3ccc([Si](C)(C)C)cc3)ccc2-c2sc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccc([Si](C)(C)C)cc3)ccc2-c2sc3c(c21)C(C)(C)c1cc(N(c2ccccc2-c2ccccc2)c2c(F)cc(-c4ccccc4)cc2-c2ccccc2)ccc1-3]

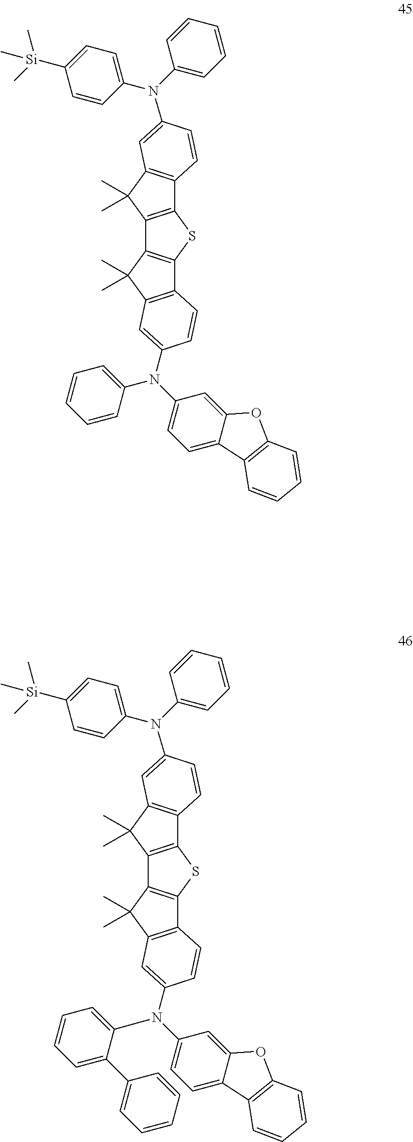 [SMILES: CC1(C)c2cc(N(c3ccccc3)c3ccc([Si](C)(C)C)cc3)ccc2-c2sc3c(c21)C(C)(C)c1cc(N(c2ccccc2)c2ccc4c(c2)oc2ccccc24)ccc1-3 ; CC1(C)c2cc(N(c3ccccc3)c3ccc([Si](C)(C)C)cc3)ccc2-c2sc3c(c21)C(C)(C)c1cc(N(c2ccc4c(c2)oc2ccccc24)c2ccccc2-c2ccccc2)ccc1-3]

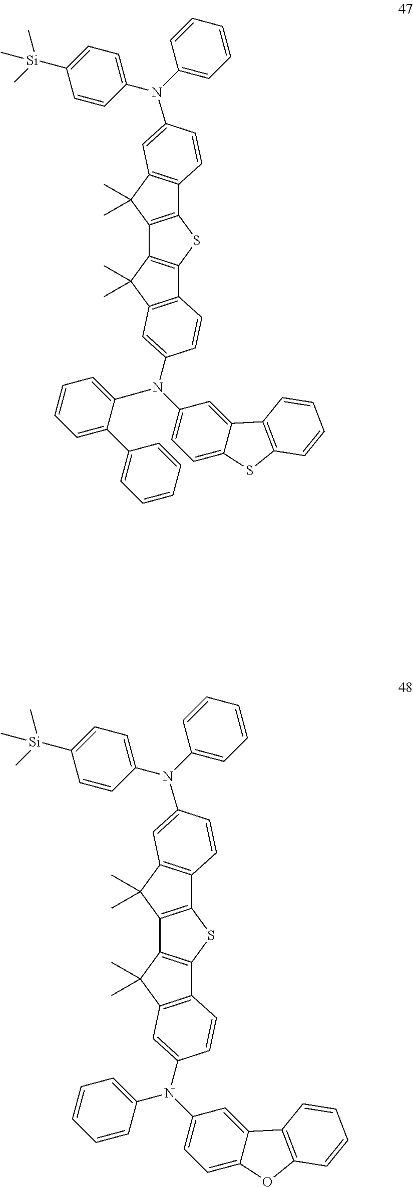 [SMILES: CC1(C)c2cc(N(c3ccccc3)c3ccc([Si](C)(C)C)cc3)ccc2-c2sc3c(c21)C(C)(C)c1cc(N(c2ccc4sc5ccccc5c4c2)c2ccccc2-c2ccccc2)ccc1-3 ; CC1(C)c2cc(N(c3ccccc3)c3ccc([Si](C)(C)C)cc3)ccc2-c2sc3c(c21)C(C)(C)c1cc(N(c2ccccc2)c2ccc4oc5ccccc5c4c2)ccc1-3]

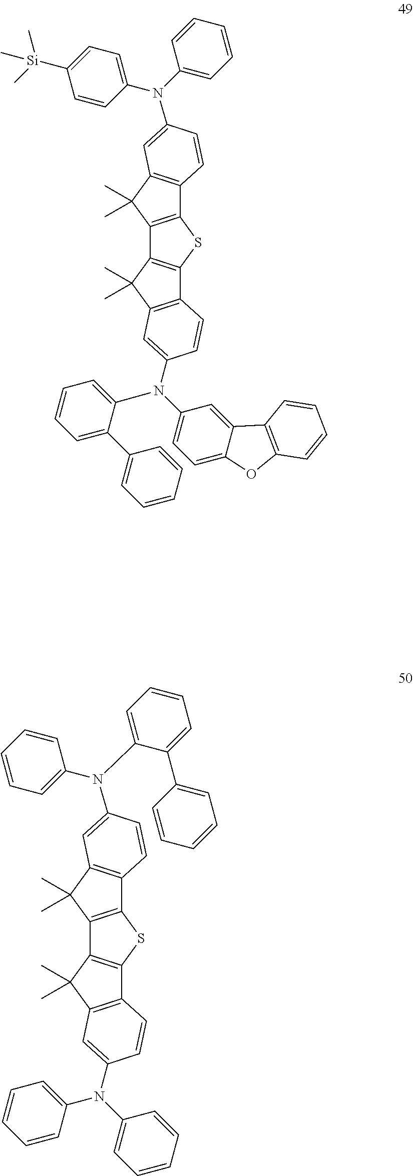 [SMILES: CC1(C)c2cc(N(c3ccccc3)c3ccc([Si](C)(C)C)cc3)ccc2-c2sc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2sc3c(c21)C(C)(C)c1cc(N(c2ccccc2)c2ccccc2-c2ccccc2)ccc1-3]

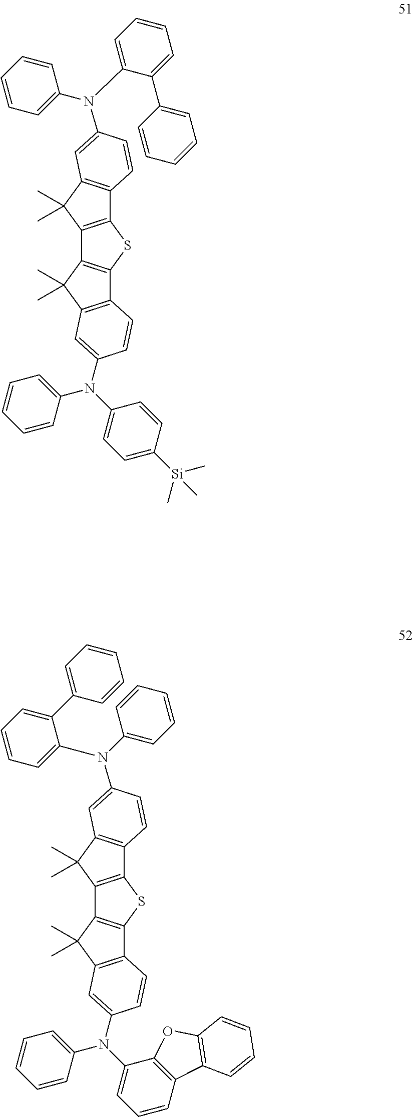 [SMILES: CC1(C)c2cc(N(c3ccccc3)c3ccc([Si](C)(C)C)cc3)ccc2-c2sc3c(c21)C(C)(C)c1cc(N(c2ccccc2)c2ccccc2-c2ccccc2)ccc1-3 ; CC1(C)c2cc(N(c3ccccc3)c3ccccc3-c3ccccc3)ccc2-c2sc3c(c21)C(C)(C)c1cc(N(c2ccccc2)c2cccc4c2oc2ccccc24)ccc1-3]

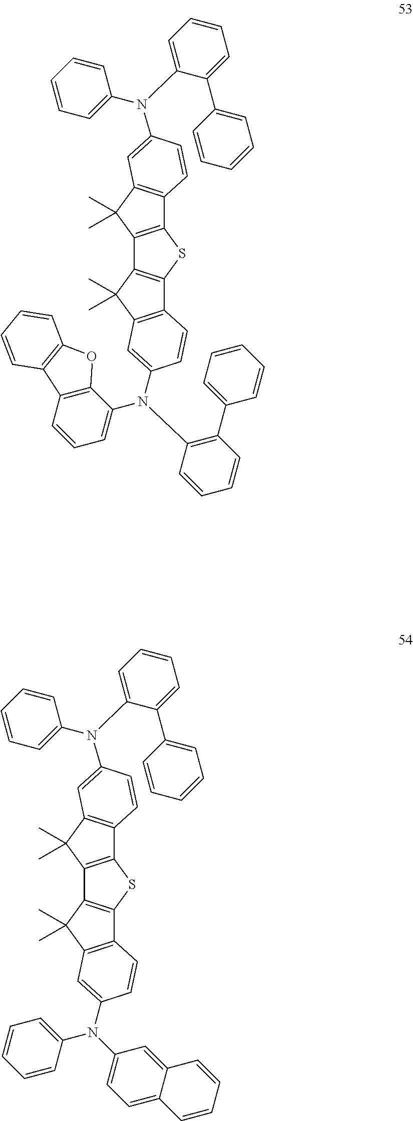 [SMILES: CC1(C)c2cc(N(c3ccccc3)c3ccccc3-c3ccccc3)ccc2-c2sc3c(c21)C(C)(C)c1cc(N(c2ccccc2-c2ccccc2)c2cccc4c2oc2ccccc24)ccc1-3 ; CC1(C)c2cc(N(c3ccccc3)c3ccc4ccccc4c3)ccc2-c2sc3c(c21)C(C)(C)c1cc(N(c2ccccc2)c2ccccc2-c2ccccc2)ccc1-3]

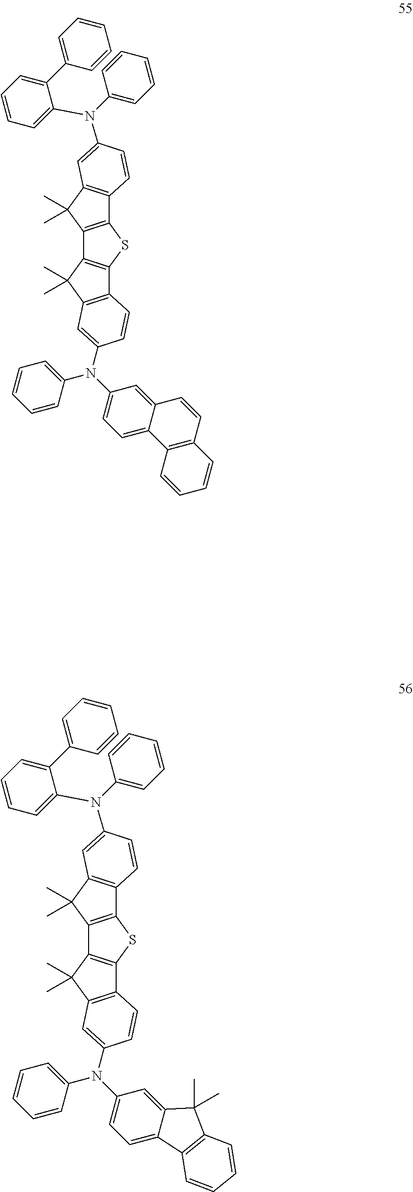 [SMILES: CC1(C)c2cc(N(c3ccccc3)c3ccc4c(ccc5ccccc54)c3)ccc2-c2sc3c(c21)C(C)(C)c1cc(N(c2ccccc2)c2ccccc2-c2ccccc2)ccc1-3 ; CC1(C)c2ccccc2-c2ccc(N(c3ccccc3)c3ccc4c(c3)C(C)(C)c3c-4sc4c3C(C)(C)c3cc(N(c5ccccc5)c5ccccc5-c5ccccc5)ccc3-4)cc21]

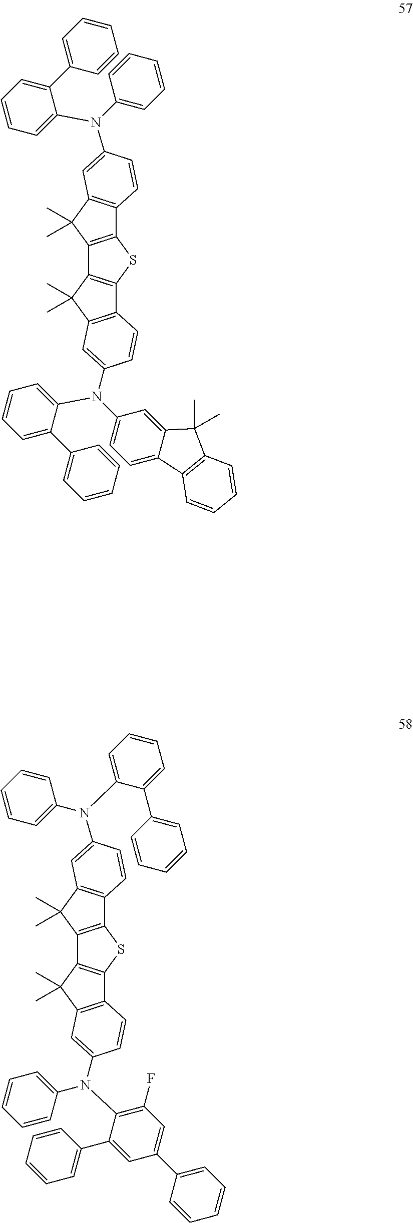 [SMILES: CC1(C)c2ccccc2-c2ccc(N(c3ccc4c(c3)C(C)(C)c3c-4sc4c3C(C)(C)c3cc(N(c5ccccc5)c5ccccc5-c5ccccc5)ccc3-4)c3ccccc3-c3ccccc3)cc21 ; CC1(C)c2cc(N(c3ccccc3)c3ccccc3-c3ccccc3)ccc2-c2sc3c(c21)C(C)(C)c1cc(N(c2ccccc2)c2c(F)cc(-c4ccccc4)cc2-c2ccccc2)ccc1-3]

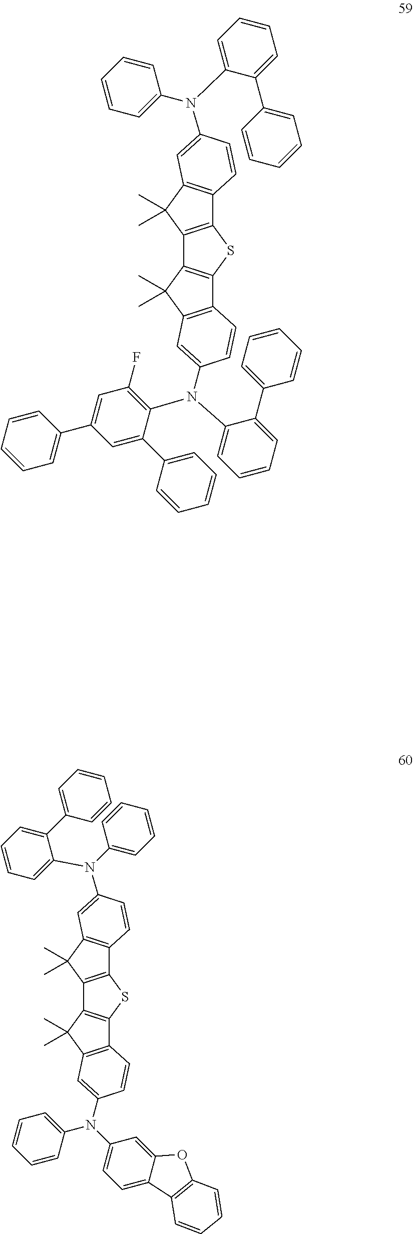 [SMILES: CC1(C)c2cc(N(c3ccccc3)c3ccccc3-c3ccccc3)ccc2-c2sc3c(c21)C(C)(C)c1cc(N(c2ccccc2-c2ccccc2)c2c(F)cc(-c4ccccc4)cc2-c2ccccc2)ccc1-3 ; CC1(C)c2cc(N(c3ccccc3)c3ccc4c(c3)oc3ccccc34)ccc2-c2sc3c(c21)C(C)(C)c1cc(N(c2ccccc2)c2ccccc2-c2ccccc2)ccc1-3]

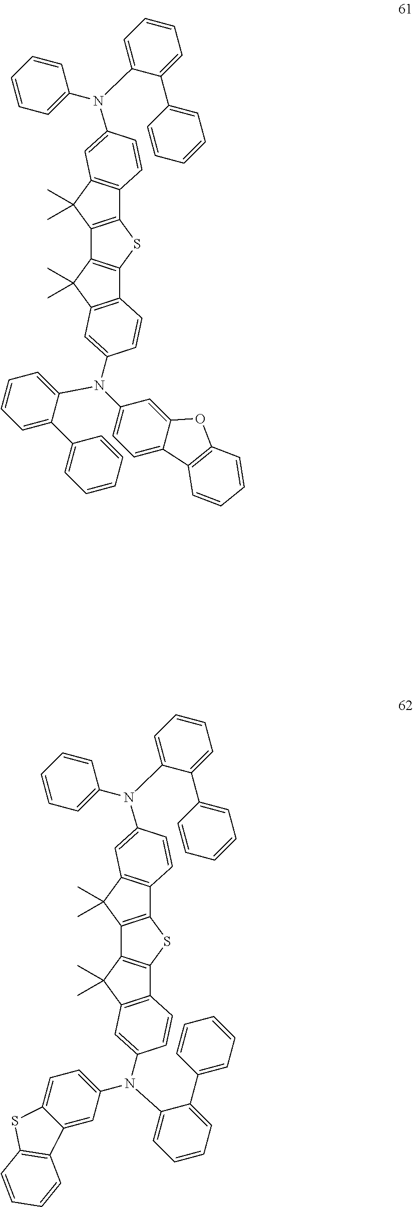 [SMILES: CC1(C)c2cc(N(c3ccccc3)c3ccccc3-c3ccccc3)ccc2-c2sc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccccc3-c3ccccc3)ccc2-c2sc3c(c21)C(C)(C)c1cc(N(c2ccc4sc5ccccc5c4c2)c2ccccc2-c2ccccc2)ccc1-3]

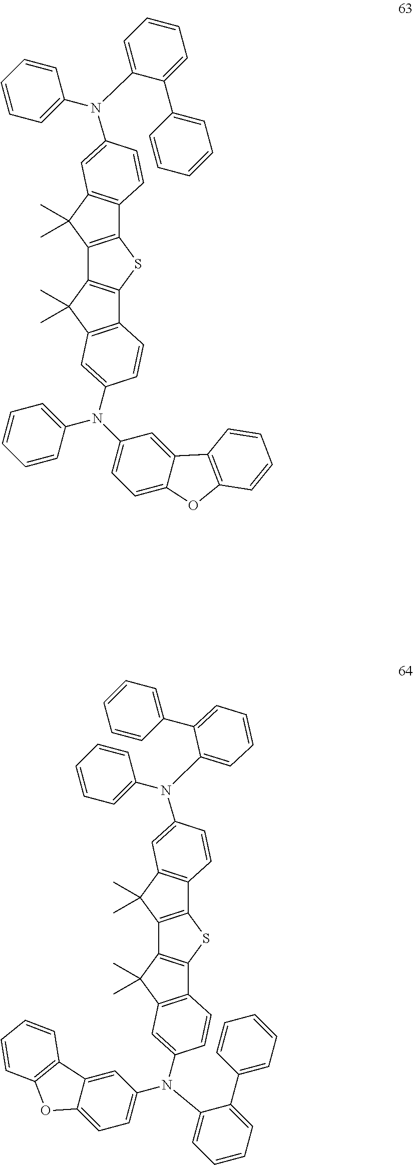 [SMILES: CC1(C)c2cc(N(c3ccccc3)c3ccc4oc5ccccc5c4c3)ccc2-c2sc3c(c21)C(C)(C)c1cc(N(c2ccccc2)c2ccccc2-c2ccccc2)ccc1-3 ; CC1(C)c2cc(N(c3ccccc3)c3ccccc3-c3ccccc3)ccc2-c2sc3c(c21)C(C)(C)c1cc(N(c2ccc4oc5ccccc5c4c2)c2ccccc2-c2ccccc2)ccc1-3]

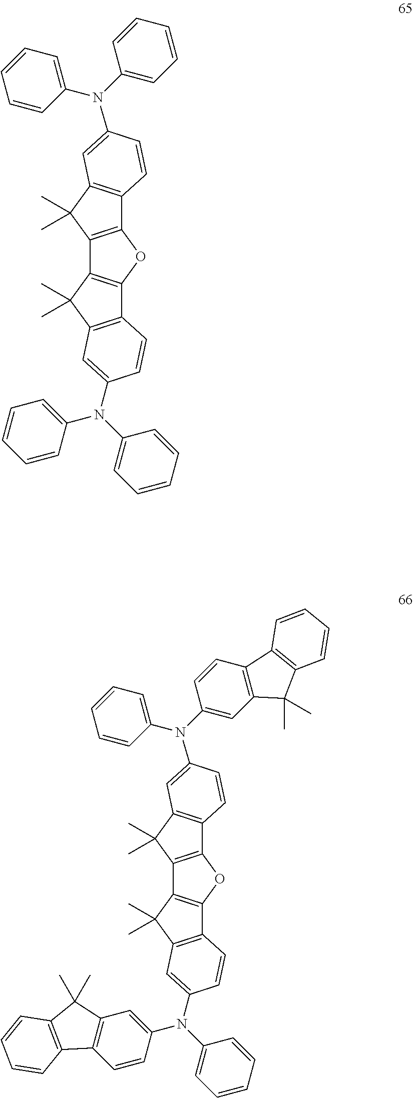 [SMILES: CC1(C)c2cc(N(c3ccccc3)c3ccccc3)ccc2-c2oc3c(c21)C(C)(C)c1cc(N(c2ccccc2)c2ccccc2)ccc1-3 ; CC1(C)c2ccccc2-c2ccc(N(c3ccccc3)c3ccc4c(c3)C(C)(C)c3c-4oc4c3C(C)(C)c3cc(N(c5ccccc5)c5ccc6c(c5)C(C)(C)c5ccccc5-6)ccc3-4)cc21]

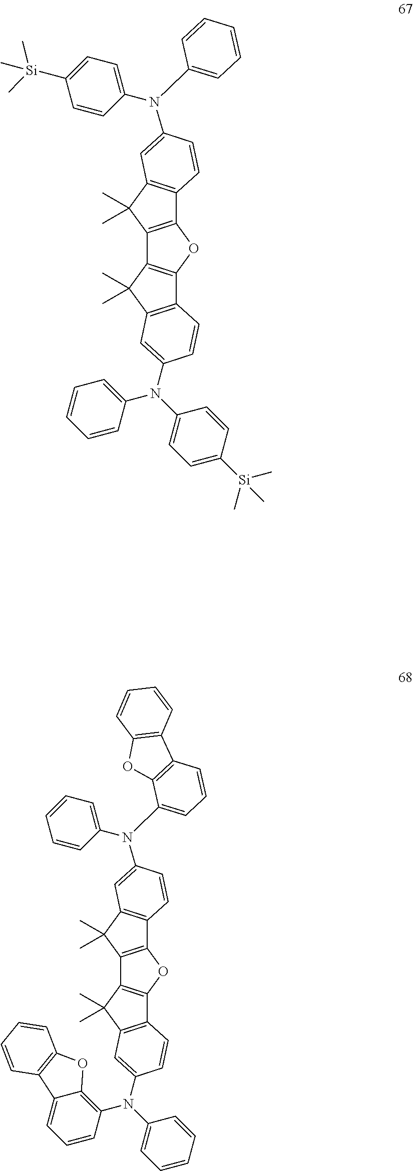

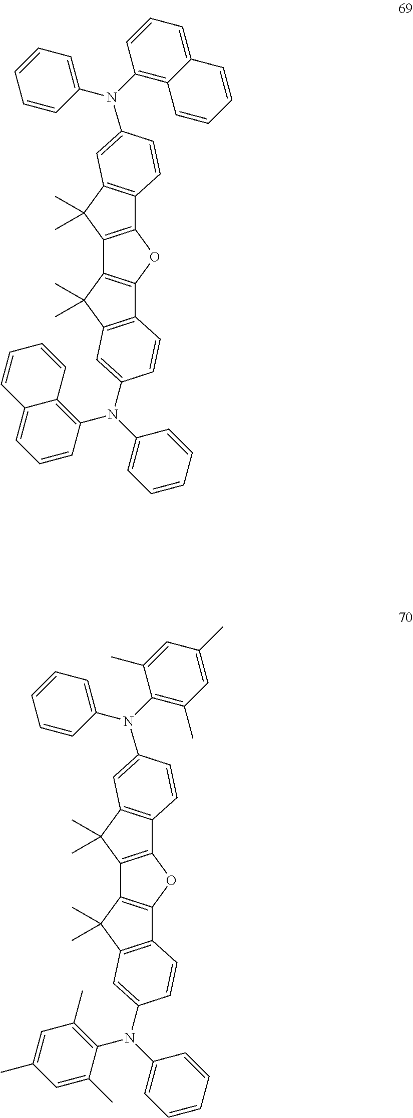

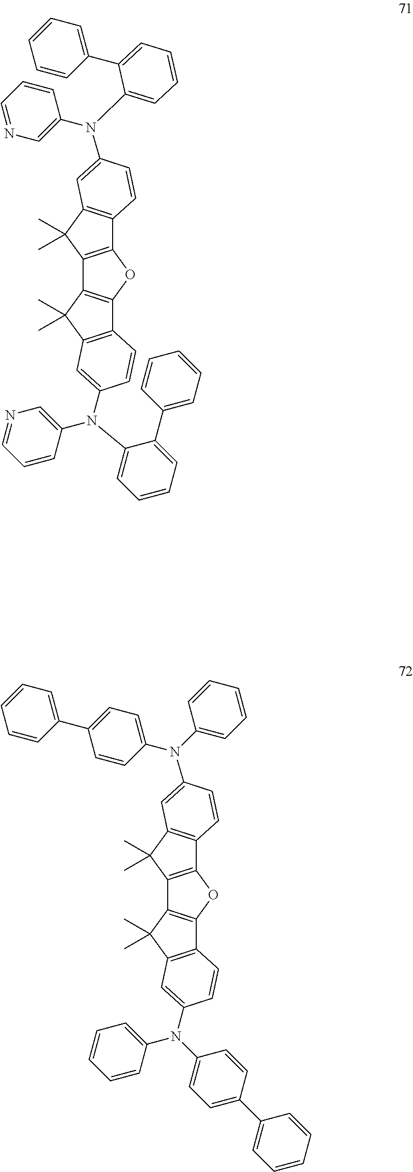 [SMILES: CC1(C)c2cc(N(c3cccnc3)c3ccccc3-c3ccccc3)ccc2-c2oc3c(c21)C(C)(C)c1cc(N(c2cccnc2)c2ccccc2-c2ccccc2)ccc1-3 ; CC1(C)c2cc(N(c3ccccc3)c3ccc(-c4ccccc4)cc3)ccc2-c2oc3c(c21)C(C)(C)c1cc(N(c2ccccc2)c2ccc(-c4ccccc4)cc2)ccc1-3]

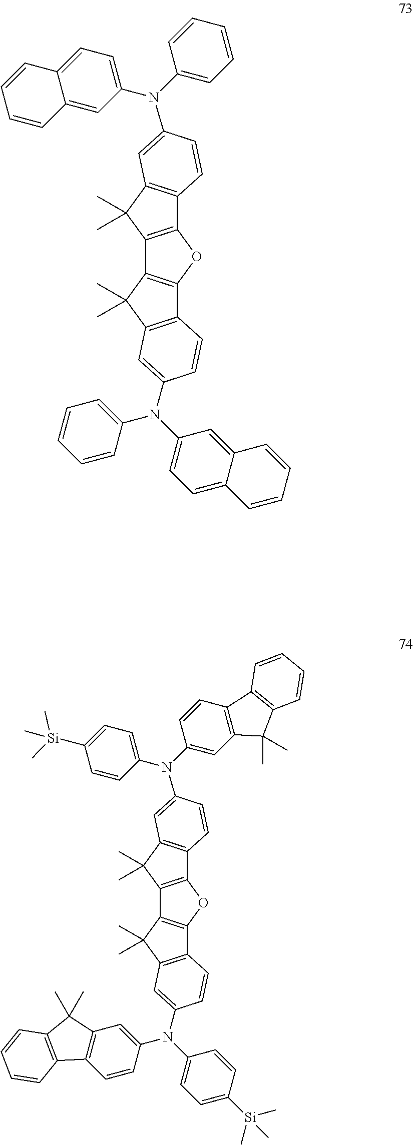 [SMILES: CC1(C)c2cc(N(c3ccccc3)c3ccc4ccccc4c3)ccc2-c2oc3c(c21)C(C)(C)c1cc(N(c2ccccc2)c2ccc4ccccc4c2)ccc1-3 ; CC1(C)c2ccccc2-c2ccc(N(c3ccc([Si](C)(C)C)cc3)c3ccc4c(c3)C(C)(C)c3c-4oc4c3C(C)(C)c3cc(N(c5ccc([Si](C)(C)C)cc5)c5ccc6c(c5)C(C)(C)c5ccccc5-6)ccc3-4)cc21]

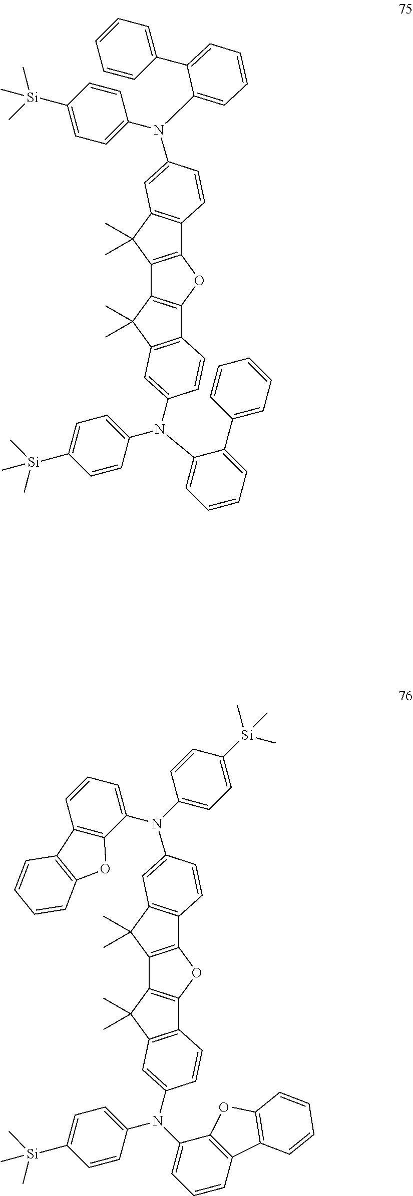 [SMILES: CC1(C)c2cc(N(c3ccc([Si](C)(C)C)cc3)c3ccccc3-c3ccccc3)ccc2-c2oc3c(c21)C(C)(C)c1cc(N(c2ccc([Si](C)(C)C)cc2)c2ccccc2-c2ccccc2)ccc1-3 ; CC1(C)c2cc(N(c3ccc([Si](C)(C)C)cc3)c3cccc4c3oc3ccccc34)ccc2-c2oc3c(c21)C(C)(C)c1cc(N(c2ccc([Si](C)(C)C)cc2)c2cccc4c2oc2ccccc24)ccc1-3]

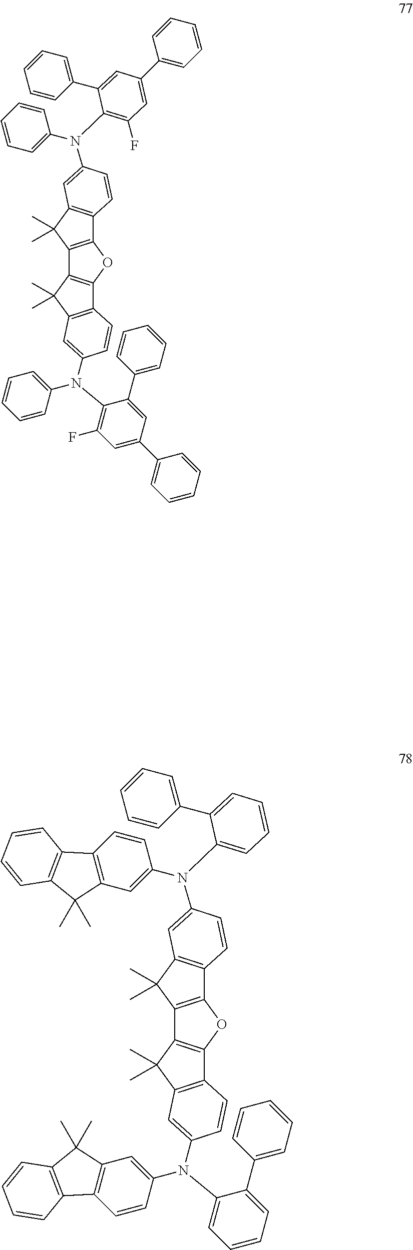 [SMILES: CC1(C)c2cc(N(c3ccccc3)c3c(F)cc(-c4ccccc4)cc3-c3ccccc3)ccc2-c2oc3c(c21)C(C)(C)c1cc(N(c2ccccc2)c2c(F)cc(-c4ccccc4)cc2-c2ccccc2)ccc1-3 ; CC1(C)c2ccccc2-c2ccc(N(c3ccc4c(c3)C(C)(C)c3c-4oc4c3C(C)(C)c3cc(N(c5ccc6c(c5)C(C)(C)c5ccccc5-6)c5ccccc5-c5ccccc5)ccc3-4)c3ccccc3-c3ccccc3)cc21]

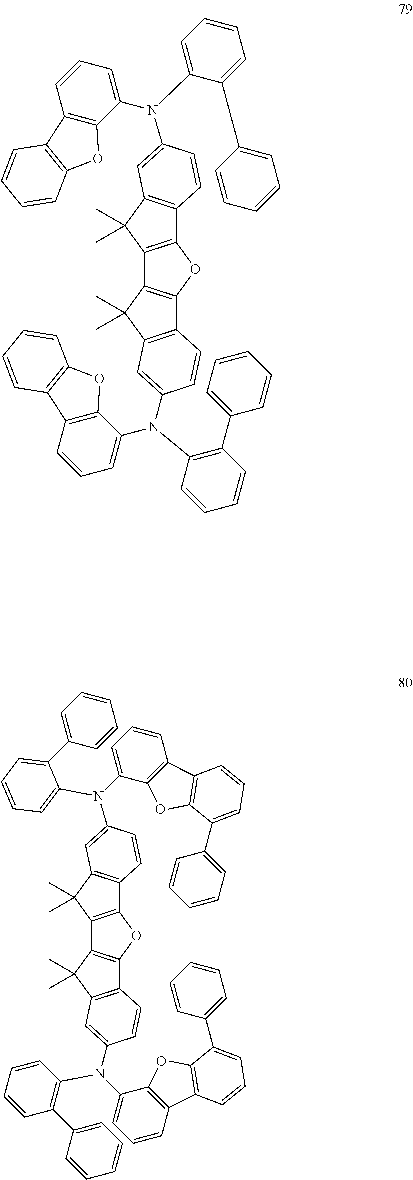 [SMILES: CC1(C)c2cc(N(c3ccccc3-c3ccccc3)c3cccc4c3oc3ccccc34)ccc2-c2oc3c(c21)C(C)(C)c1cc(N(c2ccccc2-c2ccccc2)c2cccc4c2oc2ccccc24)ccc1-3 ; CC1(C)c2cc(N(c3ccccc3-c3ccccc3)c3cccc4c3oc3c(-c5ccccc5)cccc34)ccc2-c2oc3c(c21)C(C)(C)c1cc(N(c2ccccc2-c2ccccc2)c2cccc4c2oc2c(-c5ccccc5)cccc24)ccc1-3]

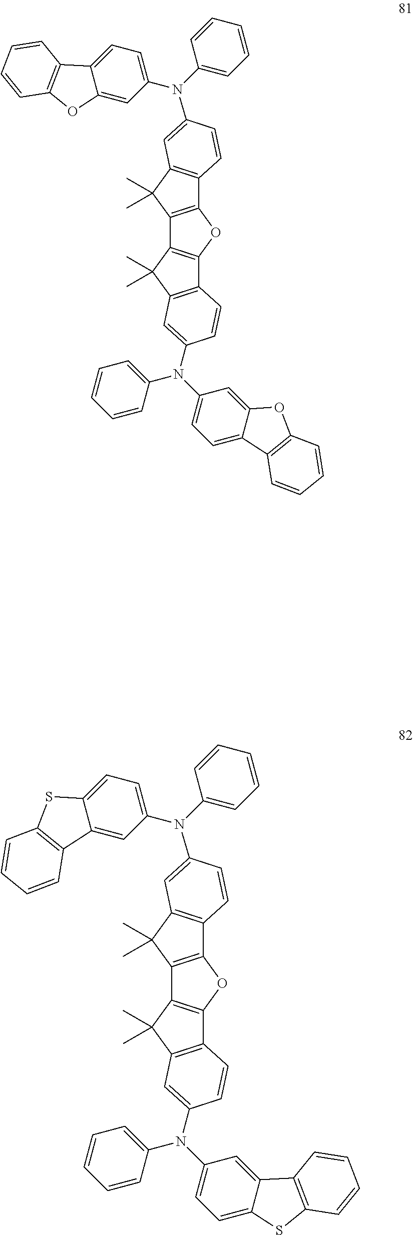 [SMILES: CC1(C)c2cc(N(c3ccccc3)c3ccc4c(c3)oc3ccccc34)ccc2-c2oc3c(c21)C(C)(C)c1cc(N(c2ccccc2)c2ccc4c(c2)oc2ccccc24)ccc1-3 ; CC1(C)c2cc(N(c3ccccc3)c3ccc4sc5ccccc5c4c3)ccc2-c2oc3c(c21)C(C)(C)c1cc(N(c2ccccc2)c2ccc4sc5ccccc5c4c2)ccc1-3]

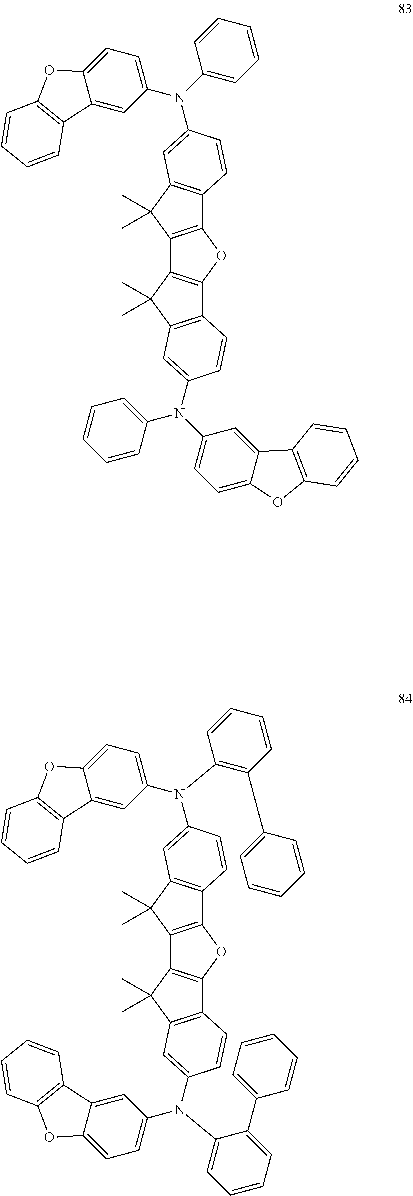 [SMILES: CC1(C)c2cc(N(c3ccccc3)c3ccc4oc5ccccc5c4c3)ccc2-c2oc3c(c21)C(C)(C)c1cc(N(c2ccccc2)c2ccc4oc5ccccc5c4c2)ccc1-3 ; CC1(C)c2cc(N(c3ccc4oc5ccccc5c4c3)c3ccccc3-c3ccccc3)ccc2-c2oc3c(c21)C(C)(C)c1cc(N(c2ccc4oc5ccccc5c4c2)c2ccccc2-c2ccccc2)ccc1-3]

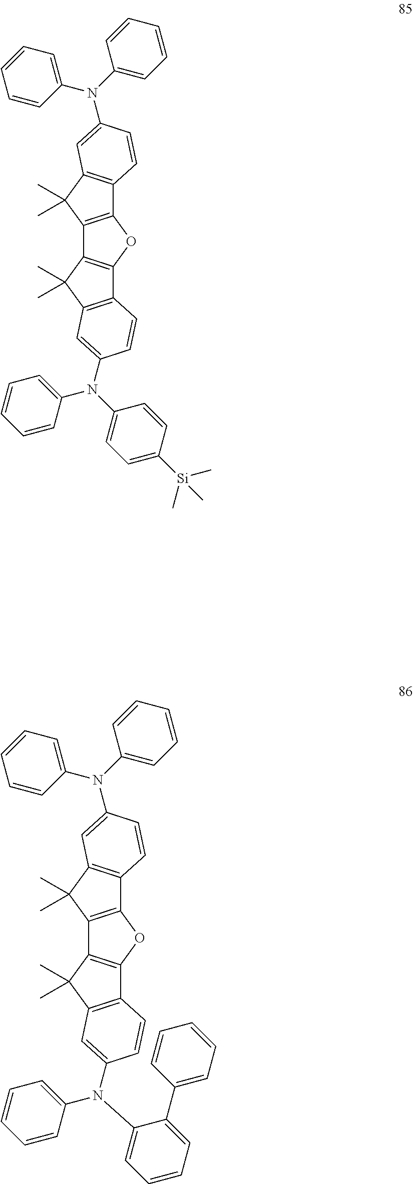 [SMILES: CC1(C)c2cc(N(c3ccccc3)c3ccccc3)ccc2-c2oc3c(c21)C(C)(C)c1cc(N(c2ccccc2)c2ccc([Si](C)(C)C)cc2)ccc1-3 ; CC1(C)c2cc(N(c3ccccc3)c3ccccc3)ccc2-c2oc3c(c21)C(C)(C)c1cc(N(c2ccccc2)c2ccccc2-c2ccccc2)ccc1-3]

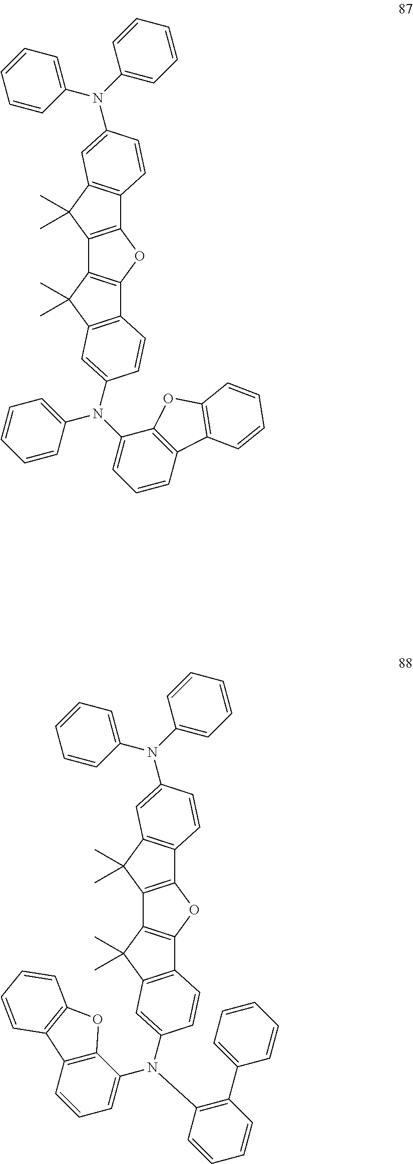 [SMILES: CC1(C)c2cc(N(c3ccccc3)c3ccccc3)ccc2-c2oc3c(c21)C(C)(C)c1cc(N(c2ccccc2)c2cccc4c2oc2ccccc24)ccc1-3 ; CC1(C)c2cc(N(c3ccccc3)c3ccccc3)ccc2-c2oc3c(c21)C(C)(C)c1cc(N(c2ccccc2-c2ccccc2)c2cccc4c2oc2ccccc24)ccc1-3]

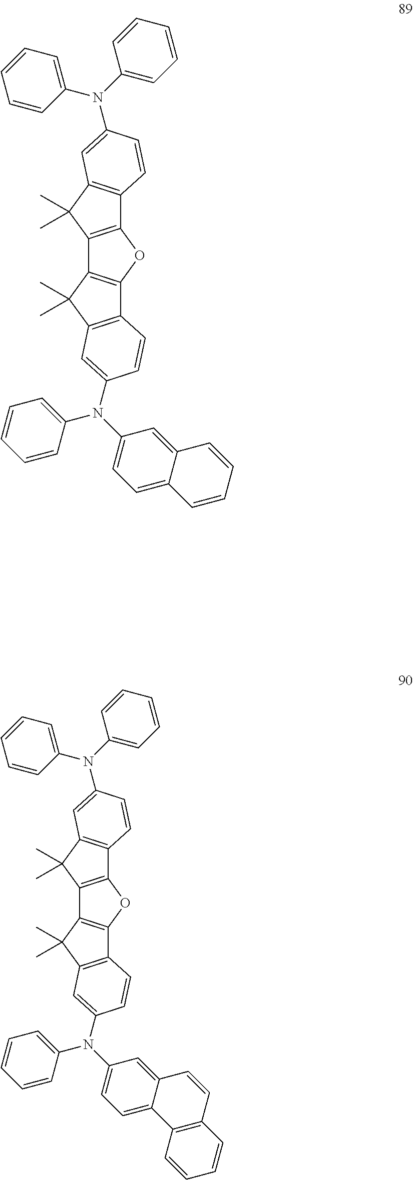 [SMILES: CC1(C)c2cc(N(c3ccccc3)c3ccccc3)ccc2-c2oc3c(c21)C(C)(C)c1cc(N(c2ccccc2)c2ccc4ccccc4c2)ccc1-3 ; CC1(C)c2cc(N(c3ccccc3)c3ccccc3)ccc2-c2oc3c(c21)C(C)(C)c1cc(N(c2ccccc2)c2ccc4c(ccc5ccccc54)c2)ccc1-3]

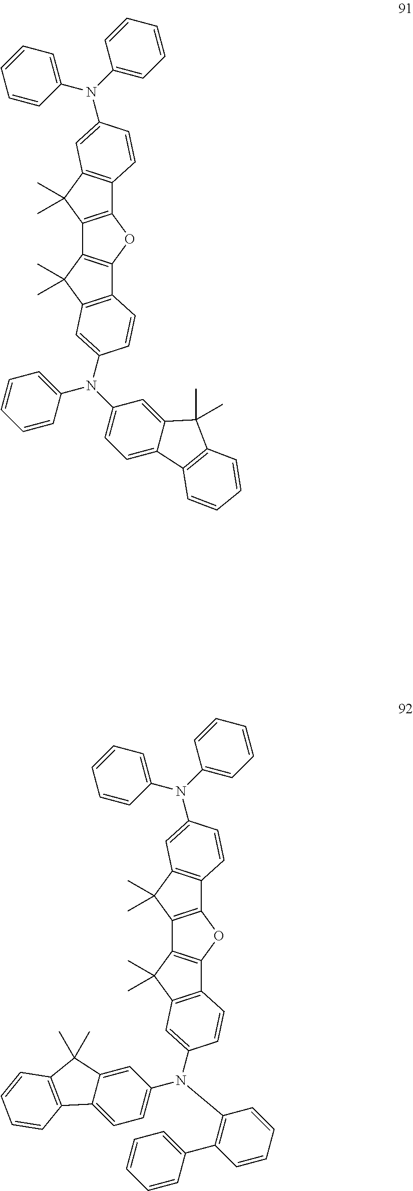 [SMILES: CC1(C)c2ccccc2-c2ccc(N(c3ccccc3)c3ccc4c(c3)C(C)(C)c3c-4oc4c3C(C)(C)c3cc(N(c5ccccc5)c5ccccc5)ccc3-4)cc21 ; CC1(C)c2ccccc2-c2ccc(N(c3ccc4c(c3)C(C)(C)c3c-4oc4c3C(C)(C)c3cc(N(c5ccccc5)c5ccccc5)ccc3-4)c3ccccc3-c3ccccc3)cc21]

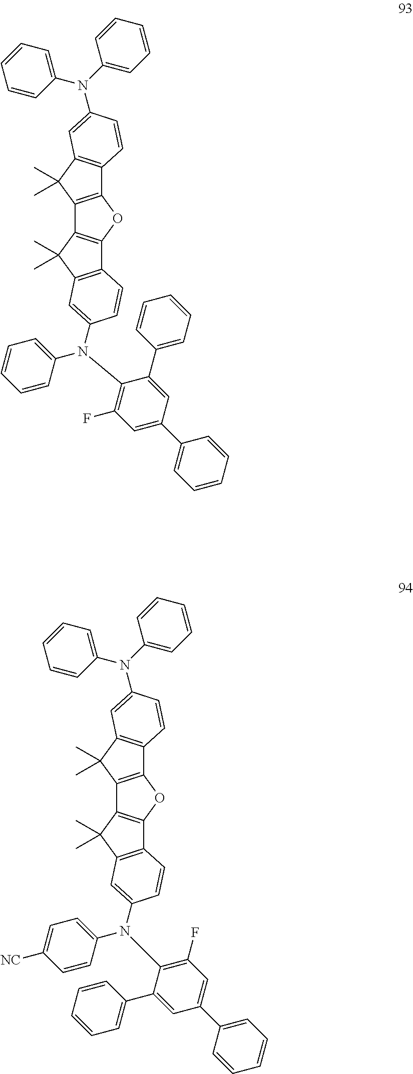 [SMILES: CC1(C)c2cc(N(c3ccccc3)c3ccccc3)ccc2-c2oc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccccc3)ccc2-c2oc3c(c21)C(C)(C)c1cc(N(c2ccc(C#N)cc2)c2c(F)cc(-c4ccccc4)cc2-c2ccccc2)ccc1-3]

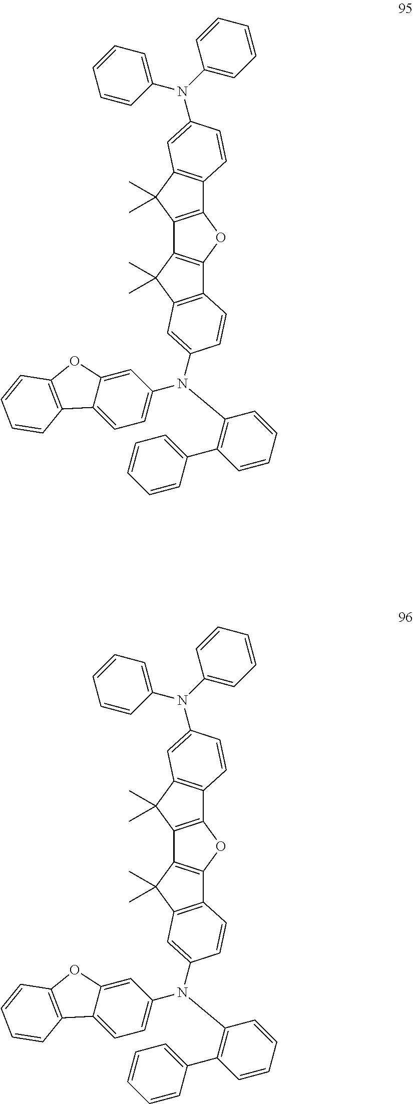 [SMILES: CC1(C)c2cc(N(c3ccccc3)c3ccccc3)ccc2-c2oc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccccc3)ccc2-c2oc3c(c21)C(C)(C)c1cc(N(c2ccc4c(c2)oc2ccccc24)c2ccccc2-c2ccccc2)ccc1-3]

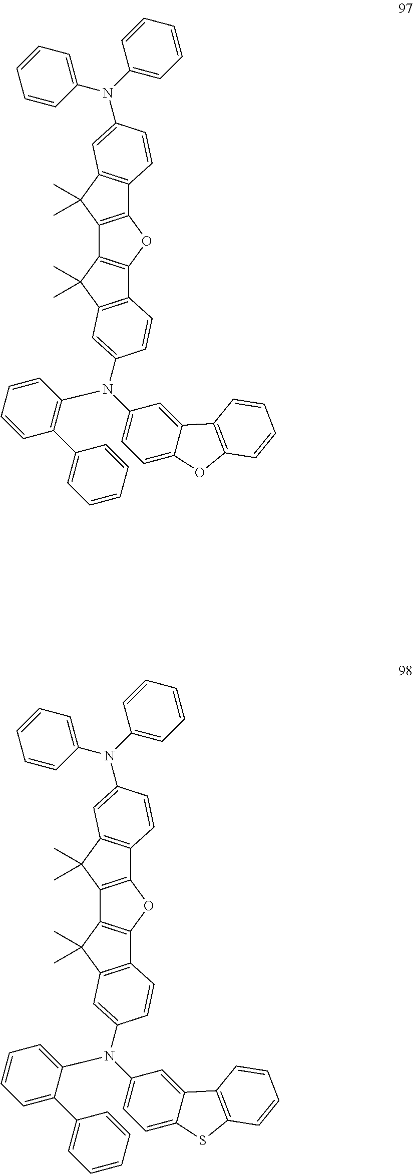 [SMILES: CC1(C)c2cc(N(c3ccccc3)c3ccccc3)ccc2-c2oc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2oc3c(c21)C(C)(C)c1cc(N(c2ccc4sc5ccccc5c4c2)c2ccccc2-c2ccccc2)ccc1-3]

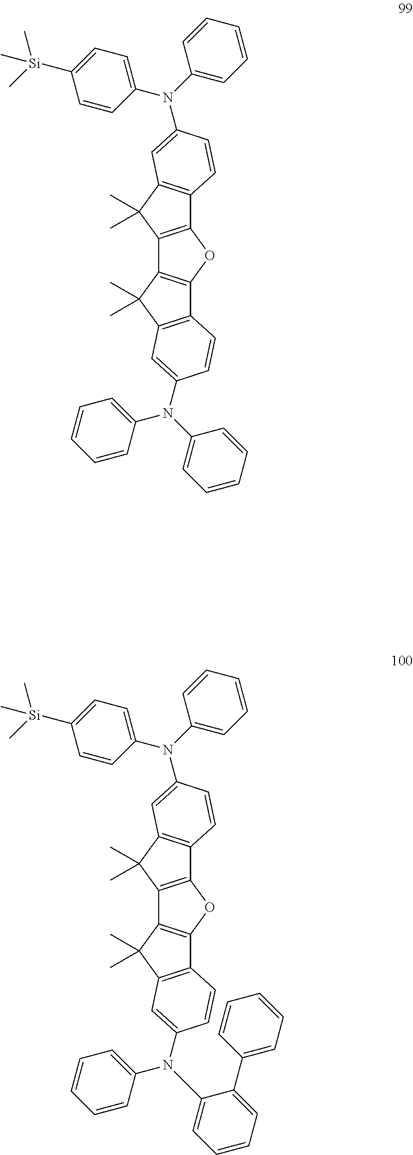 [SMILES: CC1(C)c2cc(N(c3ccccc3)c3ccccc3)ccc2-c2oc3c(c21)C(C)(C)c1cc(N(c2ccccc2)c2ccc([Si](C)(C)C)cc2)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2ccccc2-c2ccccc2)ccc1-3]

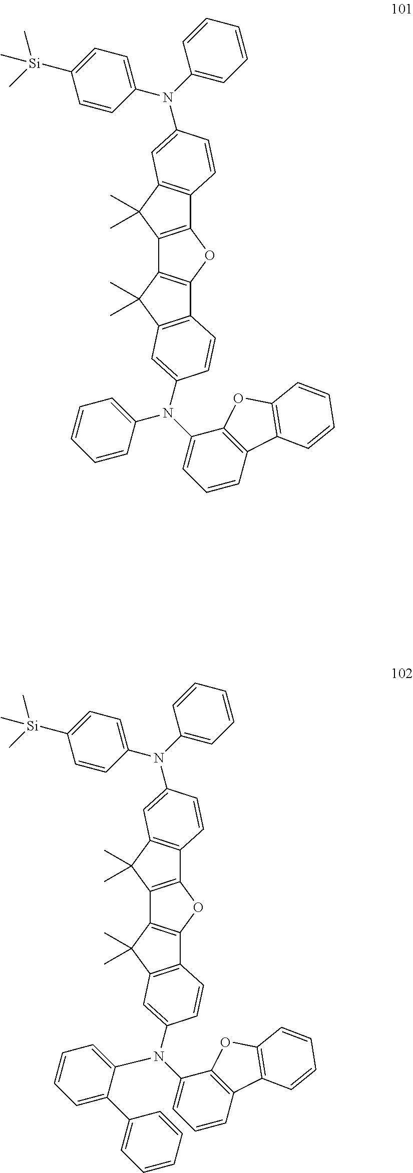 [SMILES: CC1(C)c2cc(N(c3ccccc3)c3ccc([Si](C)(C)C)cc3)ccc2-c2oc3c(c21)C(C)(C)c1cc(N(c2ccccc2)c2cccc4c2oc2ccccc24)ccc1-3 ; CC1(C)c2cc(N(c3ccccc3)c3ccc([Si](C)(C)C)cc3)ccc2-c2oc3c(c21)C(C)(C)c1cc(N(c2ccccc2-c2ccccc2)c2cccc4c2oc2ccccc24)ccc1-3]

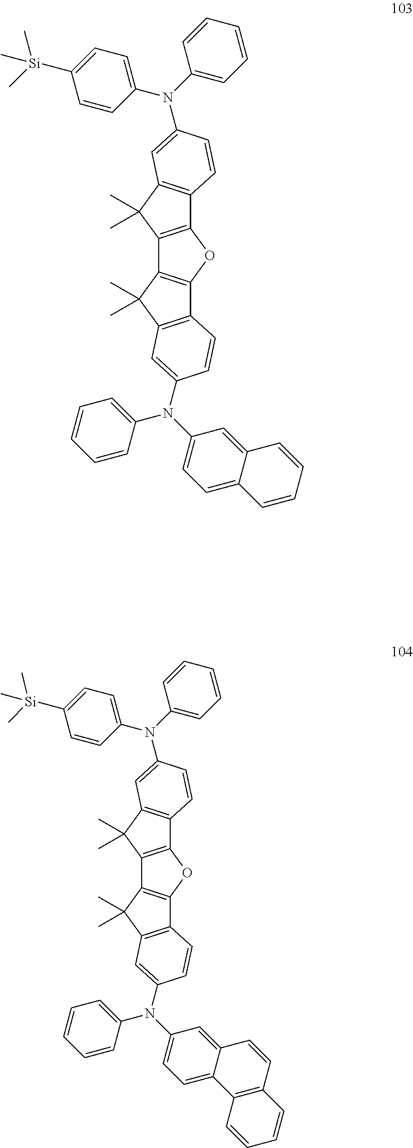 [SMILES: CC1(C)c2cc(N(c3ccccc3)c3ccc([Si](C)(C)C)cc3)ccc2-c2oc3c(c21)C(C)(C)c1cc(N(c2ccccc2)c2ccc4ccccc4c2)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2ccc4c(ccc5ccccc54)c2)ccc1-3]

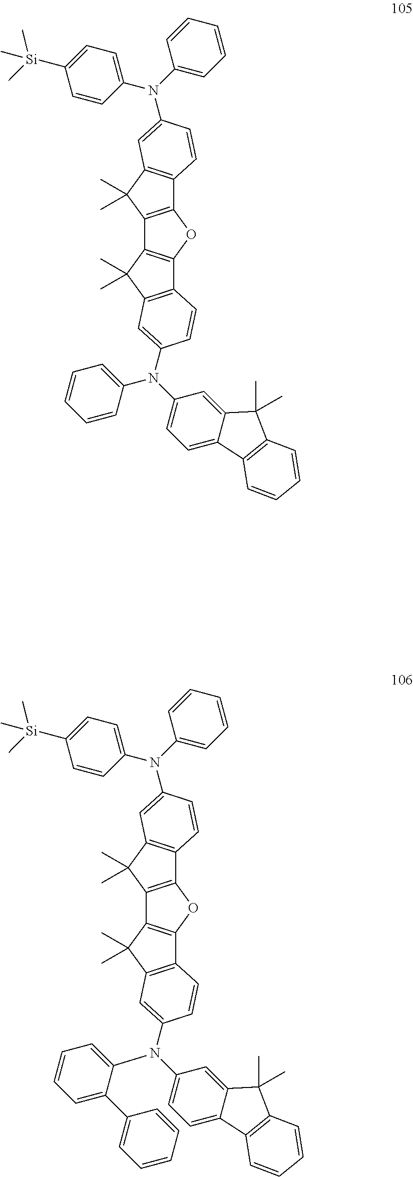 [SMILES: CC1(C)c2ccccc2-c2ccc(N(c3ccccc3)c3ccc4c(c3)C(C)(C)c3c-4oc4c3C(C)(C)c3cc(N(c5ccccc5)c5ccc([Si](C)(C)C)cc5)ccc3-4)cc21 ; CC1(C)c2ccccc2-c2ccc(N(c3ccc4c(c3)C(C)(C)c3c-4oc4c3C(C)(C)c3cc(N(c5ccccc5)c5ccc([Si](C)(C)C)cc5)ccc3-4)c3ccccc3-c3ccccc3)cc21]

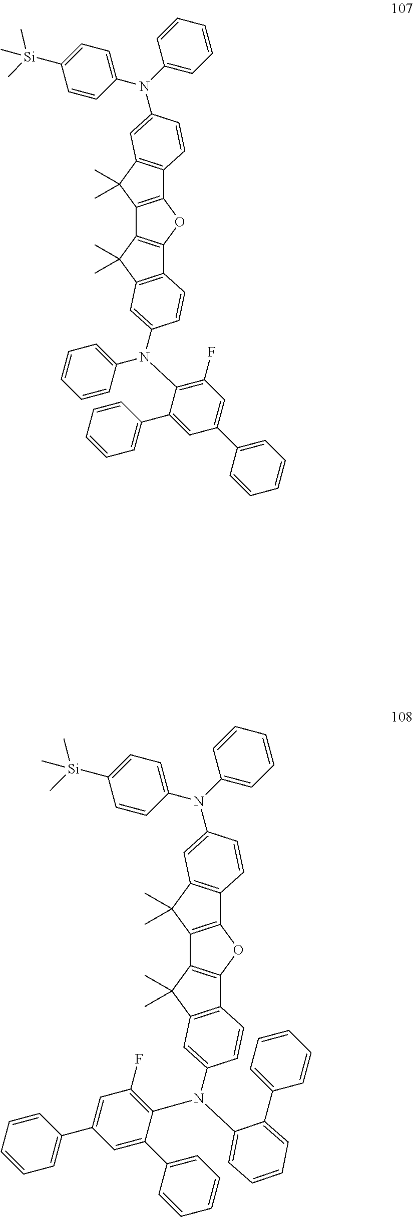 [SMILES: CC1(C)c2cc(N(c3ccccc3)c3ccc([Si](C)(C)C)cc3)ccc2-c2oc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccc([Si](C)(C)C)cc3)ccc2-c2oc3c(c21)C(C)(C)c1cc(N(c2ccccc2-c2ccccc2)c2c(F)cc(-c4ccccc4)cc2-c2ccccc2)ccc1-3]

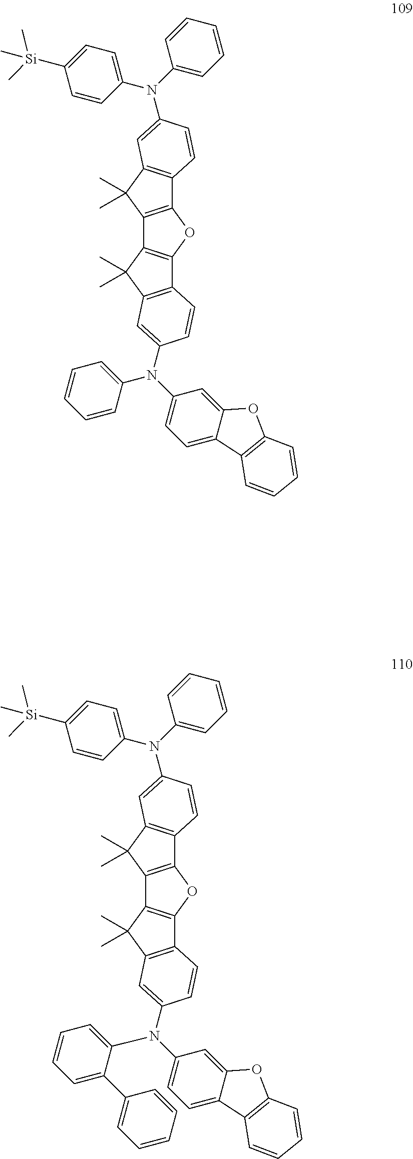 [SMILES: CC1(C)c2cc(N(c3ccccc3)c3ccc([Si](C)(C)C)cc3)ccc2-c2oc3c(c21)C(C)(C)c1cc(N(c2ccccc2)c2ccc4c(c2)oc2ccccc24)ccc1-3 ; CC1(C)c2cc(N(c3ccccc3)c3ccc([Si](C)(C)C)cc3)ccc2-c2oc3c(c21)C(C)(C)c1cc(N(c2ccc4c(c2)oc2ccccc24)c2ccccc2-c2ccccc2)ccc1-3]

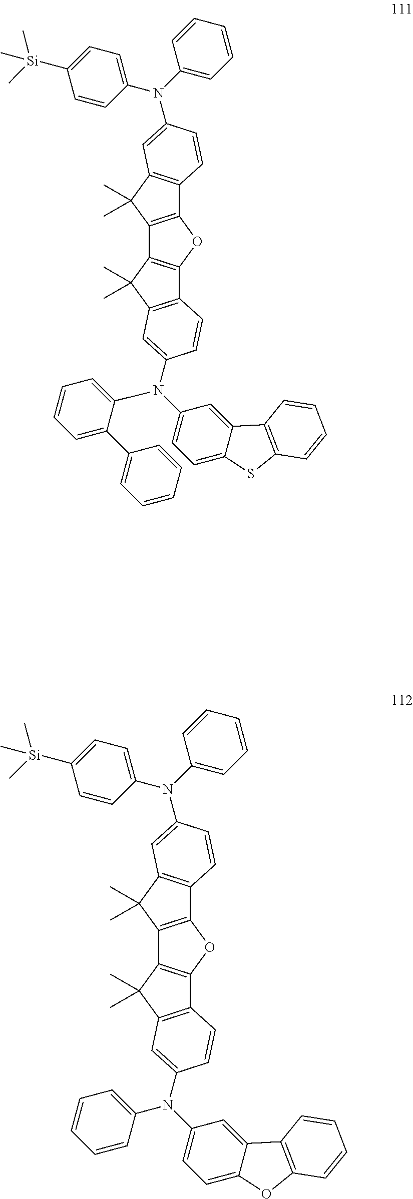 [SMILES: CC1(C)c2cc(N(c3ccccc3)c3ccc([Si](C)(C)C)cc3)ccc2-c2oc3c(c21)C(C)(C)c1cc(N(c2ccc4sc5ccccc5c4c2)c2ccccc2-c2ccccc2)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2ccc4oc5ccccc5c4c2)ccc1-3]

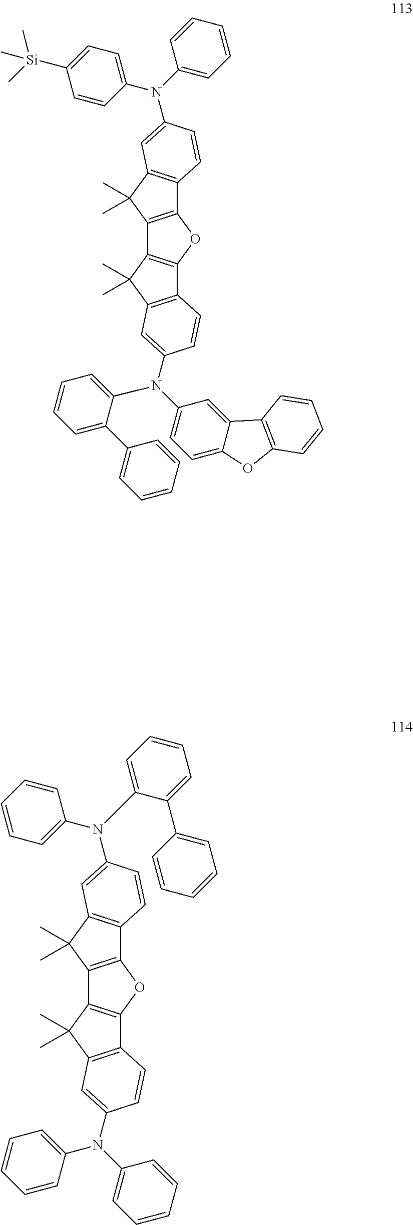 [SMILES: CC1(C)c2cc(N(c3ccccc3)c3ccc([Si](C)(C)C)cc3)ccc2-c2oc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2oc3c(c21)C(C)(C)c1cc(N(c2ccccc2)c2ccccc2-c2ccccc2)ccc1-3]

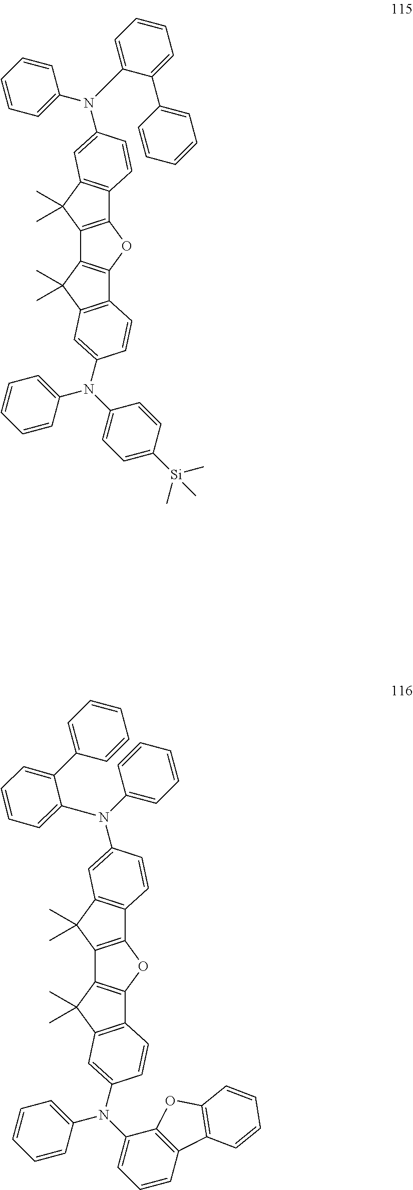 [SMILES: CC1(C)c2cc(N(c3ccccc3)c3ccc([Si](C)(C)C)cc3)ccc2-c2oc3c(c21)C(C)(C)c1cc(N(c2ccccc2)c2ccccc2-c2ccccc2)ccc1-3 ; CC1(C)c2cc(N(c3ccccc3)c3ccccc3-c3ccccc3)ccc2-c2oc3c(c21)C(C)(C)c1cc(N(c2ccccc2)c2cccc4c2oc2ccccc24)ccc1-3]

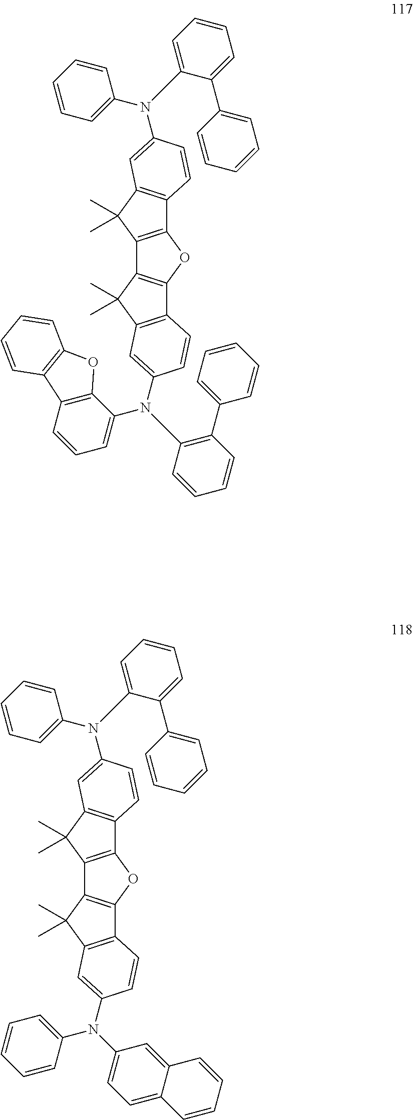 [SMILES: CC1(C)c2cc(N(c3ccccc3)c3ccccc3-c3ccccc3)ccc2-c2oc3c(c21)C(C)(C)c1cc(N(c2ccccc2-c2ccccc2)c2cccc4c2oc2ccccc24)ccc1-3 ; CC1(C)c2cc(N(c3ccccc3)c3ccc4ccccc4c3)ccc2-c2oc3c(c21)C(C)(C)c1cc(N(c2ccccc2)c2ccccc2-c2ccccc2)ccc1-3]

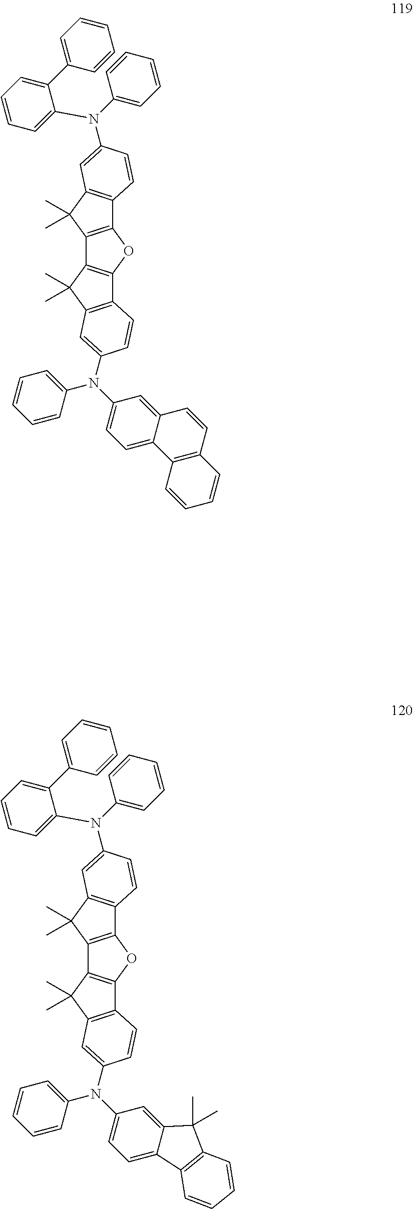 [SMILES: CC1(C)c2cc(N(c3ccccc3)c3ccc4c(ccc5ccccc54)c3)ccc2-c2oc3c(c21)C(C)(C)c1cc(N(c2ccccc2)c2ccccc2-c2ccccc2)ccc1-3 ; CC1(C)c2ccccc2-c2ccc(N(c3ccccc3)c3ccc4c(c3)C(C)(C)c3c-4oc4c3C(C)(C)c3cc(N(c5ccccc5)c5ccccc5-c5ccccc5)ccc3-4)cc21]

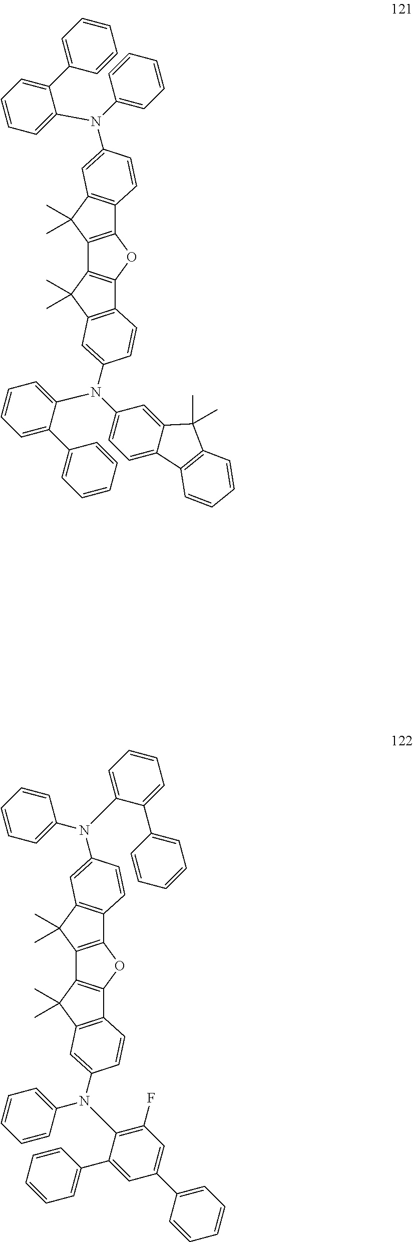 [SMILES: CC1(C)c2ccccc2-c2ccc(N(c3ccc4c(c3)C(C)(C)c3c-4oc4c3C(C)(C)c3cc(N(c5ccccc5)c5ccccc5-c5ccccc5)ccc3-4)c3ccccc3-c3ccccc3)cc21 ; CC1(C)c2cc(N(c3ccccc3)c3ccccc3-c3ccccc3)ccc2-c2oc3c(c21)C(C)(C)c1cc(N(c2ccccc2)c2c(F)cc(-c4ccccc4)cc2-c2ccccc2)ccc1-3]

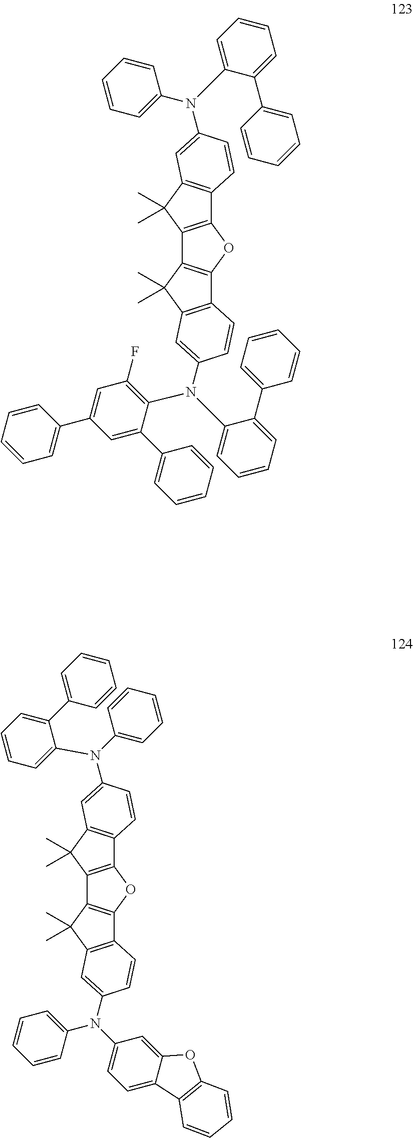 [SMILES: CC1(C)c2cc(N(c3ccccc3)c3ccccc3-c3ccccc3)ccc2-c2oc3c(c21)C(C)(C)c1cc(N(c2ccccc2-c2ccccc2)c2c(F)cc(-c4ccccc4)cc2-c2ccccc2)ccc1-3 ; CC1(C)c2cc(N(c3ccccc3)c3ccc4c(c3)oc3ccccc34)ccc2-c2oc3c(c21)C(C)(C)c1cc(N(c2ccccc2)c2ccccc2-c2ccccc2)ccc1-3]

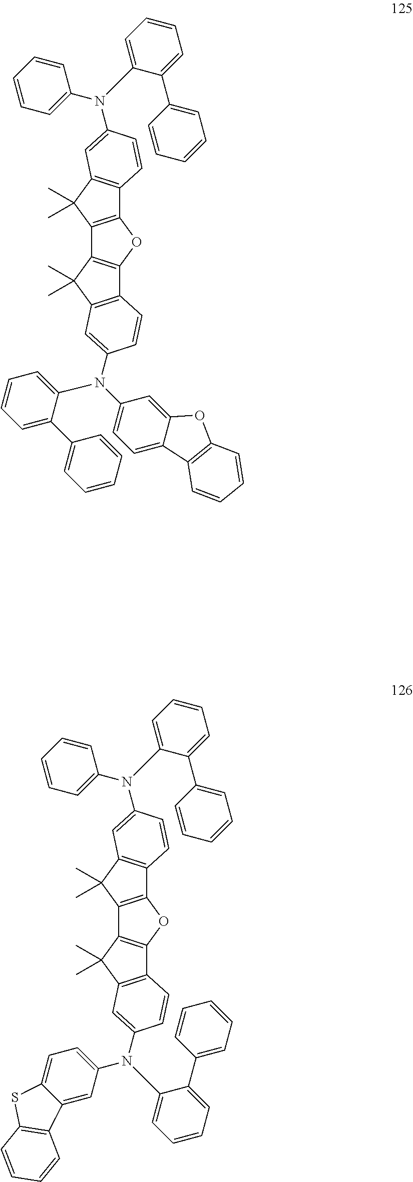 [SMILES: CC1(C)c2cc(N(c3ccccc3)c3ccccc3-c3ccccc3)ccc2-c2oc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccccc3-c3ccccc3)ccc2-c2oc3c(c21)C(C)(C)c1cc(N(c2ccc4sc5ccccc5c4c2)c2ccccc2-c2ccccc2)ccc1-3]

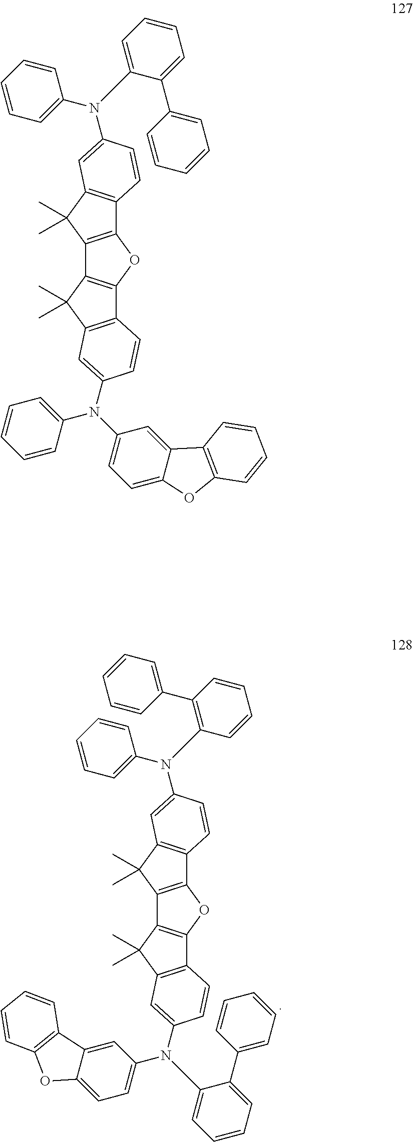 [SMILES: CC1(C)c2cc(N(c3ccccc3)c3ccc4oc5ccccc5c4c3)ccc2-c2oc3c(c21)C(C)(C)c1cc(N(c2ccccc2)c2ccccc2-c2ccccc2)ccc1-3 ; CC1(C)c2cc(N(c3ccccc3)c3ccccc3-c3ccccc3)ccc2-c2oc3c(c21)C(C)(C)c1cc(N(c2ccc4oc5ccccc5c4c2)c2ccccc2-c2ccccc2)ccc1-3]

The term “organic layer” as used herein refers to a single layer and/or a plurality of layers disposed between the first electrode and the second electrode of the organic light-emitting device. A material included in the “organic layer,” however, is not limited to an organic compound. For example, the “organic layer” may include an inorganic compound.

The accompanying drawing illustrates a schematic cross-sectional view of an organic light-emitting device 10 according to an example embodiment. The organic light-emitting device 10 has a structure of a first electrode 110, an organic layer 150, and a second electrode 190.

Hereinafter, a structure of an organic light-emitting device according to an example embodiment and a method of manufacturing an organic light-emitting device according to an example embodiment will be described in connection with the accompanying drawing.

In the embodiment shown in the accompanying drawing, a substrate may be additionally disposed under the first electrode 110 and/or above the second electrode 190. The substrate may be a glass substrate or a transparent plastic substrate, each with excellent mechanical strength, thermal stability, transparency, surface smoothness, ease of handling, and water repellency.

The first electrode 110 may be formed by, for example, depositing or sputtering a material for forming the first electrode 110 on the substrate. When the first electrode 110 is an anode, the material for forming the first electrode 110 may be selected from materials having a high work function to facilitate hole injection. The first electrode 110 may be a reflective electrode, a semi-transmissive electrode, or a transmissive electrode. The material for forming the first electrode 110 may be indium tin oxide (ITO), indium zinc oxide (IZO), tin oxide (SnO₂), or zinc oxide (ZnO), each having transparency and excellent conductivity. In some embodiments, when the first electrode 110 is a semi-transparent electrode or a reflective electrode, at least one selected from magnesium (Mg), aluminum (Al), aluminum-lithium (Al—Li), calcium (Ca), magnesium-indium (Mg—In), and magnesium-silver (Mg—Ag) may be utilized as a material for forming the first electrode 110.

The first electrode 110 may have a single-layer structure or a multi-layer structure including a plurality of layers. For example, the first electrode 110 may have a triple-layered structure of ITO/Ag/ITO, but the structure of the first electrode 110 is not limited thereto.

An organic layer 150 may be disposed on top of the first electrode 110, and may include an emission layer.

The organic layer 150 may further include a hole transport region disposed between the first electrode 110 and the emission layer, and an electron transport region disposed between the emission layer and the second electrode 190.

The hole transport region may include at least one selected from a hole transport layer (HTL), a hole injection layer (HIL), a buffer layer, and an electron blocking layer, and the electron transport region may include at least one selected from a hole blocking layer HBL, an electron transport layer ETL, and an electron injection layer EIL, but the hole transport region and the electron transport region are not limited thereto.

The hole transport region may have a single-layered structure including a single material, a single-layered structure including a plurality of different materials, or a multi-layered structure including a plurality of layers formed of a plurality of different materials.

For example, the hole transport region may have a single-layered structure including a plurality of different materials, or a structure of HIL/HTL, a structure of HIL/HTL/buffer layer, a structure of HIL/buffer layer, a structure of HTL/buffer layer, or a structure of HIL/HTL/EBL. In the foregoing structures, layers of each structure are sequentially stacked in the stated order from the first electrode 110, but the hole transport region is not limited thereto.

When the hole transport region includes an HIL, the HIL may be formed on the first electrode 110 by utilizing one or more suitable methods, such as vacuum deposition, spin coating, casting, a Langmuir-Blodgett (LB) method, ink-jet printing, laser-printing, or a laser-induced thermal imaging (LITI) method.

When an HIL is formed by vacuum deposition, the vacuum deposition may be performed, for example, at a deposition temperature of about 100° C. to about 500° C., at a vacuum degree of about 10⁻⁸ torr to about 10⁻³ torr, and at a deposition rate of about 0.01 Å/sec to about 100 Å/sec, in consideration of a compound for forming the HIL to be deposited and a structure of the HIL to be formed (e.g., in view of the characteristics of the compound being deposited and the characteristics of the HIL being formed).

When an HIL is formed by spin coating, the coating may be performed, for example, at a coating speed of about 2,000 rpm to about 5,000 rpm and at a temperature of about 80° C. to about 200° C., in consideration of a compound for forming the HIL to be deposited and a structure of the HIL to be formed (e.g., in view of the characteristics of the compound being coated and the characteristics of the HIL being formed).

When the hole transport region includes an HTL, the HTL may be formed on the first electrode 110 or the HIL by utilizing one or more suitable methods, such as vacuum deposition, spin coating, casting, an LB method, an ink-jet printing, a laser-printing, or an LITI method. When the HTL is formed by vacuum deposition and/or spin coating, the deposition and coating conditions for the HTL may be determined by referring to the deposition and coating conditions for the HIL.

The hole transport region may include at least one selected from m-MTDATA, TDATA, 2-TNATA, NPB, β-NPB, TPD, Spiro-TPD, Spiro-NPB, α-NPB, TAPC, HMTPD, 4,4′,4″-tris(N-carbazolyl)triphenylamine (TCTA), polyaniline/dodecylbenzenesulfonic acid (Pani/DBSA), poly(3,4-ethylenedioxythiophene)/poly(4-styrenesulfonate) (PEDOT/PSS), polyaniline/camphor sulfonic acid (Pani/CSA), polyaniline/poly(4-styrenesulfonate) (PANI/PSS), a compound represented by Formula 201, and a compound represented by Formula 202:

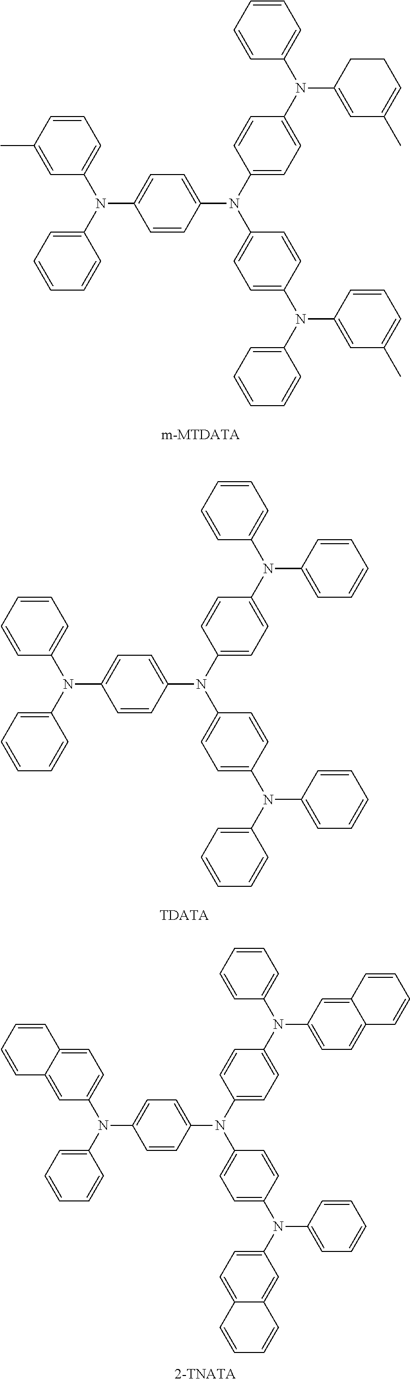

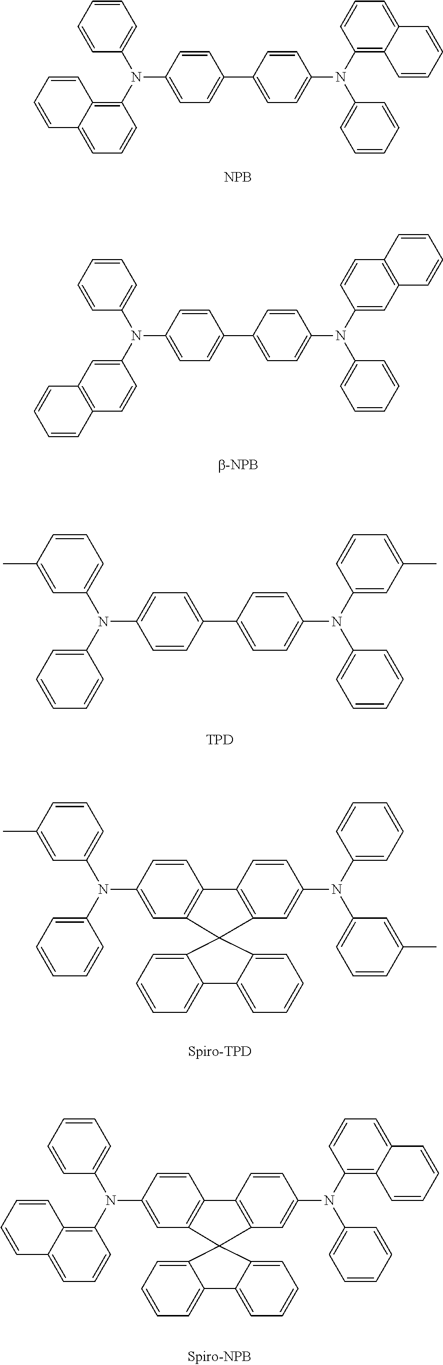

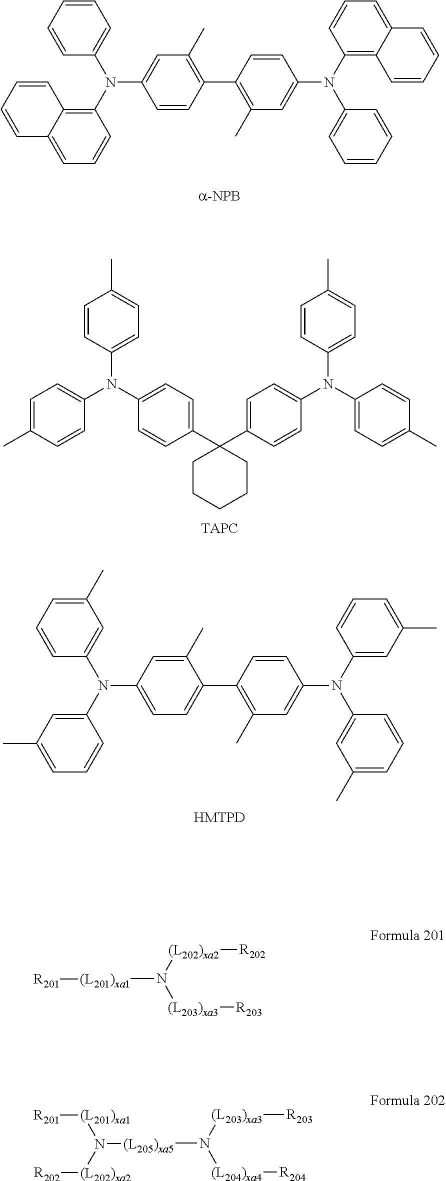

In Formulae 201 and 202,

xa1 to xa4 may each be independently selected from 0, 1, 2, and 3,

xa5 may be selected from 1, 2, 3, 4, and 5, and

R₂₀₁ to R₂₀₄ may each be independently selected from a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, in Formulae 201 and 202,

L₂₀₁ to L₂₀₅ may each be independently selected from:

a phenylene group, a naphthylene group, a fluorenylene group, a spiro-fluorenylene group, a benzofluorene group, a dibenzofluorene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, an isoindolyl group, a quinolinyl group, an isoquinolinyl group, a quinoxalinyl group, a quinazolinyl group, a carbazolyl group, and a triazinyl group,

xa1 to xa4 may each be independently 0, 1, or 2,

xa5 may be 1, 2, or 3, and

R₂₀₁ to R₂₀₄ may each be independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an azulenyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group.

The compound represented by Formula 201 may be represented by Formula 201A:

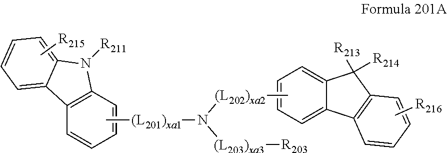

For example, the compound represented by Formula 201 may be represented by Formula 201A-1, but the compound is not limited thereto:

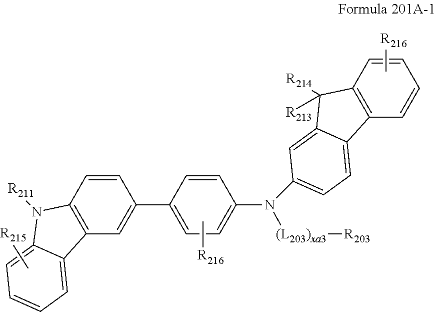

The compound represented by Formula 202 may be represented by Formula 202A, but the compound is not limited thereto:

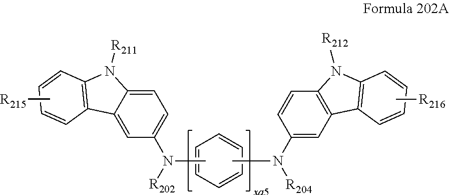

In Formulae 201A, 201A-1, and 202A, L₂₀₁ to L₂₀₃, xa1 to xa3, xa5, and R₂₀₂ to R₂₀₄ may be understood by referring to the descriptions provided in the present specification, R₂₁₁ and R₂₁₂ may be understood by referring to the descriptions provided in connection with R₂₀₃, and R₂₁₃ to R₂₁₆ may each be independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀aryloxy group, a C₆-C₆₀arylthio group, a C₁-C₆₀ heteroaryl group, a monovalent non-aromatic condensed polycyclic group, and a monovalent non-aromatic condensed heteropolycyclic group.

For example, in Formulae 201A, 201A-1, and 202A,

L₂₀₁ to L₂₀₃ may each be independently selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, a chrysenylene group, a pyridinylene group, a pyrazinylene group, a pyrimidinylene group, a pyridazinylene group, a quinolinylene group, an isoquinolinylene group, a quinoxalinylene group, a quinazolinylene group, a carbazolylene group, and a triazinylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

xa1 to xa3 may each be independently 0 or 1,

R₂₀₃, R₂₁₁, and R₂₁₂ may each be independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, carboxylic acid a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

R₂₁₃ and R₂₁₄ may each be independently selected from:

a C₁-C₂₀ alkyl group and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

R₂₁₅ and R₂₁₆ may each be independently selected from:

a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, and

xa5 may be 1 or 2.

In Formulae 201A and 201A-1, R₂₁₃ and R₂₁₄ may bind to each other (e.g., combine together) to form a saturated or unsaturated ring.

The compound represented by Formula 201 and the compound represented by Formula 202 may each include at least one selected from Compounds HT1 to HT20 below, but the compounds are not limited thereto.

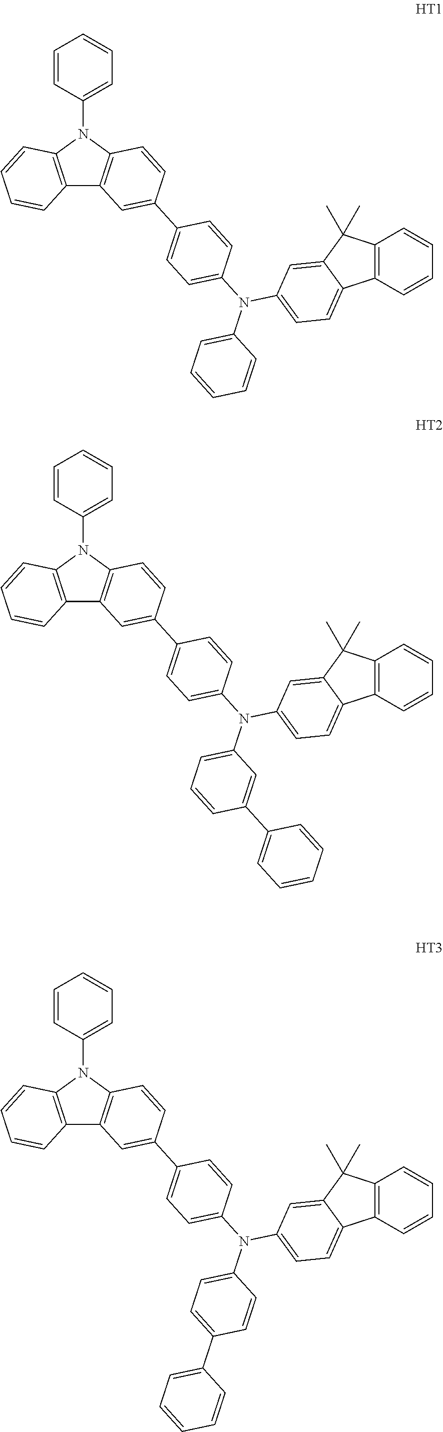 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

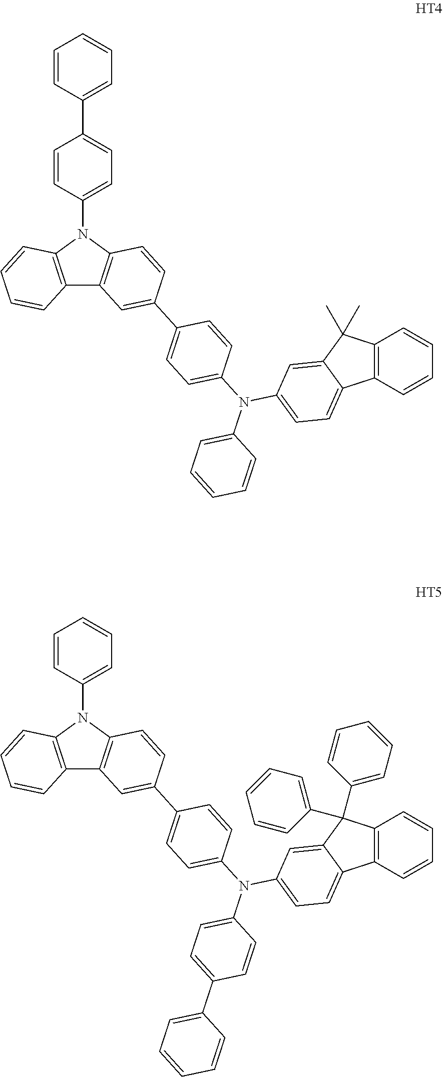 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

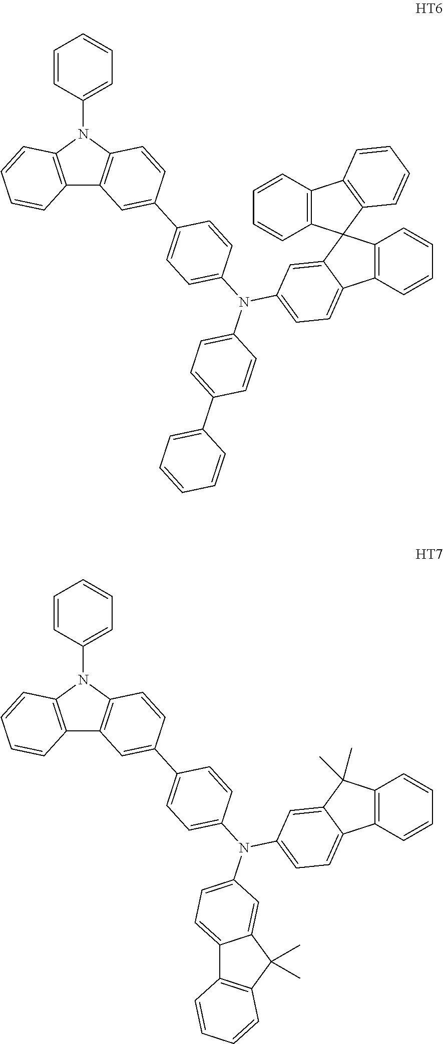 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

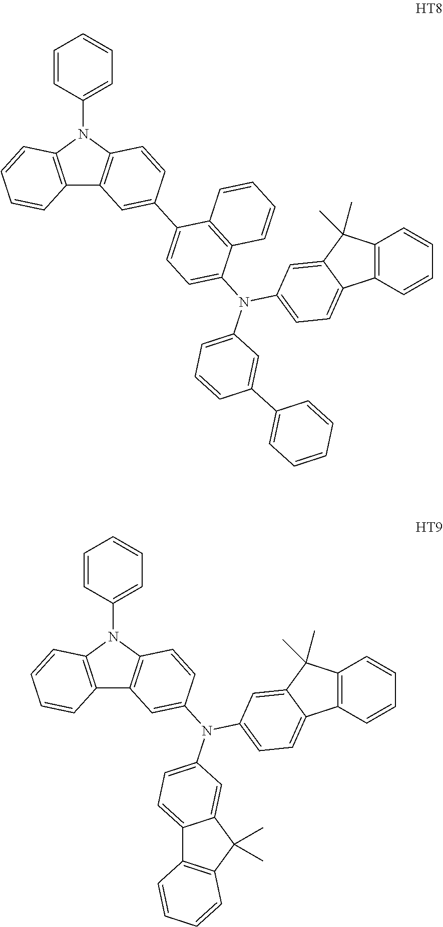 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

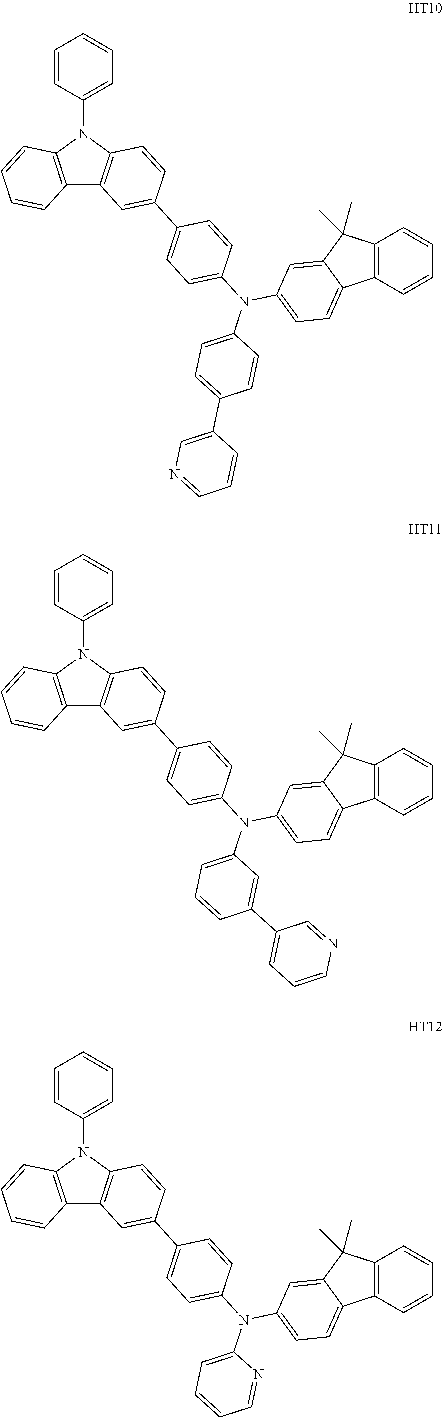 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

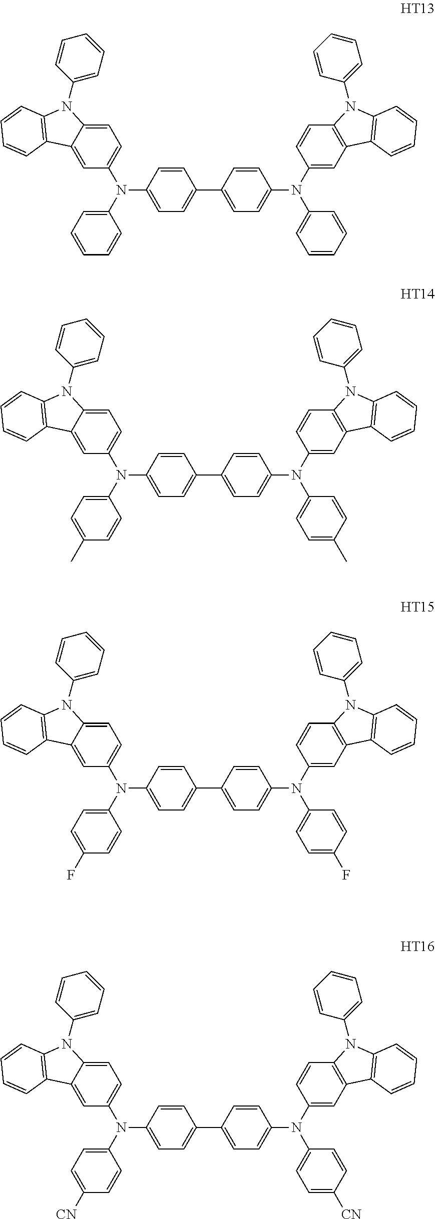

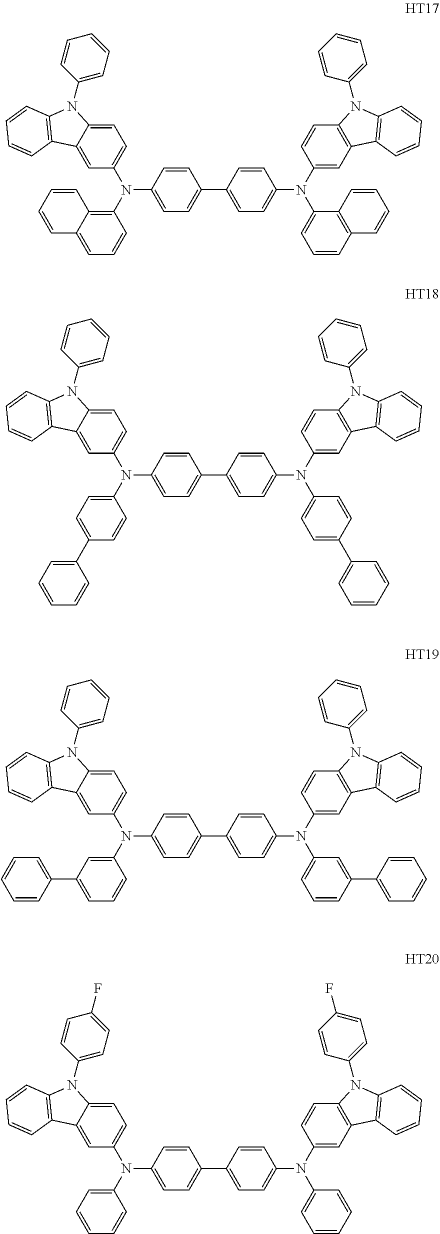 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Å to about 10,000 Å, for example, about 100 Å to about 1,000 Å. When the hole transport region includes both an HIL and an HTL, a thickness of the HIL may be in a range of about 100 Å to about 10,000 Å, for example, about 100 Å to about 1,000 Å, and a thickness of the HTL may be in a range of about 50 Å to about 2,000 Å, for example, about 100 Å to about 1,500 Å. When the thickness of the hole transport region, the HIL, and the HTL are within these ranges, satisfactory or suitable hole transporting characteristics are obtained without a substantial increase in driving voltage.

The hole transport region may further include, in addition to the foregoing materials, a charge-generation material for the improvement of conductive properties. The charge-generation material may be homogeneously or inhomogeneously dispersed in the hole transport region.

The charge-generation material may be, for example, a p-dopant. The p-dopant may be one of (e.g., may be selected from) a quinone derivative, a metal oxide, and a cyano group-containing compound, but the p-dopant is not limited thereto. For example, non-limiting examples of the p-dopant include a quinone derivative such as tetracyanoquinodimethane (TCNQ) and 2,3,5,6-tetrafluoro-tetracyano-1,4-benzoquinodimethane (F4-TCNQ); a metal oxide such as a tungsten oxide and a molybdenum oxide; and Compound HT-D1 below, but the p-dopant is not limited thereto.

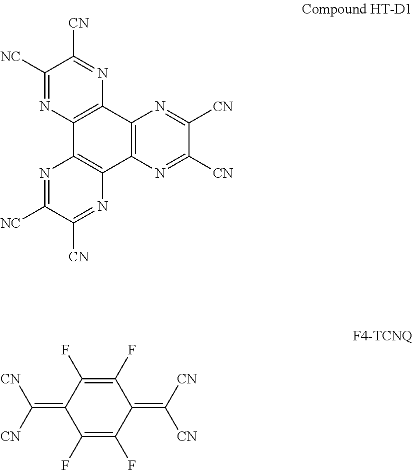

The hole transport region may further include, in addition to the EBL, the HIL, and the HTL, a buffer layer. The buffer layer may compensate for an optical resonance distance according to a wavelength of light emitted from the emission layer, thereby improving light-emission efficiency of a formed organic light-emitting device. For usage as a material included in the buffer layer, materials that are included in the hole transport region may be utilized. The EBL may reduce or prevent injection of electrons from the electron transport region.

The emission layer may be formed on the first electrode 110 or on the hole transport region by utilizing one or more suitable methods, such as vacuum deposition, spin coating, casting, an LB method, an ink-jet printing, a laser-printing, or an LITI method. When the emission layer is formed by vacuum deposition and/or spin coating, the deposition and coating conditions for the emission layer may be determined by referring to the deposition and coating conditions for the HIL.

When the organic light-emitting device 10 is a full color organic light-emitting device, the emission layer may be patterned into a red emission layer, a green emission layer, or a blue emission layer, according to individual sub pixels, respectively. The emission layer may have various suitable modifications in the structure, and for example, may have a stacked structure of a red emission layer, a green emission layer, and a blue emission layer, or a mixed structure of a red light-emitting material, a green light-emitting material, and a blue light-emitting material that are mixed without distinction between layers, and accordingly the emission layer may emit white light.

The emission layer may include a host and a dopant.

The host may include, for example, one selected from TPBi, TBADN, AND (also referred to as “DNA”), CBP, CDBP, and TCP:

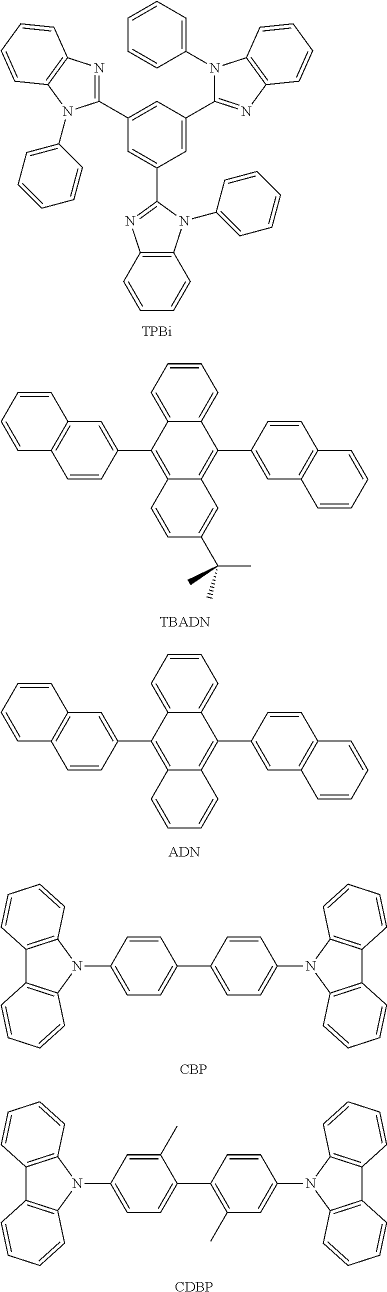

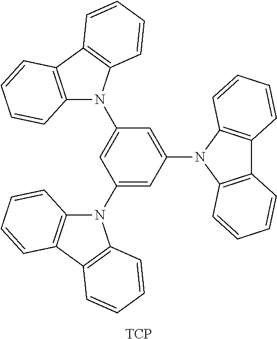

In some embodiments, the host may include a compound represented by Formula 301:

Ar₃₀₁-[(L₃₀₁)_{xb1}-R₃₀₁]_{xb2} Formula 301

In Formula 301,

Ar₃₀₁ may be selected from:

a naphthalene, a heptalene, a fluorenene, a spiro-fluorene, a benzofluorene, a dibenzofluorene, a phenalene, a phenanthrene, an anthracene, a fluoranthene, a triphenylene, a pyrene, a chrysene, a naphthacene, a picene, a perylene, a pentaphene, and an indenoanthracene; and

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, naphthacene, a picene, a perylene, a pentaphene, and an indenoanthracene, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀aryloxy group, a C₆-C₆₀arylthio group, a C₁-C₆₀ heteroaryl group, a monovalent non-aromatic condensed polycyclic group, a monovalent non-aromatic condensed heteropolycyclic group and —Si(Q₃₀₁)(Q₃₀₂)(Q₃₀₃) (wherein Q₃₀₁ to Q₃₀₃ may each be independently selected from a hydrogen, a C₁-C₆₀ alkyl group, a C₂-C₆₀ alkenyl group, a C₆-C₆₀ aryl group, and a C₁-C₆₀ heteroaryl group),

L₃₀₁ may be understood by referring to the descriptions provided in connection with L₂₀₁,

R₃₀₁ may be selected from:

a C₁-C₂₀ alkyl group and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group,

xb1 may be selected from 0, 1, 2, and 3, and

xb2 may be selected from 1, 2, 3, and 4.

For example, in Formula 301,

L₃₀₁ may be selected from:

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, and a chrysenylene group; and

a phenylene group, a naphthylene group, a fluorenylene group, a spiro-fluorenylene group, a benzofluorenylene group, a dibenzofluorenylene group, a phenanthrenylene group, an anthracenylene group, a pyrenylene group, and a chrysenylene group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group, and

R₃₀₁ may be selected from:

a C₁-C₂₀ alkyl group and a C₁-C₂₀ alkoxy group;

a C₁-C₂₀ alkyl group and a C₁-C₂₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group;

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, and a chrysenyl group, but the compound represented by Formula 301 is not limited thereto.

For example, the host may include a compound represented by Formula 301A:

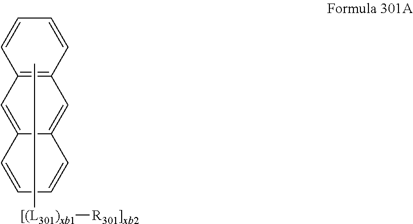

The substituents of Formula 301A may be understood by referring to the descriptions provided in the present specification.

The compound represented by Formula 301 may include at least one selected from Compounds H1 to H42 below, but the compound represented by Formula 301 is not limited thereto:

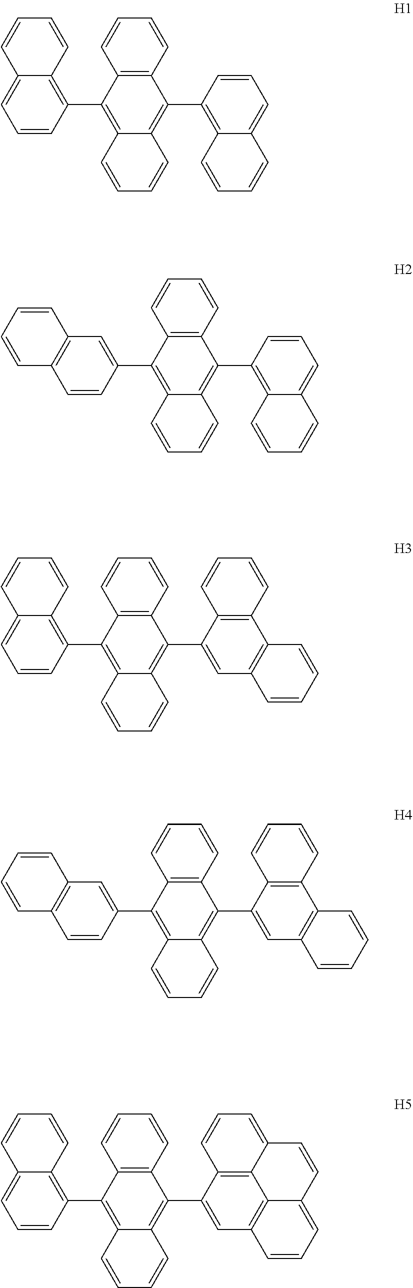

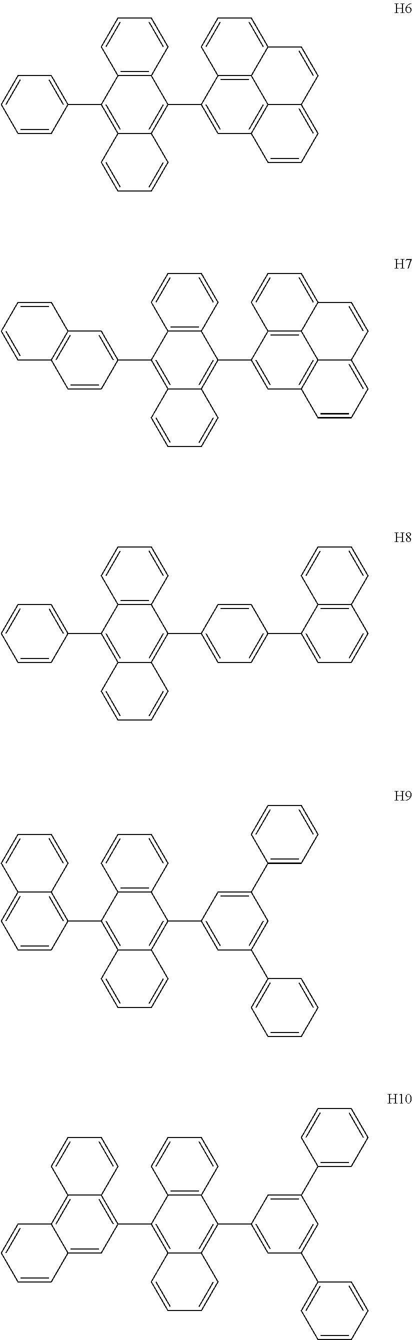

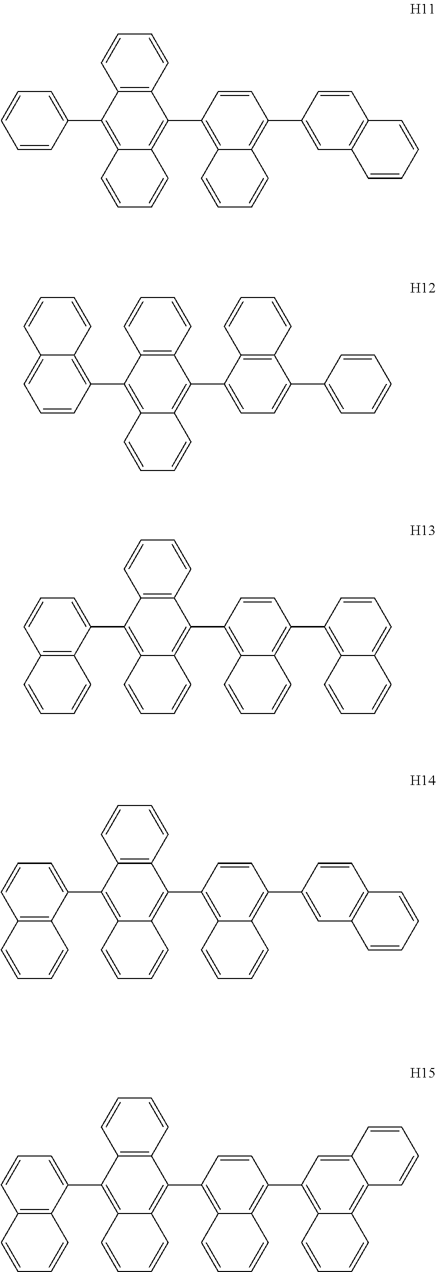

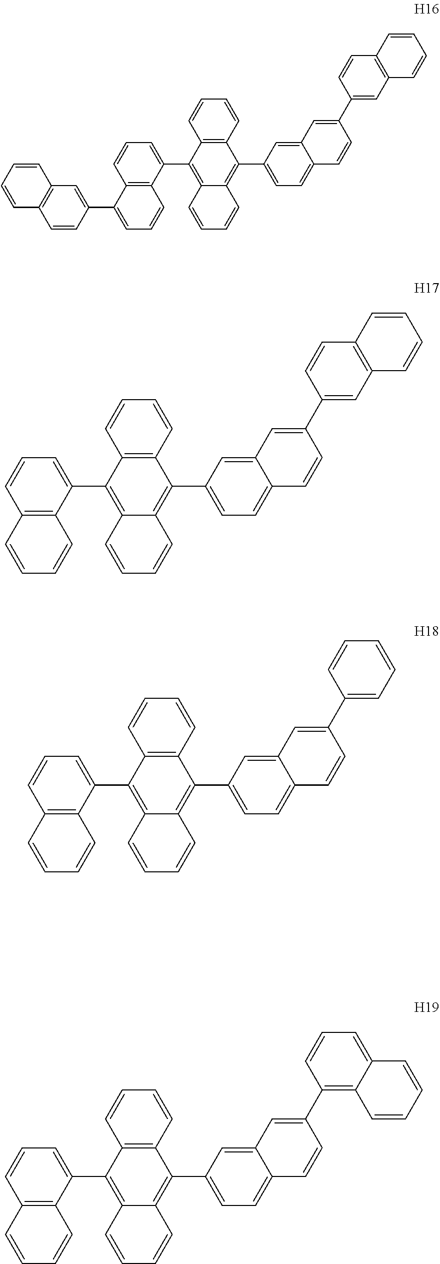

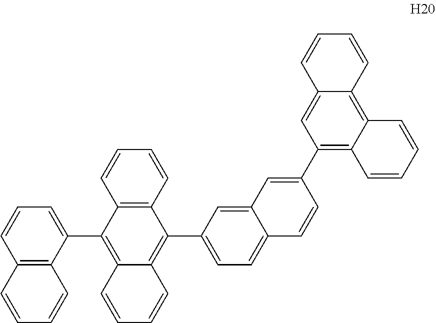 [SMILES: c1ccc2c(-c3c4ccccc4c(-c4ccc5ccc(-c6cc7ccccc7c7ccccc67)cc5c4)c4ccccc34)cccc2c1]

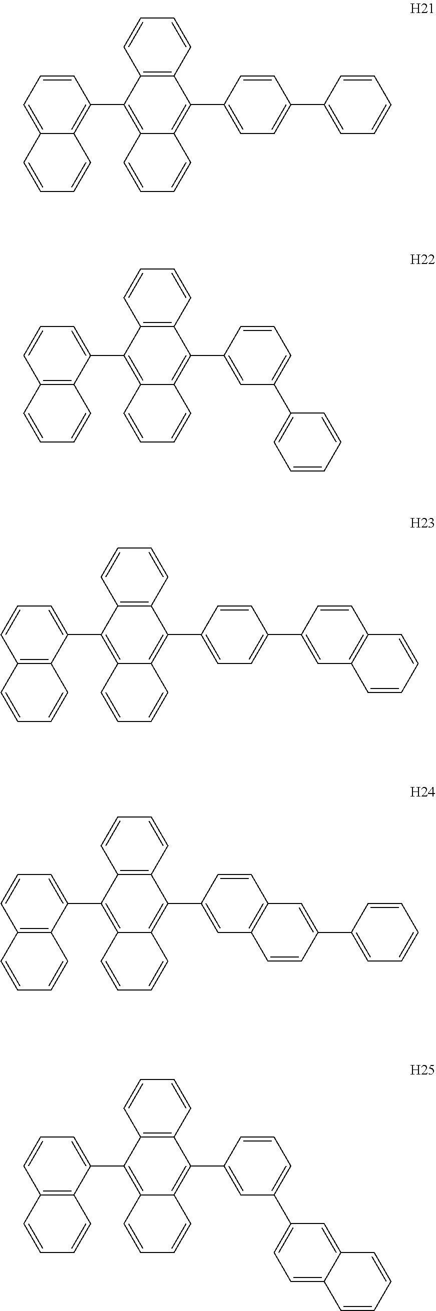

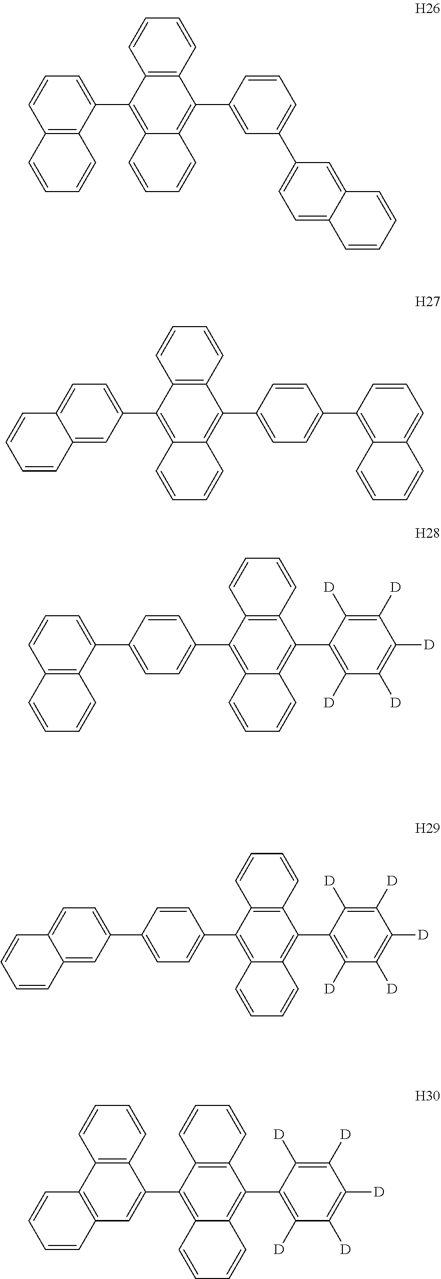

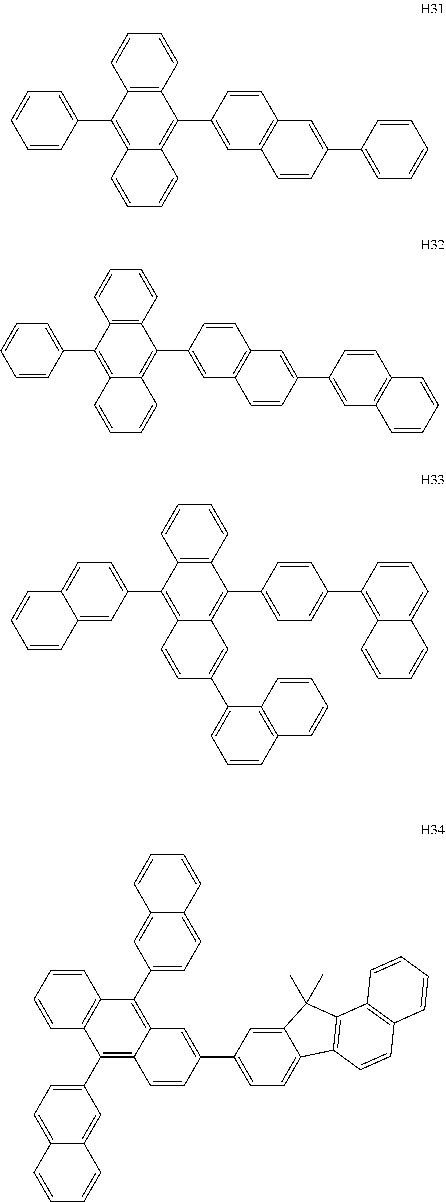

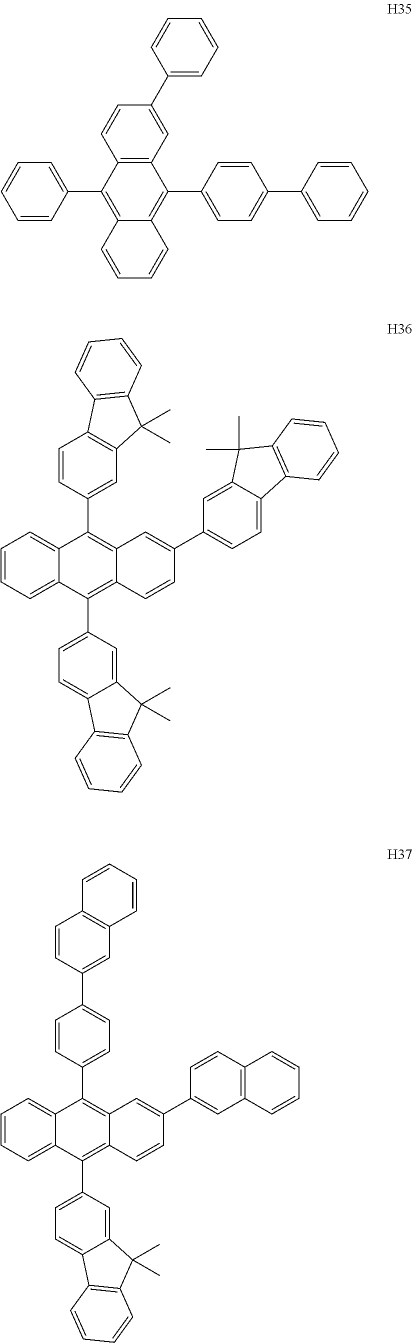

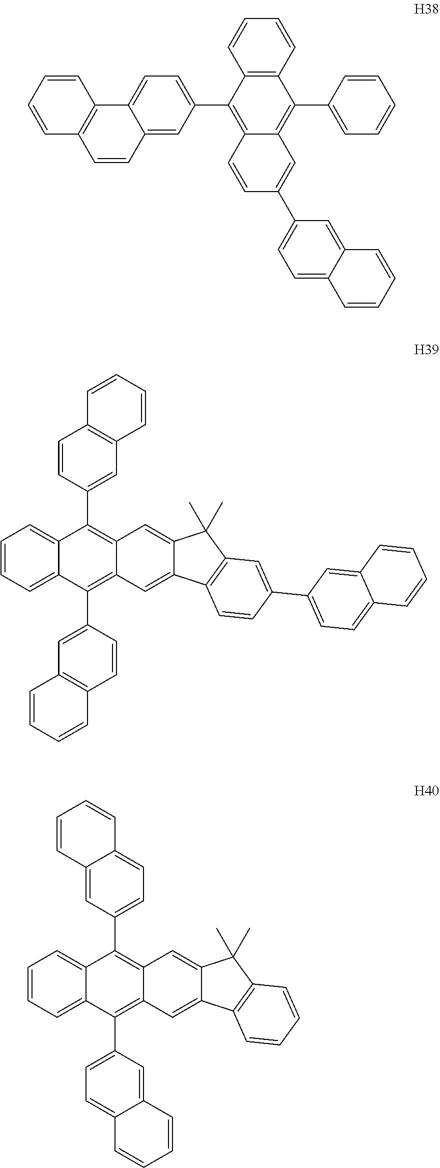

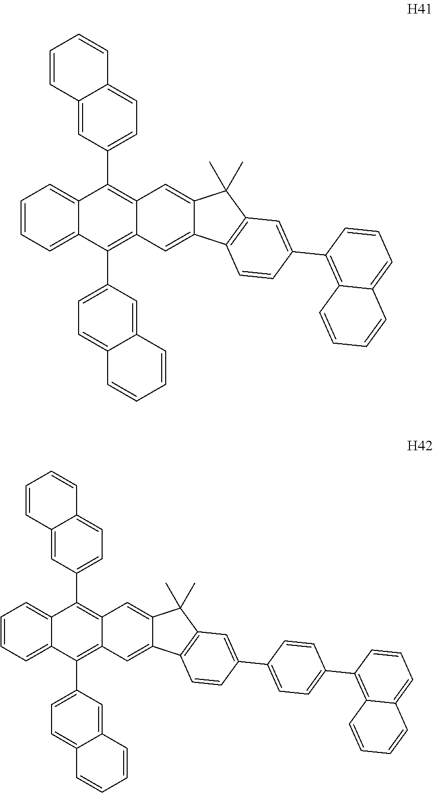

In some embodiments, the host may include at least one selected from Compounds H43 to H49 below, but the host is not limited thereto:

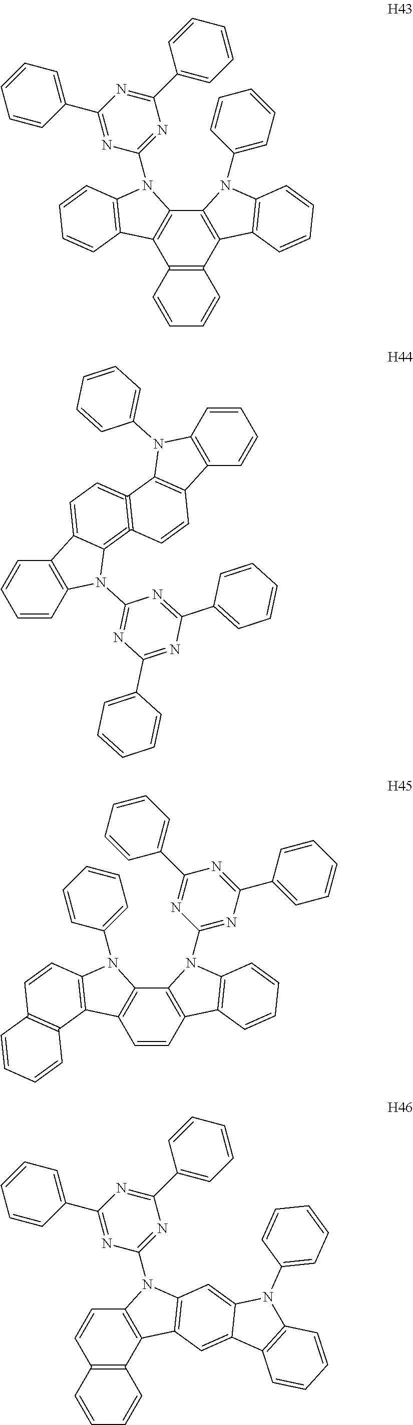

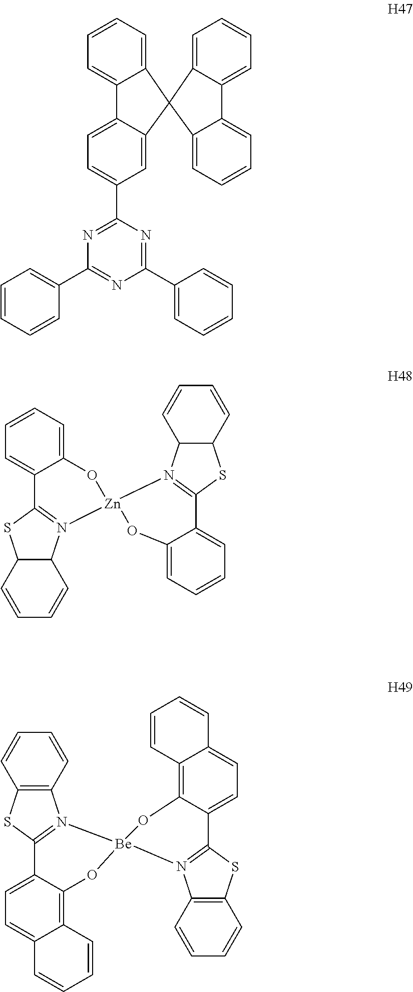

The dopant may include the compound represented by Formula 1 according to an example embodiment.

An amount of the dopant included in the emission layer may be, in general, in a range of about 0.01 to about 15 parts by weight based on 100 parts by weight of the host, but the dopant is not limited thereto.

A thickness of the emission layer may be in a range of about 100 Å to about 1,000 Å, for example, about 200 Å to about 600 Å. When the thickness of the emission layer is within the ranges described above, excellent light-emission characteristics are obtained without a substantial increase in driving voltage.

Next, the electron transport region may be disposed on the emission layer.

The electron transport region may include at least one selected from an HBL, an ETL, and an EIL, but the electron transport region is not limited thereto.

When the electron transport region includes an HBL, the HBL may be formed on the emission layer by utilizing one or more suitable methods, such as vacuum deposition, spin coating, casting, an LB method, an ink-jet printing, a laser-printing, or an LITI method. When the HBL is formed by vacuum deposition and/or spin coating, the deposition and coating conditions for the HBL may be determined by referring to the deposition and coating conditions for the HIL.

The HBL may include, for example, at least one selected from BCP and Bphen below, but the HBL is not limited thereto:

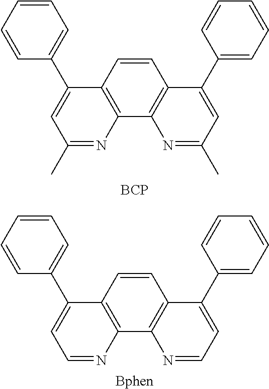

A thickness of the HBL may be in a range of about 20 Å to about 1,000 Å, for example, about 30 Å to about 300 Å. When the thickness of the HBL is within the ranges described above, excellent hole blocking characteristics are obtained without a substantial increase in driving voltage.

The electron transport region may have a structure of ETL/EIL or a structure of HBL/ETL/EIL. In the foregoing structures, layers of each structure are sequentially stacked from the emission layer, but the electron transport region is not limited thereto.

According to an example embodiment, the organic layer 150 of the organic light-emitting device 10 may include the electron transport region between the emission layer and the second electrode 190, and the electron transport region may include the ETL. The ETL may include a plurality of layers. For example, the electron transport region may include a first electron transport layer and a second electron transport layer.

The ETL may include at least one selected from BCP, Bphen, Alq₃, Balq, TAZ, and NTAZ below:

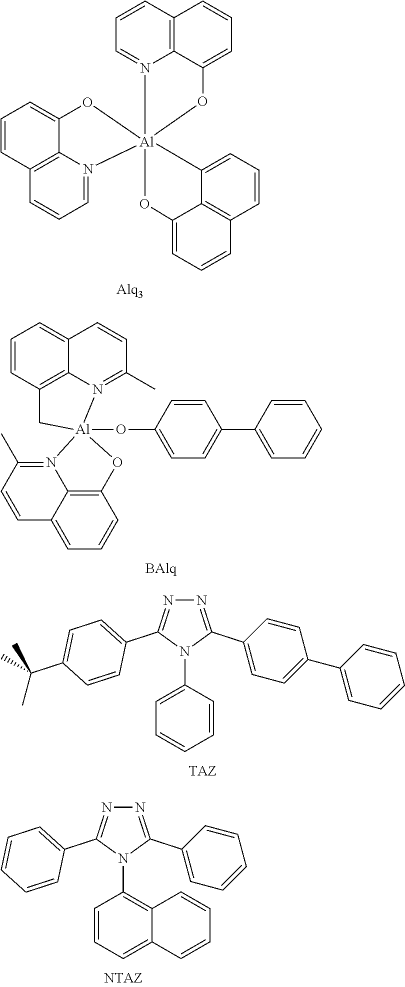

In some embodiments, the ETL may include at least one selected from a compound represented by Formula 601 and a compound represented by Formula 602:

Ar₆₀₁-[(L₆₀₁)ₓₑ₁-E₆₀₁]ₓₑ₂ Formula 601

In Formula 601,

Ar₆₀₁ may be selected from:

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, naphthacene, a picene, a perylene, a pentaphene, and an indenoanthracene;

a naphthalene, a heptalene, a fluorene, a spiro-fluorene, a benzofluorene, a dibenzofluorene, a phenalene, a phenanthrene, an anthracene, a fluoranthene, a triphenylene, a pyrene, a chrysene, naphthacene, a picene, a perylene, a pentaphene, and an indenoanthracene, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ heterocycloalkyl group, a C₃-C₁₀ cycloalkenyl group, a C₃-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group and —Si(Q₃₀₁)(Q₃₀₂)(Q₃₀₃) (wherein Q₃₀₁ to Q₃₀₃ may each independently be selected from a hydrogen, a C₁-C₆₀ alkyl group, a C₂-C₆₀ alkenyl group, a C₆-C₆₀ aryl group, and a C₂-C₆₀ heteroaryl group),

L₆₀₁ may be understood by referring to the descriptions provided herein in connection with L₂₀₃,

E₆₀₁ may be selected from:

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group; and

a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from a deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group,

xe1 may be selected from 0, 1, 2, and 3, and

xe2 may be selected from 1, 2, 3, and 4.

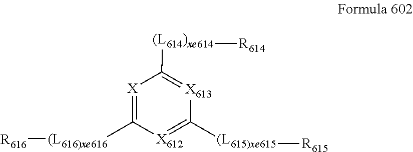

In Formula 602,

X₆₁₁ may be N or C-(L₆₁₁)ₓₑ₆₁₁-R₆₁₁, X₆₁₂ may be N or C-(L₆₁₂)ₓₑ₆₁₂-R₆₁₂, and X₆₁₃ may be N or C-(L₆₁₃)ₓₑ₆₁₃-R₆₁₃, wherein at least one of X₆₁₁ to X₆₁₃ is nitrogen (N),

L₆₁₁ to L₆₁₆ may be understood by referring to the descriptions provided herein in connection with L₂₀₃,

R₆₁₁ to R₆₁₆ may each be independently selected from:

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group; and

a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a fluorenyl group, a spiro-fluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazolyl group, and a triazinyl group, and

xe611 to xe616 may each be independently selected from 0, 1, 2, and 3.

The compound represented by Formula 601 and the compound represented by Formula 602 may each include at least one selected from Compounds ET1 to ET15 below:

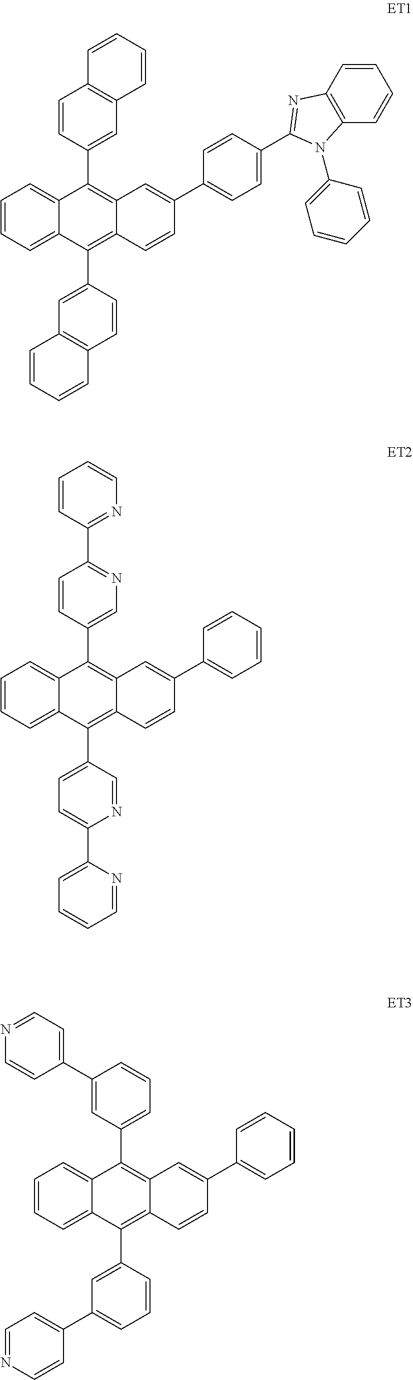 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

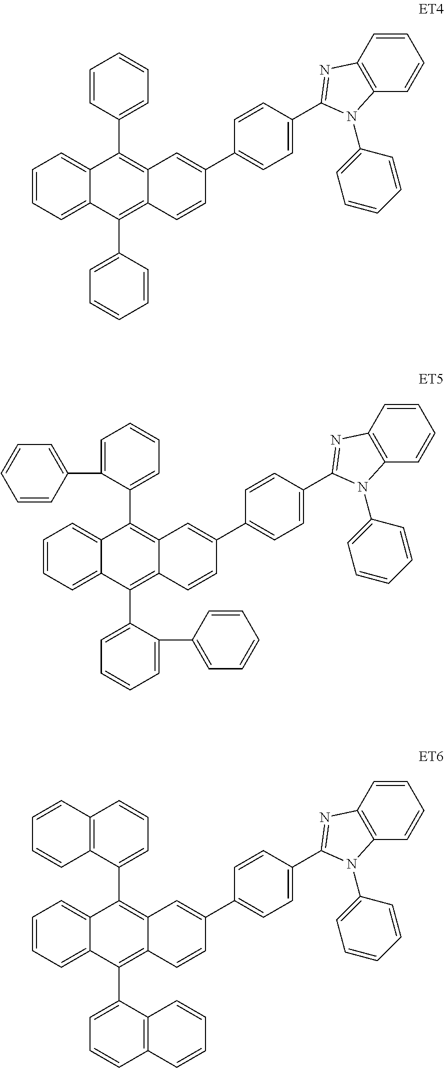 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

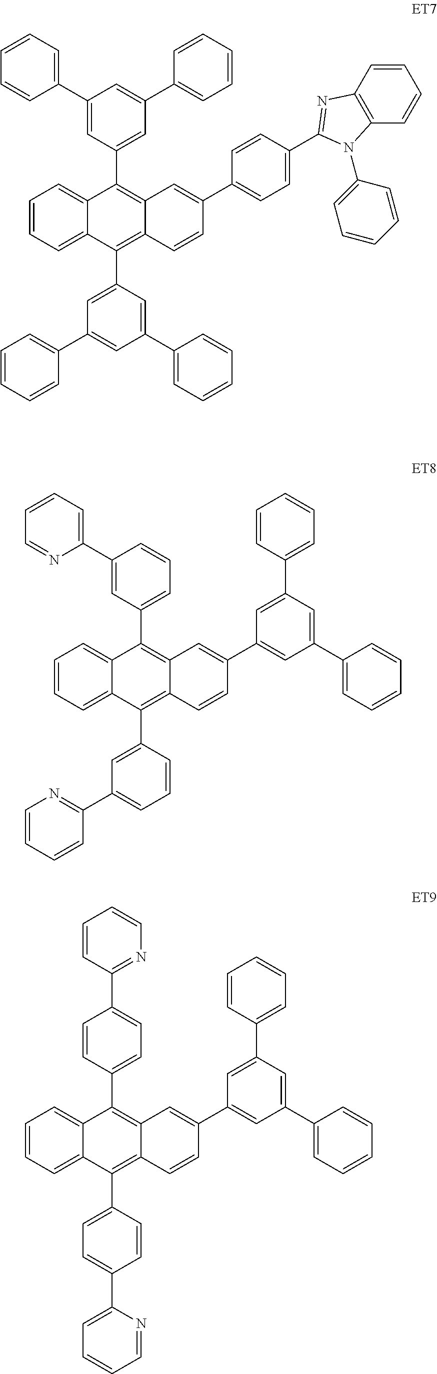 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

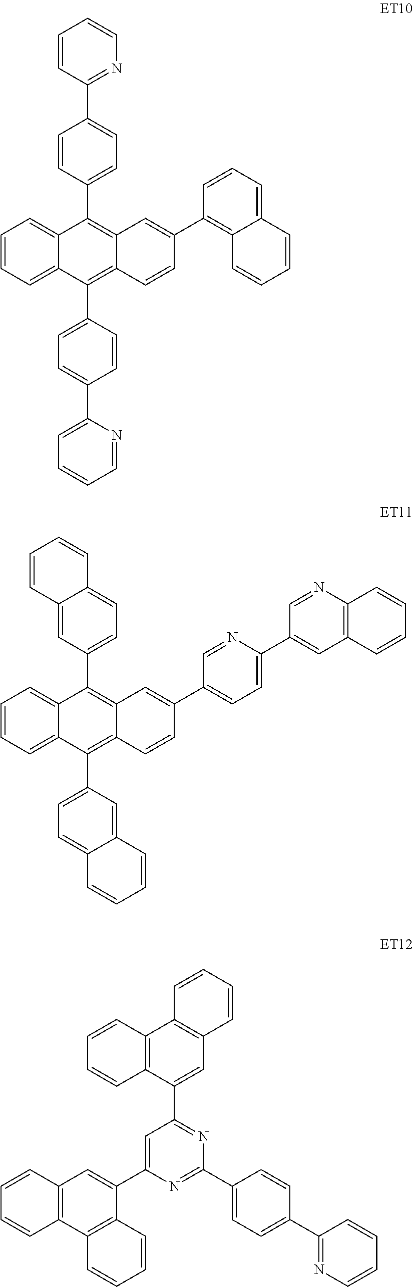 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-c2ccc(-c3nc(-c4cc5ccccc5c5ccccc45)cc(-c4cc5ccccc5c5ccccc45)n3)cc2)nc1]

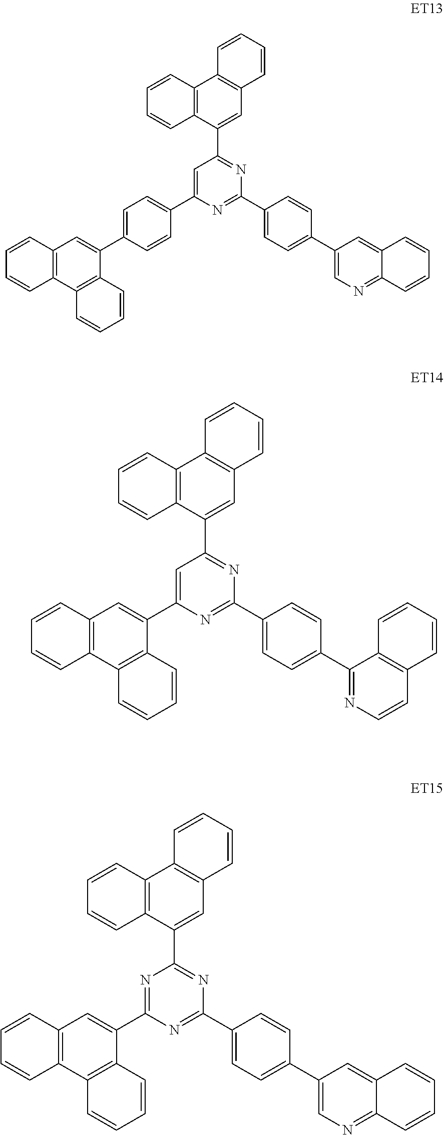 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

A thickness of the ETL may be in a range of about 100 Å to about 1,000 Å, for example, about 150 Å to about 500 Å. When the thickness of the ETL is within the ranges described above, the ETL has satisfactory or suitable electron transport characteristics without a substantial increase in driving voltage.

The ETL may further include, in addition to the materials described above, a metal-containing material.

The metal-containing material may include a Li complex. The Li complex may include, for example, Compound ET-D1 (lithium quinolate, LiQ) or ET-D2 below:

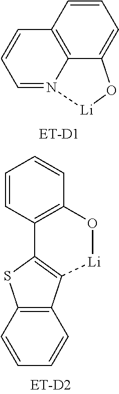

The electron transport region may include an EIL that facilitates electron injection from the second electrode 190.

The EIL may be formed on the ETL by utilizing one or more suitable methods, such as vacuum deposition, spin coating, casting, an LB method, an ink-jet printing, a laser-printing, or an LITI method. When the EIL is formed by vacuum deposition and/or spin coating, the deposition and coating conditions for the EIL may be determined by referring to the deposition and coating conditions for the HIL.

The EIL may include at least one selected from LiF, NaCl, CsF, Li₂O, BaO, and LiQ.

A thickness of the EIL may be in a range of about 1 Å to about 100 Å, for example, about 3 Å to about 90 Å. When the thickness of the EIL is within the ranges described above, the EIL has satisfactory or suitable electron injection characteristics without a substantial increase in driving voltage.

The second electrode 190 is disposed on the organic layer 150. The second electrode 190 may be a cathode that is an electron injection electrode. Here, a material for forming the second electrode 190 may include a metal, an alloy, an electrically conductive compound, or a mixture thereof, which has a relatively low work function. Examples of material for forming the second electrode 190 include lithium (Li), magnesium (Mg), aluminum (Al), aluminum-lithium (Al—Li), calcium (Ca), and magnesium-indium (Mg—In), magnesium-silver (Mg—Ag). In some embodiments, the material for forming the second electrode 190 may be ITO or IZO. The second electrode 190 may be a reflective electrode, a semi-transparent electrode, or a transparent electrode.

The organic layer 150 of the organic light-emitting device 10 may be formed by a deposition method using compounds according to an example embodiment, or by a wet coating method using compounds that are prepared in solutions according to an example embodiment.

The organic light-emitting device 10 according to an example embodiment may be included in various suitable types of flat panel display apparatus, such as a passive matrix OLED display apparatus and an active matrix OLED display apparatus. For example, when the organic light-emitting device 10 is equipped with the active matrix OLED display apparatus, the first electrode 110 disposed on a side of the substrate may serve as a pixel electrode, and may be electrically coupled to source and drain electrodes of a thin film transistor. In some embodiments, the organic light-emitting device 10 may be equipped with a flat panel display apparatus that can have display screens at both sides.

Hereinbefore, the organic light-emitting device 10 has been described with reference to the accompanying drawing, but the organic light-emitting device is not limited thereto.

Hereinafter, representative substituents among all of the substituents used in the present disclosure may be defined as follows (carbon numbers limiting the substituents are non-limiting and do not limit characteristics of the substituents, and substituents that are not described in the present disclosure are not included if found in general definitions of the substituents, e.g., substituents that are not described herein should have the same meaning as commonly understood by one of ordinary skill in the art to which the present disclosure pertains).

The term “C₁-C₆₀ alkyl group,” as used herein, refers to a linear or branched aliphatic hydrocarbon monovalent group having 1 to 60 carbon atoms, and examples thereof include a methyl group, an ethyl group, a propyl group, an isobutyl group, a sec-butyl group, a ter-butyl group, a pentyl group, an iso-amyl group, and a hexyl group. The term “C₁-C₆₀ alkylene group,” as used herein, refers to a divalent group having substantially the same structure as the C₁-C₆₀ alkyl group except that the C₁-C₆₀ alkylene group is divalent instead of monovalent.

The term “C₁-C₆₀ alkoxy group,” as used herein, refers to a monovalent group represented by —OA₁₀₁ (wherein A₁₀₁ is the same as the C₁-C₆₀ alkyl group), and examples thereof include a methoxy group, an ethoxy group, and an isopropyloxy group.

The term “C₂-C₆₀ alkenyl group,” as used herein, refers to a hydrocarbon group formed by substituting at least one carbon-carbon double bond in a main chain (e.g., in the middle of the chain) or at a terminal end of the C₂-C₆₀ alkyl group, and examples thereof include an ethenyl group, a propenyl group, and a butenyl group. The term “C₂-C₆₀ alkenylene group,” as used herein, refers to a divalent group having substantially the same structure as the C₂-C₆₀ alkenyl group except that the C₂-C₆₀ alkenylene group is divalent instead of monovalent.

The term “C₂-C₆₀ alkynyl group,” as used herein, refers to a hydrocarbon group formed by substituting at least one carbon-carbon triple bond in a main chain (e.g., in the middle of the chain) or at a terminal end of the C₂-C₆₀ alkyl group, and examples thereof include an ethynyl group and a propynyl group. The term “C₂-C₆₀ alkynylene group,” as used herein, refers to a divalent group having substantially the same structure as the C₂-C₆₀ alkynyl group except that the C₂-C₆₀ alkynylene group is divalent instead of monovalent.

The term “C₃-C₁₀ cycloalkyl group,” as used herein, refers to a monovalent hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group,” as used herein, refers to a divalent group having substantially the same structure as the C₃-C₁₀ cycloalkyl group except that the C₃-C₁₀ cycloalkylene group is divalent instead of monovalent.

The term “C₁-C₁₀ heterocycloalkyl group,” as used herein, refers to a monovalent monocyclic group having at least one heteroatom selected from N, O, P, and S as a ring-forming atom and 1 to 10 carbon atoms, and examples thereof include a tetrahydrofuranyl group and a tetrahydrothiophenyl group. The term “C₂-C₁₀ heterocycloalkylene group,” as used herein, refers to a divalent group having substantially the same structure as the C₂-C₁₀ heterocycloalkyl group except that the C₂-C₁₀ heterocycloalkylene group is divalent instead of monovalent.

The term “C₃-C₁₀ cycloalkenyl group,” as used herein, refers to a monovalent monocyclic group that has 3 to 10 carbon atoms and at least one double bond (e.g., at least one carbon-carbon double bond) in a ring thereof and does not have aromaticity (e.g., the ring or the C₃-C₁₀ cycloalkenyl group is not aromatic), and examples thereof include a cyclopentenyl group, a cyclohexenyl group, and a cycloheptenyl group. The term “C₃-C₁₀ cycloalkenylene group,” as used herein, refers to a divalent group having substantially the same structure as the C₃-C₁₀ cycloalkenyl group except that the C₃-C₁₀ cycloalkenylene group is divalent instead of monovalent.

The term “C₂-C₁₀ heterocycloalkenyl group,” as used herein, refers to a monovalent monocyclic group that has at least one heteroatom selected from N, O, P, and S as a ring-forming atom, 2 to 10 carbon atoms, and at least one double bond (e.g., at least one carbon-carbon double bond) in its ring. Examples of the C₂-C₁₀ heterocycloalkenyl group include a 2,3-hydrofuranyl group and a 2,3-hydrothiophenyl group. The term “C₂-C₁₀ heterocycloalkenylene group,” as used herein, refers to a divalent group having substantially the same structure as the C₂-C₁₀ heterocycloalkenyl group except that the C₂-C₁₀ heterocycloalkenylene group is divalent instead of monovalent.

The term “C₆-C₆₀ aryl group,” as used herein, refers to a monovalent group having a carbocyclic aromatic system having 6 to 60 carbon atoms, and the term “C₆-C₆₀ arylene group,” as used herein, refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, these rings may be fused to each other (e.g., combined together).

The term “C₁-C₆₀ heteroaryl group,” as used herein, refers to a monovalent group having a carbocyclic aromatic system that has at least one heteroatom selected from N, O, P, and S as a ring-forming atom, and 1 to 60 carbon atoms. The term “C₁-C₆₀ heteroarylene group,” as used herein, refers to a divalent group having a carbocyclic aromatic system that has at least one heteroatom selected from N, O, P, and S as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, these rings may be fused to each other (e.g., combined together).

The term “C₆-C₆₀ aryloxy group,” as used herein, indicates —OA₁₀₂ (wherein A₁₀₂ is the C₆-C₆₀ aryl group), and the term “C₆-C₆₀ arylthio group,” as used herein, indicates —SA₁₀₃ (wherein A₁₀₃ is the C₆-C₆₀ aryl group).

The term “monovalent non-aromatic condensed polycyclic group” (e.g., a group having 8 to 60 carbon atoms), as used herein, refers to a monovalent group that has two or more rings condensed to each other (e.g., combined together), has only carbon atoms only as ring-forming atoms, and has non-aromaticity in the entire molecular structure (e.g., the entire monovalent non-aromatic condensed polycyclic group is not aromatic, although the group may be bonded to another group that is aromatic). An example of the monovalent non-aromatic condensed polycyclic group is a fluorenyl group. The term “divalent non-aromatic condensed polycyclic group,” as used herein, refers to a divalent group having substantially the same structure as the monovalent non-aromatic condensed polycyclic group except that the divalent non-aromatic condensed polycyclic group is divalent instead of monovalent.

The term “monovalent non-aromatic condensed heteropolycyclic group” (e.g., a group having 2 to 60 carbon atoms), as used herein, refers to a monovalent group that has two or more rings condensed to each other (e.g., combined together), has heteroatoms as a ring-forming atom selected from N, O, P, and S, in addition to C, and has non-aromaticity in the entire molecular structure (e.g., the entire monovalent non-aromatic condensed polycyclic group is not aromatic, although the group may be bonded to another group that is aromatic). An example of the monovalent non-aromatic condensed heteropolycyclic group is a carbazolyl group. The term “divalent non-aromatic condensed heteropolycyclic group,” as used herein, refers to a divalent group having the same structure as the monovalent non-aromatic condensed heteropolycyclic group except that the divalent non-aromatic condensed heteropolycyclic group is divalent instead of monovalent.

At least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₆), and —B(Q₁₆)(Q₁₇);

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅) and —B(Q₃₆)(Q₃₇),

wherein Q₁₁ to Q₁₇, Q₂₁ to Q₂₇, and Q₃₁ to Q₃₇ may each be independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

For example, at least one substituent of the substituted C₃-C₁₀ cycloalkylene group, the substituted C₂-C₁₀ heterocycloalkylene group, the substituted C₃-C₁₀ cycloalkenylene group, the substituted C₂-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, a ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —N(Q₁₁)(Q₁₂), —Si(Q₁₃)(Q₁₄)(Q₁₅), and —B(Q₁₆)(Q₁₇);

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, a ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group;

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, a ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group, each substituted with at least one selected from a deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, a ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, an imidazopyrimidinyl group, —N(Q₂₁)(Q₂₂), —Si(Q₂₃)(Q₂₄)(Q₂₅), and —B(Q₂₆)(Q₂₇); and

—N(Q₃₁)(Q₃₂), —Si(Q₃₃)(Q₃₄)(Q₃₅), and —B(Q₃₆)(Q₃₇),

wherein Q₁₁ to Q₁₇, Q₂₁ to Q₂₇, and Q₃₁ to Q₃₇ may each be independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-fluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, a ovalenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, a thiadiazolyl group, an imidazopyridinyl group, and an imidazopyrimidinyl group.

The term “Ph,” as used herein, refers to a phenyl group, the term “Me,” as used herein, refers to a methyl group, the term “Et,” as used herein, refers to an ethyl group, and the term “ter-Bu” or “Bu^{t},” as used herein, refers to a tert-butyl group.

Hereinafter, an organic light-emitting device according to an embodiment will be described in more detail with reference to Synthesis Examples and Examples.

### Synthesis Example

### Synthesis Example 1

### Synthesis of Intermediate A1

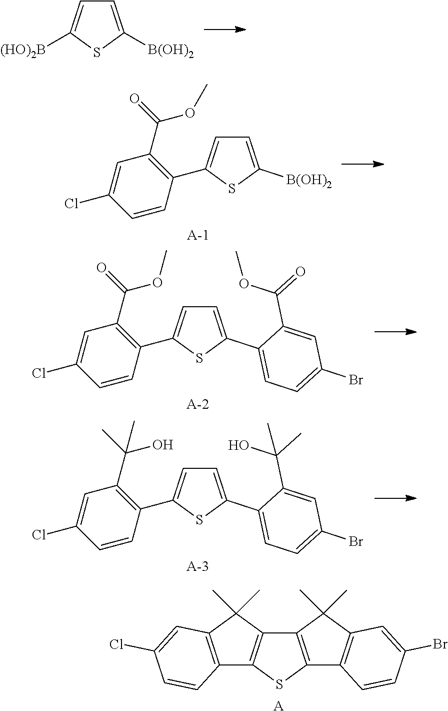

### Synthesis of Intermediate A-1

In a nitrogen atmosphere, 5.13 g (30 mmol) of thiophene-2,5-diyldiboronic acid, 7.472 g (30 mmol) of methyl 2-bromo-5-chlorobenzoate, 1.732 g (1.5 mmol) of Pd(PPh₃)₄, and 6.21 g (45 mmol) of K₂CO₃ were dissolved in 500 mL of a mixed solution of THF/H₂O (at a volume ratio of 2/1), and then, stirred at a temperature of 80° C. for 12 hours. After the reaction solution was cooled to room temperature, 100 mL of water was added thereto and the resulting solution was extracted 3 times with 150 mL of ethylether. An organic solvent layer collected therefrom was dried with magnesium sulfate, and then, the residues obtained by evaporating the solvent were separated-purified by silica gel chromatography, so as to obtain 6.21 g (21 mmol, yield of: 83%) of Intermediate A-1.

### Synthesis of Intermediate A-2

6.97 g (15 mmol, yield of: 71%) of Intermediate A-2 was obtained in the same manner as in Synthesis of Intermediate A-1, except that Intermediate A-1 and methyl 5-bromo-2-iodobenzoate were used instead of thiophene-2,5-diyldiboronic acid and methyl 2-bromo-5-chlorobenzoate, respectively.

### Synthesis of Intermediate A-3

In a nitrogen atmosphere, 6.97 g (15 mmol) of Intermediate A-1 was dissolved in 500 ml of anhydrous THF, and then, stirred at a temperature of 0° C. for 1 hour. 30 mL of 1.6 M methylmagnesium bromide hexane solution was slowly added dropwise thereto for 1 hour, and then, stirred at room temperature for 24 hours. Continuously, 50 mL of 1N HCl was added thereto, and the resulting solution was extracted 3 times with 150 mL of ethylether. An organic solvent layer collected therefrom was dried with magnesium sulfate, and then, the residues obtained by evaporating the solvent were separated-purified by silica gel chromatography, so as to obtain 6.51 g (14 mmol, yield of: 93%) of Intermediate A-3.

### Synthesis of Intermediate A

In a nitrogen atmosphere, 6.51 g (14 mmol) of Intermediate A-3 was dissolved in 100 mL of dichloromethane, and then, stirred at a temperature of 0° C. for 1 hour. 5 mL of methane sulfonic acid was slowly added dropwise thereto for 30 minutes. After the reaction solution was stirred at room temperature for 1 hour, 50 mL of sodium carbonate aqueous solution was added thereto and the resulting solution was extracted 3 times with 50 mL of dichloromethane. An organic solvent layer collected therefrom was dried with magnesium sulfate, and then, the residues obtained by evaporating the solvent were separated-purified by silica gel chromatography, so as to obtain 4.29 g (10 mmol, yield of: 71%) of Intermediate A.

### Synthesis Example 2

### Synthesis of Intermediate B

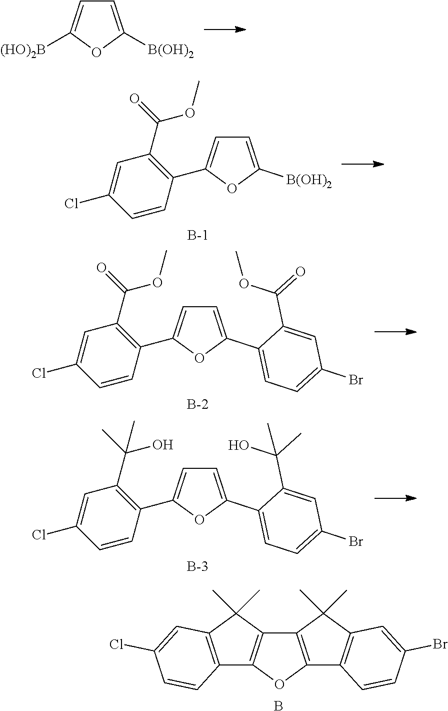

### Synthesis of Intermediate B-1

6.16 g (22 mmol, yield of: 73%) of Intermediate B-1 was obtained in the same manner as described with respect to Synthesis of Intermediate A-1, except that furan-2,5-diyldiboronic acid was used instead of thiophene-2,5-diyldiboronic acid.

### Synthesis of Intermediate B-2

7.18 g (16 mmol, yield of: 72%) of Intermediate B-2 was obtained in the same manner as described with respect to Synthesis of Intermediate A-2, except that Intermediate B-1 was used instead of Intermediate A-1.

### Synthesis of Intermediate B-3

6.26 g (13 mmol, yield of: 81%) of Intermediate B-3 was obtained in the same manner as described with respect to Synthesis of Intermediate A-3, except that Intermediate B-2 was used instead of Intermediate A-2.

### Synthesis of Intermediate B

4.54 g (11 mmol, yield of: 84%) of Intermediate B was obtained in the same manner as described with respect to Synthesis of Intermediate A, except that Intermediate B-3 was used instead of Intermediate A-2.

### Synthesis Example 3

### Synthesis of Compound 1

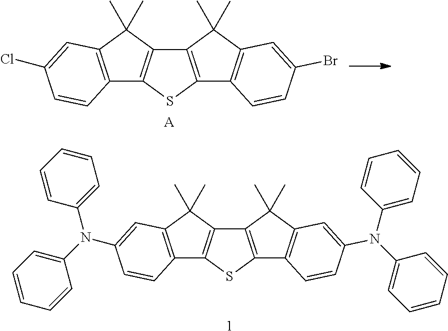

In a nitrogen atmosphere, 0.429 g (1 mmol) of Intermediate A, 0.507 g (3 mmol) of di-phenylamine, 0.091 g (0.1 mmol) of tris(dibenzylideneacetone)dipalladium(0) (Pd₂(dba)₃), 0.020 g (0.1 mmol) of tri-tert-butylphosphine (P(t-Bu)₃), and 0.288 g (3 mmol) of NaOtBu were dissolved in 60 mL of toluene, and then, stirred at a temperature of 90° C. for 4 hours. After the reaction solution was cooled to room temperature, the resulting solution was extracted 3 times, each with 50 ml of water and 50 mL of di ethylether. An organic layer collected therefrom was dried with magnesium sulfate, and then, the residues obtained by evaporating the solvent were separated-purified by silica gel chromatography, so as to obtain 0.520 g (0.8 mmol, yield of: 80%) of Compound 1.

### Synthesis Example 4

### Synthesis of Compound 39

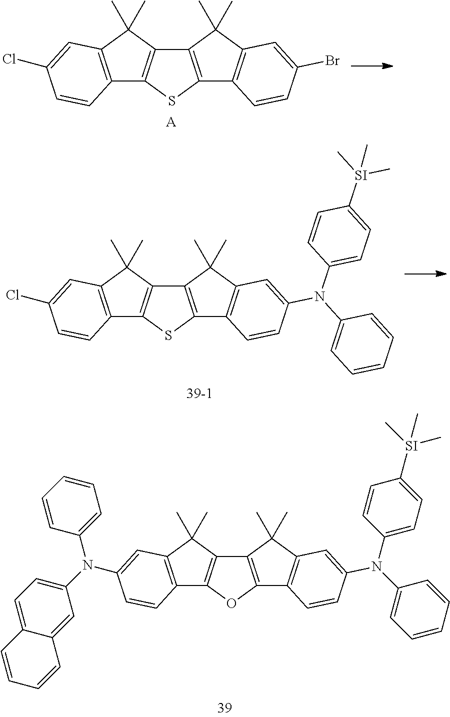

### Synthesis of Intermediate 39-1

0.500 g (0.87 mmol, yield: 87%) of Intermediate 39-1 was obtained in the same manner as described with respect to Synthesis of Compound 1, except that N-phenyl-4-(trimethylsilyl)aniline was used instead of di-phenylamine.

### Synthesis of Compound 39

0.540 g (0.7 mmol, yield: 80%) of Compound 39 was obtained in the same manner as described with respect to Synthesis of Compound 1, except that Intermediate 39-1 and N-phenylnaphthalen-2-amine were used instead of Intermediate A and di-phenylamine, respectively.

Other additional compounds were synthesized according to the same synthetic routes and the same synthesis methods as those described above, except that, in the additional syntheses, intermediate materials appropriate for each synthesis were utilized. In addition to the compounds described in the present specification, other compounds may be also easily synthesized by one of ordinary skill in the art, in view of the present specification, by referring to the synthetic routes and raw materials described above.

### Example 1

As an anode substrate, a 15 Ω/cm² (1,200 Å) ITO glass substrate (manufactured by Corning) was cut into a size of 50 mm×50 mm×0.7 mm and ultrasonically washed out with isopropyl alcohol and pure water, each for 5 minutes. The ITO glass substrate was irradiated by ultraviolet light (UV) for 30 minutes, cleaned by exposing to ozone, and then, transported to a vacuum evaporator.

2-TNATA was vacuum deposited on the ITO anode to form an HIL having a thickness of 600 Å, and 4,4′-bis[N-(1-naphthyl)-N-phenylan amino group]biphenyl (NPB) was deposited on the HIL to form an HTL having a thickness of 300 Å.

Then, 9,10-di-naphthalene-2-yl-anthracene (DNA) below and Compound 1 were co-deposited at a weight ratio of 98:2 on the HTL to form an emission layer having a thickness of 300 Å.

Then, Alq₃ was deposited on the emission layer to form an ETL having a thickness of 300 Å, and LiF was deposited on the ETL to form an EIL having a thickness of 10 Å. Al was deposited on the EIL to form a second electrode (i.e., a cathode) having a thickness of 3,000 Å, thereby manufacturing an organic light-emitting device.

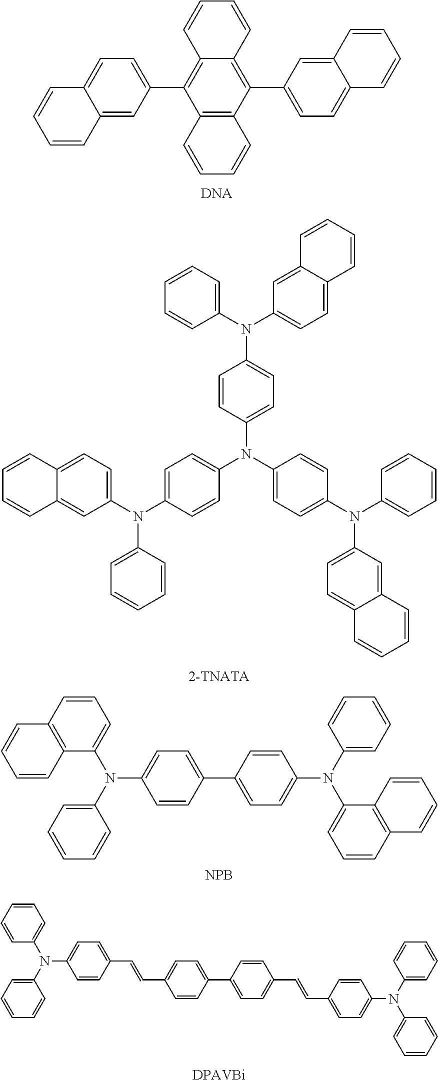

### Example 2

An organic light-emitting device was manufactured in the same manner as described with respect to Example 1, except that in forming the EML, Compound 20 was used instead of Compound 1.

### Example 3

An organic light-emitting device was manufactured in the same manner as described with respect to Example 1, except that in forming the EML, Compound 39 was used instead of Compound 1.

### Example 4

An organic light-emitting device was manufactured in the same manner as described with respect to Example 1, except that in forming the EML, Compound 49 was used instead of Compound 1.

### Example 5

An organic light-emitting device was manufactured in the same manner as described with respect to Example 1, except that in forming the EML, Compound 70 was used instead of Compound 1.

### Example 6

An organic light-emitting device was manufactured in the same manner as described with respect to Example 1, except that in forming the EML, Compound 93 was used instead of Compound 1.

### Example 7

An organic light-emitting device was manufactured in the same manner as described with respect to Example 1, except that in forming the EML, Compound 98 was used instead of Compound 1.

### Example 8

An organic light-emitting device was manufactured in the same manner as described with respect to Example 1, except that in forming the EML, Compound 125 was used instead of Compound 1.

### Comparative Example 1

An organic light-emitting device was manufactured in the same manner as described with respect to Example 1, except that in forming the emission layer, DPAVBi, which has been utilized as a blue fluorescent dopant in the art, was used instead of Compound 1.

The characteristics of the organic light-emitting devices prepared in Examples 1 to 8 and Comparative Example 1 are shown in Table 1 below.

TABLE 1

| | | Driving | Current | | | | |
|---|---|---|---|---|---|---|---|
| | | voltage | density | Brightness | Efficiency | Emitting | Half-lifespan |
| | Material | (V) | (mA/cm²) | (cd/m²) | (cd/A) | color | (hr @100 mA/cm²) |
| Example 1 | Compound 1 | 5.96 | 50 | 3005 | 6.01 | blue | 315 |
| Example 2 | Compound 20 | 6.02 | 50 | 2995 | 5.99 | blue | 320 |
| Example 3 | Compound 39 | 5.99 | 50 | 3108 | 6.21 | blue | 305 |
| Example 4 | Compound 49 | 6.24 | 50 | 3069 | 6.14 | blue | 335 |
| Example 5 | Compound 70 | 5.89 | 50 | 3052 | 6.10 | blue | 300 |
| Example 6 | Compound 93 | 5.90 | 50 | 3041 | 6.08 | blue | 290 |
| Example 7 | Compound 98 | 6.01 | 50 | 2900 | 5.80 | blue | 308 |
| Example 8 | Compound125 | 6.10 | 50 | 2950 | 5.90 | blue | 315 |
| Comparative | DPAVBi | 7.01 | 50 | 2645 | 5.29 | blue | 258 |
| Example 1 | | | | | | | |

Referring to Table 1, it was confirmed that when the compound of Formula 1 was used as a dopant for forming the emission layer, the driving voltage of the light-emitting device including the compound represented by Formula 1 was lower than that of the light-emitting device of Comparative Example 1. The light-emitting device including the compound represented by Formula 1 also exhibited significantly increased efficiency and I-V-L characteristics, and in particular, showed excellent lifespan characteristics.

As described above, an organic light-emitting device including a compound according to one or more of the above-described embodiments may have good emission characteristics, and thus may be suitable for fluorescent and/or phosphorescent devices of all colors including red, green, blue, and white. Accordingly, an organic light-emitting device having high efficiency, low driving voltage, high brightness, and long lifespan characteristics may be manufactured.

It will be understood that when an element or layer is referred to as being “on,” “connected to,” or “coupled to” another element or layer, it can be directly on, connected to, or coupled to the other element or layer, or one or more intervening elements or layers may be present. For example, in the context of the present disclosure, the emission layer may be directly or indirectly on the hole transport region. In addition, it will also be understood that when an element or layer is referred to as being “between” two elements or layers, it can be the only element or layer between the two elements or layers, or one or more intervening elements or layers may also be present.

It should be understood that example embodiments described herein should be considered in a descriptive sense only and not for purposes of limitation. Descriptions of features or aspects within each example embodiment should typically be considered as available for other similar features or aspects in other example embodiments.

While one or more example embodiments have been described with reference to the figures, it will be understood by those of ordinary skill in the art that various changes in form and details may be made therein without departing from the spirit and scope of the present disclosure as defined by the following claims, and equivalents thereof.

What is claimed is:

1. A compound represented by Formula 1: 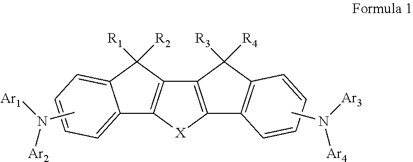
wherein:
R₁ to R₄ are each independently selected from:
a hydrogen, a deuterium, —F, —Cl, —Br, —I, a hydroxyl group, a cyano group, a nitro group, an amino group, an am idino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,
Ar₁ to Ar₄ are each independently selected from a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group;
X is selected from oxygen (O), sulfur (S), and selenium (Se), and
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₂-C₆₀ heteroaryl group, the substituted monovalent non-aromatic condensed polycyclic group, and the substituted monovalent non-aromatic condensed heteropolycyclic group is selected from:
a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), and —B(Q₁₆)(Q₁₇);
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycyclic group;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, and a monovalent non-aromatic condensed heteropolycyclic group, each substituted with at least one selected from a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₂-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), and —B(Q₂₆)(Q₂₇); and
—Si(Q₃₁)(Q₃₂)(Q₃₃),
wherein Q₁₁ to Q₁₇, Q₂₁ to Q₂₇, and Q₃₁ to Q₃₃ are each independently selected from a hydrogen, a deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

2. The compound of claim 1, wherein in Formula 1, R₁ to R₄ are each independently a substituted or unsubstituted C₁-C₆₀ alkyl group.

3. The compound of claim 1, wherein in Formula 1, Ar₁ to Ar₄ are each independently selected from groups represented by Formulae 2a to 2d: 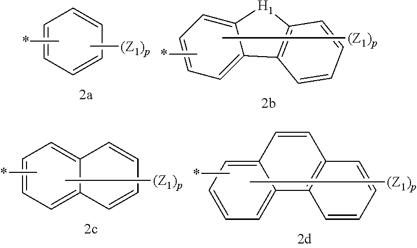
wherein in Formulae 2a to 2d,
H₁ is selected from CR₁₁R₁₂, O, and S,
R₁₁, R₁₂, and Z₁ are each independently selected from a hydrogen, a deuterium, a halogen group, a cyano group, a nitro group, a hydroxyl group, a carboxyl group, a substituted or unsubstituted C₁-C₂₀ alkyl group, a substituted or unsubstituted C₆-C₂₀ aryl group, a substituted or unsubstituted C₁-C₂₀ heteroaryl group, a substituted or unsubstituted monovalent non-aromatic condensed polycyclic group, a substituted or unsubstituted monovalent non-aromatic condensed heteropolycyclic group, and —Si(Q₃₁)(Q₃₂)(Q₃₃),
wherein when the compound comprises a plurality of Z₁s, each of the Z₁s is identical to or different from the others of the Z₁s,
p is an integer selected from 1 to 9, and
* indicates a binding site.

4. The compound of claim 1, wherein in Formula 1, X is O or S.

5. The compound of claim 1, wherein the compound of Formula 1 is represented by Formula 2:

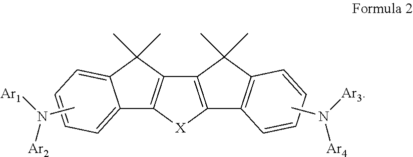

6. The compound of claim 1, wherein the compound of Formula 1 is represented by Formula 3:

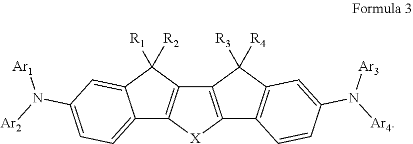

7. The compound of claim 1, wherein the compound of Formula 1 is represented by Formula 4:

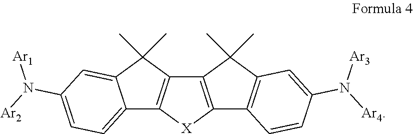

8. The compound of claim 1, wherein the compound represented by Formula 1 is one of the following compounds:

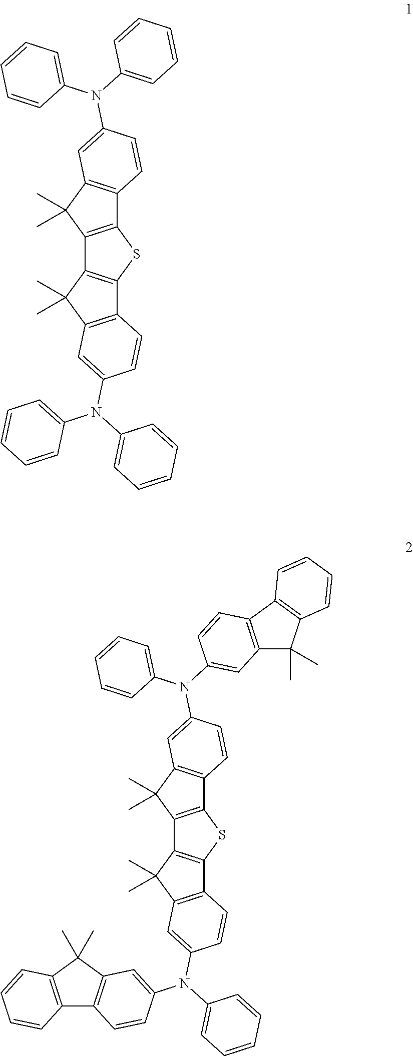 [SMILES: CC1(C)c2cc(N(c3ccccc3)c3ccccc3)ccc2-c2sc3c(c21)C(C)(C)c1cc(N(c2ccccc2)c2ccccc2)ccc1-3 ; CC1(C)c2ccccc2-c2ccc(N(c3ccccc3)c3ccc4c(c3)C(C)(C)c3c-4sc4c3C(C)(C)c3cc(N(c5ccccc5)c5ccc6c(c5)C(C)(C)c5ccccc5-6)ccc3-4)cc21]

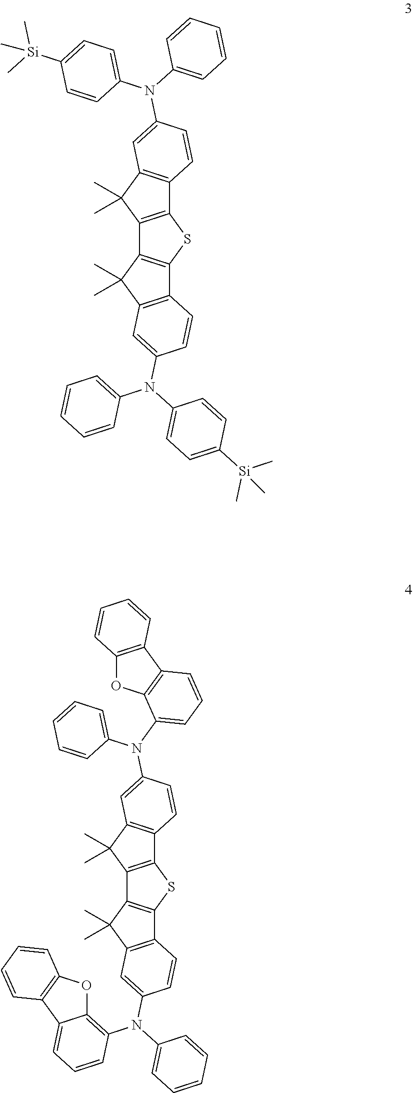

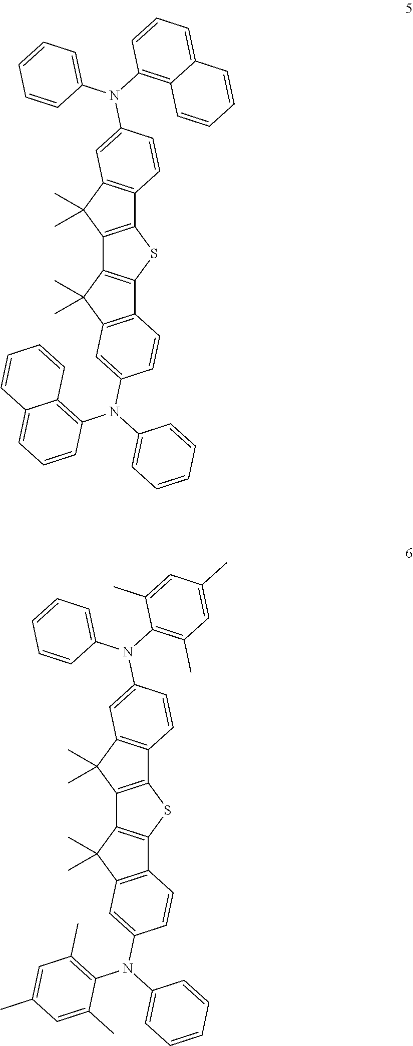

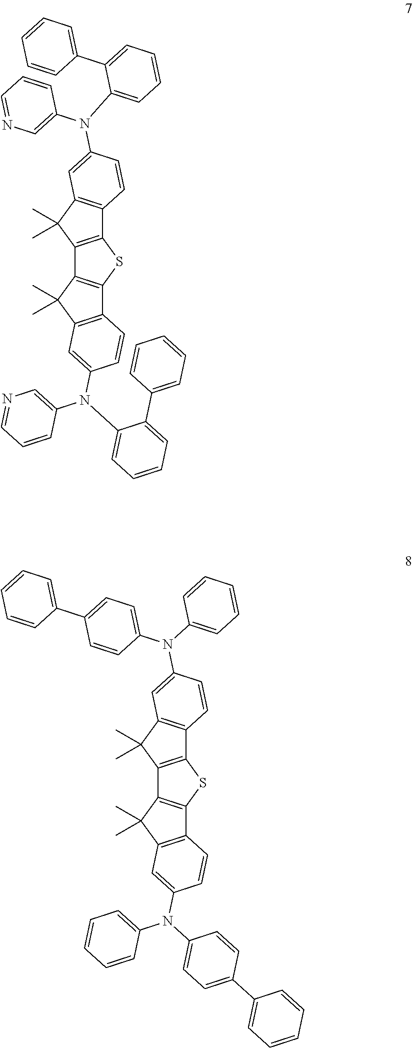 [SMILES: CC1(C)c2cc(N(c3cccnc3)c3ccccc3-c3ccccc3)ccc2-c2sc3c(c21)C(C)(C)c1cc(N(c2cccnc2)c2ccccc2-c2ccccc2)ccc1-3 ; CC1(C)c2cc(N(c3ccccc3)c3ccc(-c4ccccc4)cc3)ccc2-c2sc3c(c21)C(C)(C)c1cc(N(c2ccccc2)c2ccc(-c4ccccc4)cc2)ccc1-3]

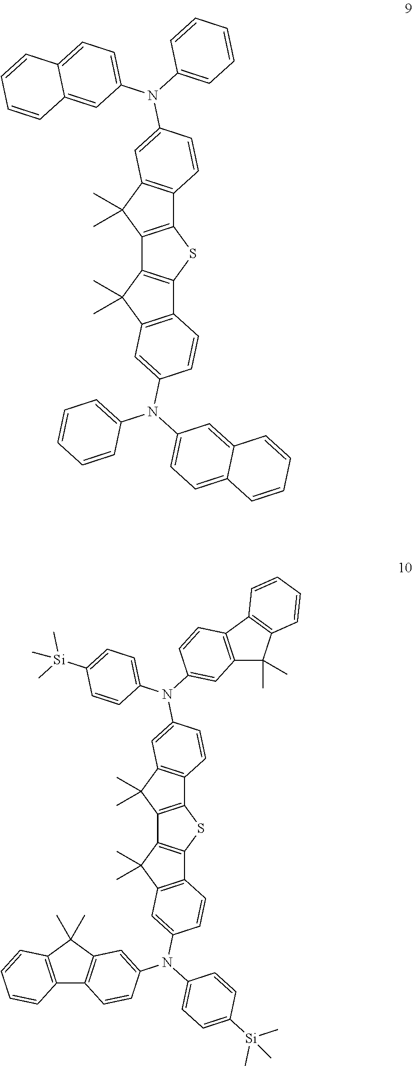 [SMILES: CC1(C)c2cc(N(c3ccccc3)c3ccc4ccccc4c3)ccc2-c2sc3c(c21)C(C)(C)c1cc(N(c2ccccc2)c2ccc4ccccc4c2)ccc1-3 ; CC1(C)c2ccccc2-c2ccc(N(c3ccc([Si](C)(C)C)cc3)c3ccc4c(c3)C(C)(C)c3c-4sc4c3C(C)(C)c3cc(N(c5ccc([Si](C)(C)C)cc5)c5ccc6c(c5)C(C)(C)c5ccccc5-6)ccc3-4)cc21]

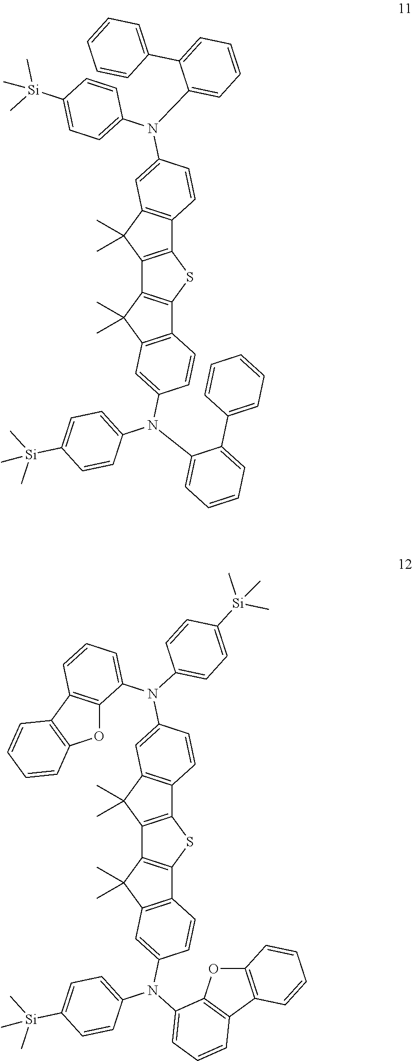 [SMILES: CC1(C)c2cc(N(c3ccc([Si](C)(C)C)cc3)c3ccccc3-c3ccccc3)ccc2-c2sc3c(c21)C(C)(C)c1cc(N(c2ccc([Si](C)(C)C)cc2)c2ccccc2-c2ccccc2)ccc1-3 ; CC1(C)c2cc(N(c3ccc([Si](C)(C)C)cc3)c3cccc4c3oc3ccccc34)ccc2-c2sc3c(c21)C(C)(C)c1cc(N(c2ccc([Si](C)(C)C)cc2)c2cccc4c2oc2ccccc24)ccc1-3]

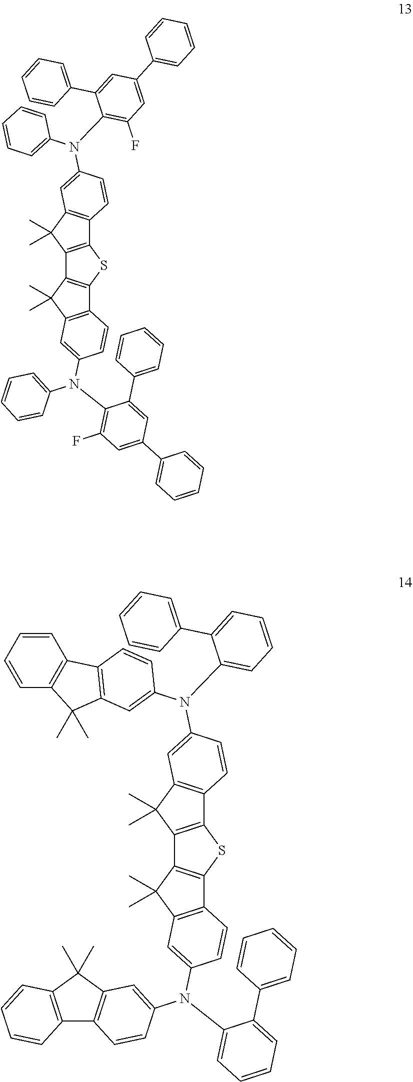 [SMILES: CC1(C)c2cc(N(c3ccccc3)c3c(F)cc(-c4ccccc4)cc3-c3ccccc3)ccc2-c2sc3c(c21)C(C)(C)c1cc(N(c2ccccc2)c2c(F)cc(-c4ccccc4)cc2-c2ccccc2)ccc1-3 ; CC1(C)c2ccccc2-c2ccc(N(c3ccc4c(c3)C(C)(C)c3c-4sc4c3C(C)(C)c3cc(N(c5ccc6c(c5)C(C)(C)c5ccccc5-6)c5ccccc5-c5ccccc5)ccc3-4)c3ccccc3-c3ccccc3)cc21]

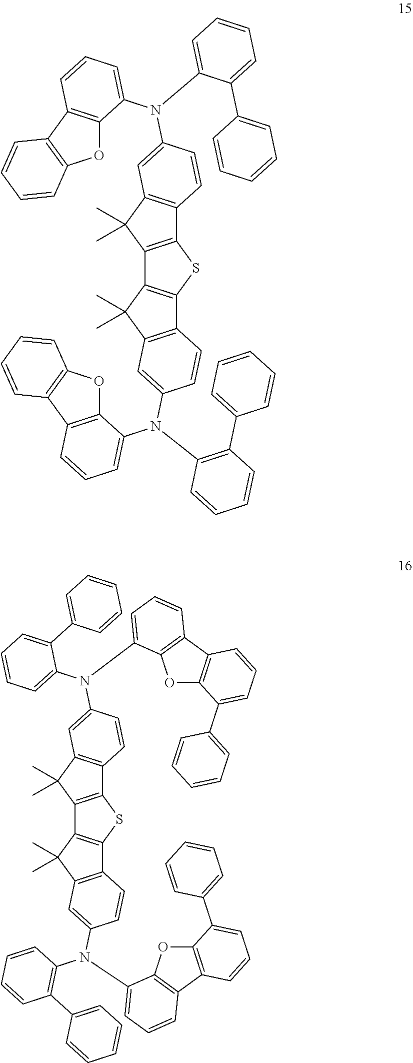 [SMILES: CC1(C)c2cc(N(c3ccccc3-c3ccccc3)c3cccc4c3oc3ccccc34)ccc2-c2sc3c(c21)C(C)(C)c1cc(N(c2ccccc2-c2ccccc2)c2cccc4c2oc2ccccc24)ccc1-3 ; CC1(C)c2cc(N(c3ccccc3-c3ccccc3)c3cccc4c3oc3c(-c5ccccc5)cccc34)ccc2-c2sc3c(c21)C(C)(C)c1cc(N(c2ccccc2-c2ccccc2)c2cccc4c2oc2c(-c5ccccc5)cccc24)ccc1-3]

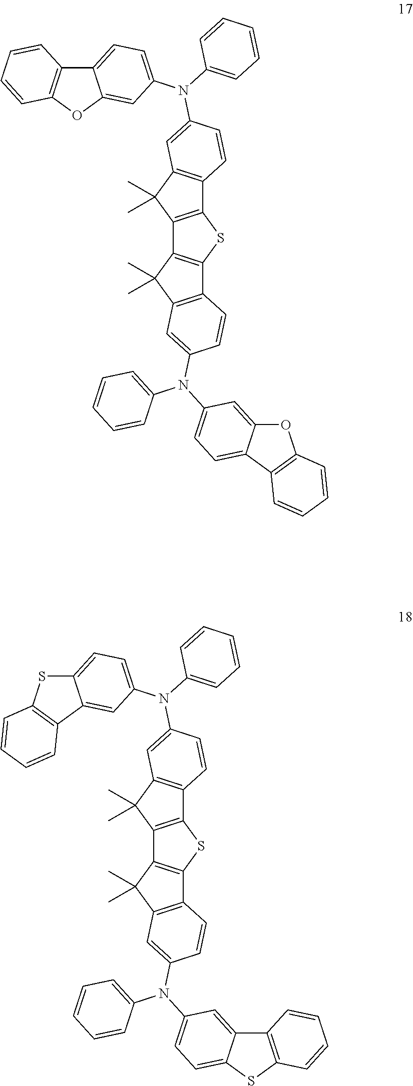 [SMILES: CC1(C)c2cc(N(c3ccccc3)c3ccc4c(c3)oc3ccccc34)ccc2-c2sc3c(c21)C(C)(C)c1cc(N(c2ccccc2)c2ccc4c(c2)oc2ccccc24)ccc1-3 ; CC1(C)c2cc(N(c3ccccc3)c3ccc4sc5ccccc5c4c3)ccc2-c2sc3c(c21)C(C)(C)c1cc(N(c2ccccc2)c2ccc4sc5ccccc5c4c2)ccc1-3]

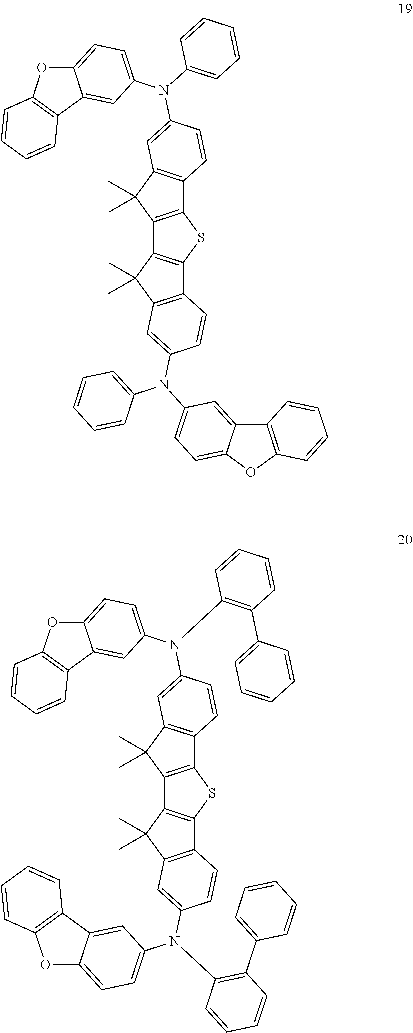 [SMILES: CC1(C)c2cc(N(c3ccccc3)c3ccc4oc5ccccc5c4c3)ccc2-c2sc3c(c21)C(C)(C)c1cc(N(c2ccccc2)c2ccc4oc5ccccc5c4c2)ccc1-3 ; CC1(C)c2cc(N(c3ccc4oc5ccccc5c4c3)c3ccccc3-c3ccccc3)ccc2-c2sc3c(c21)C(C)(C)c1cc(N(c2ccc4oc5ccccc5c4c2)c2ccccc2-c2ccccc2)ccc1-3]

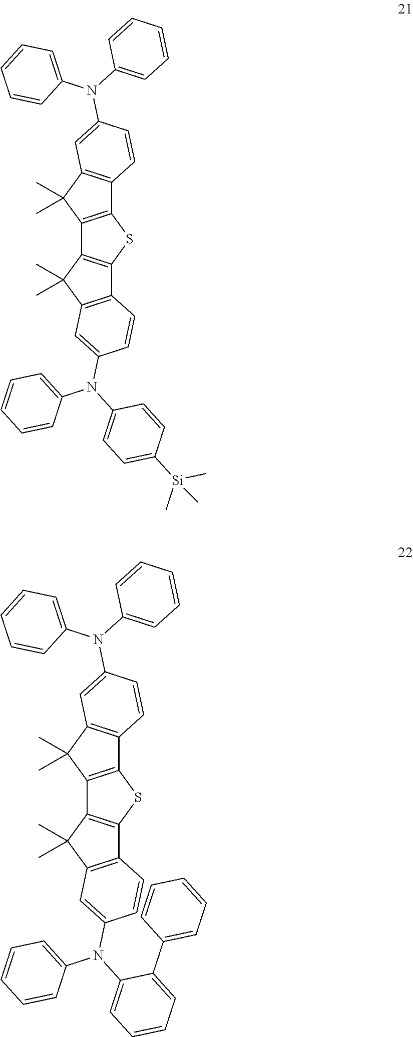 [SMILES: CC1(C)c2cc(N(c3ccccc3)c3ccccc3)ccc2-c2sc3c(c21)C(C)(C)c1cc(N(c2ccccc2)c2ccc([Si](C)(C)C)cc2)ccc1-3 ; CC1(C)c2cc(N(c3ccccc3)c3ccccc3)ccc2-c2sc3c(c21)C(C)(C)c1cc(N(c2ccccc2)c2ccccc2-c2ccccc2)ccc1-3]

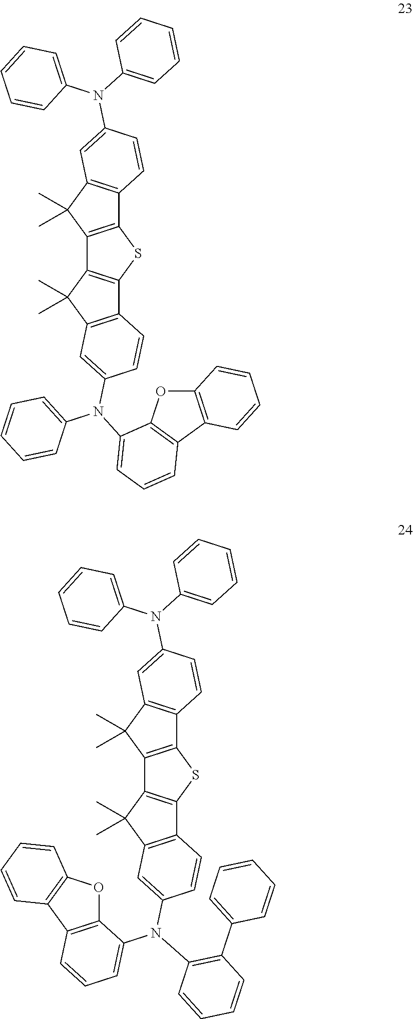 [SMILES: CC1(C)c2cc(N(c3ccccc3)c3ccccc3)ccc2-c2sc3c(c21)C(C)(C)c1cc(N(c2ccccc2)c2cccc4c2oc2ccccc24)ccc1-3 ; CC1(C)c2cc(N(c3ccccc3)c3ccccc3)ccc2-c2sc3c(c21)C(C)(C)c1cc(N(c2ccccc2-c2ccccc2)c2cccc4c2oc2ccccc24)ccc1-3]

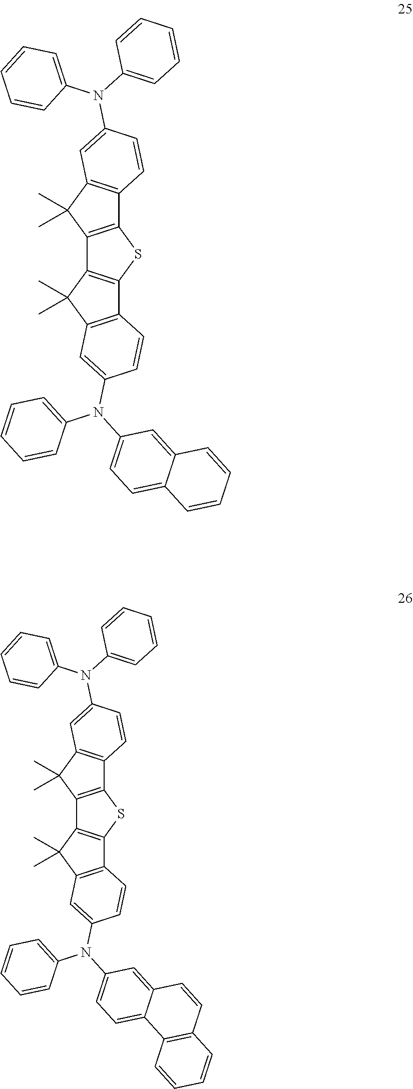 [SMILES: CC1(C)c2cc(N(c3ccccc3)c3ccccc3)ccc2-c2sc3c(c21)C(C)(C)c1cc(N(c2ccccc2)c2ccc4ccccc4c2)ccc1-3 ; CC1(C)c2cc(N(c3ccccc3)c3ccccc3)ccc2-c2sc3c(c21)C(C)(C)c1cc(N(c2ccccc2)c2ccc4c(ccc5ccccc54)c2)ccc1-3]

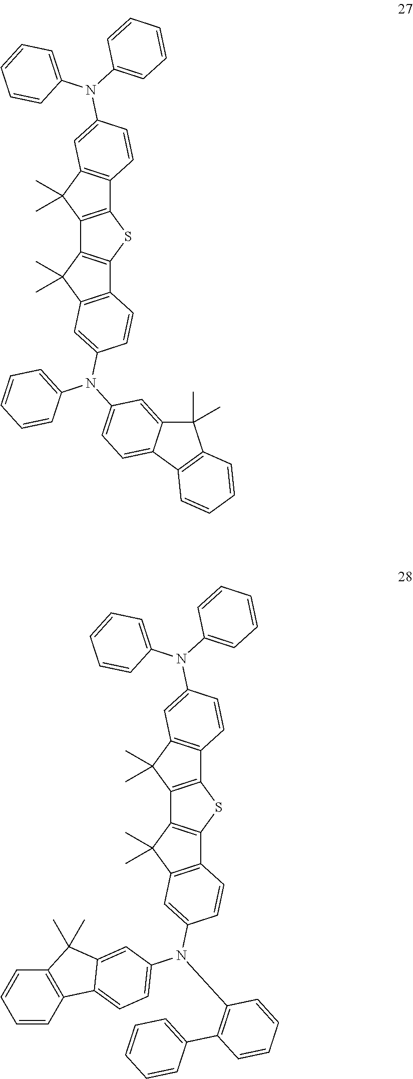 [SMILES: CC1(C)c2ccccc2-c2ccc(N(c3ccccc3)c3ccc4c(c3)C(C)(C)c3c-4sc4c3C(C)(C)c3cc(N(c5ccccc5)c5ccccc5)ccc3-4)cc21 ; CC1(C)c2ccccc2-c2ccc(N(c3ccc4c(c3)C(C)(C)c3c-4sc4c3C(C)(C)c3cc(N(c5ccccc5)c5ccccc5)ccc3-4)c3ccccc3-c3ccccc3)cc21]

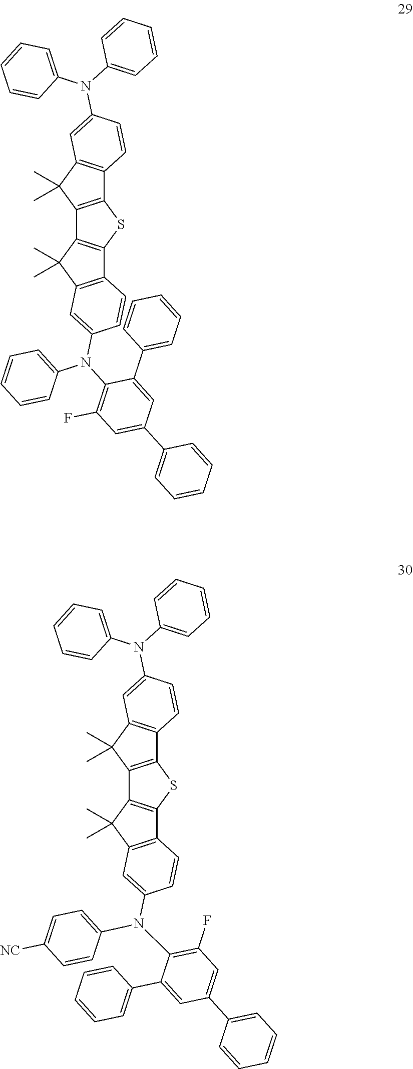 [SMILES: CC1(C)c2cc(N(c3ccccc3)c3ccccc3)ccc2-c2sc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccccc3)ccc2-c2sc3c(c21)C(C)(C)c1cc(N(c2ccc(C#N)cc2)c2c(F)cc(-c4ccccc4)cc2-c2ccccc2)ccc1-3]

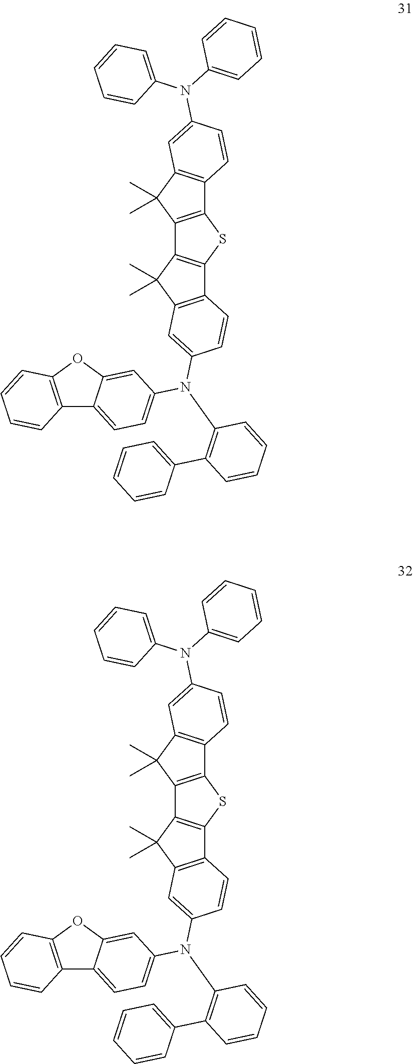 [SMILES: CC1(C)c2cc(N(c3ccccc3)c3ccccc3)ccc2-c2sc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccccc3)ccc2-c2sc3c(c21)C(C)(C)c1cc(N(c2ccc4c(c2)oc2ccccc24)c2ccccc2-c2ccccc2)ccc1-3]

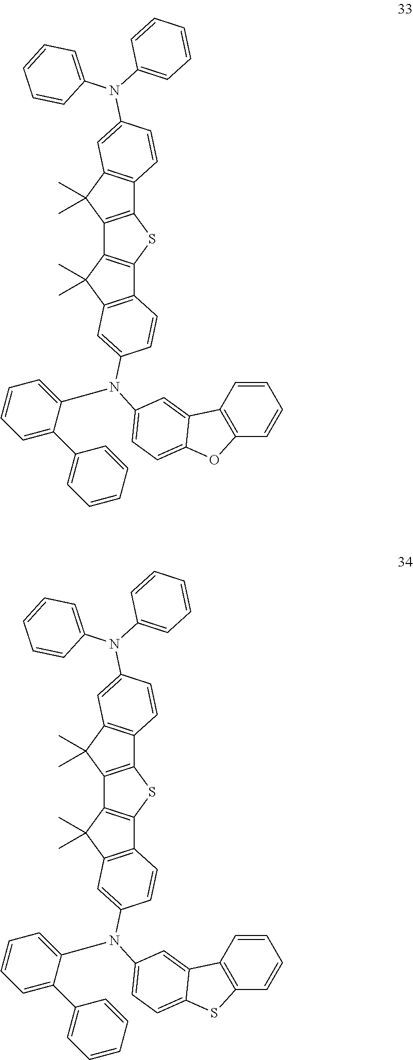 [SMILES: CC1(C)c2cc(N(c3ccccc3)c3ccccc3)ccc2-c2sc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2sc3c(c21)C(C)(C)c1cc(N(c2ccc4sc5ccccc5c4c2)c2ccccc2-c2ccccc2)ccc1-3]

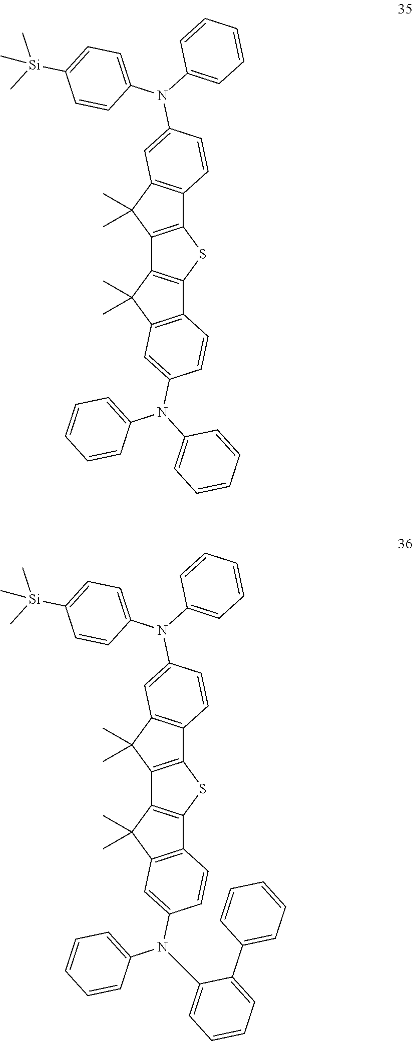 [SMILES: CC1(C)c2cc(N(c3ccccc3)c3ccccc3)ccc2-c2sc3c(c21)C(C)(C)c1cc(N(c2ccccc2)c2ccc([Si](C)(C)C)cc2)ccc1-3 ; CC1(C)c2cc(N(c3ccccc3)c3ccc([Si](C)(C)C)cc3)ccc2-c2sc3c(c21)C(C)(C)c1cc(N(c2ccccc2)c2ccccc2-c2ccccc2)ccc1-3]

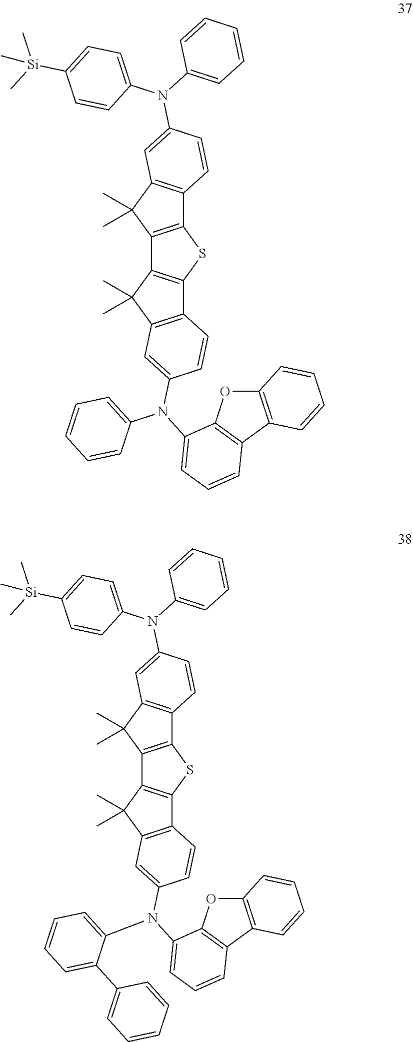 [SMILES: CC1(C)c2cc(N(c3ccccc3)c3ccc([Si](C)(C)C)cc3)ccc2-c2sc3c(c21)C(C)(C)c1cc(N(c2ccccc2)c2cccc4c2oc2ccccc24)ccc1-3 ; CC1(C)c2cc(N(c3ccccc3)c3ccc([Si](C)(C)C)cc3)ccc2-c2sc3c(c21)C(C)(C)c1cc(N(c2ccccc2-c2ccccc2)c2cccc4c2oc2ccccc24)ccc1-3]

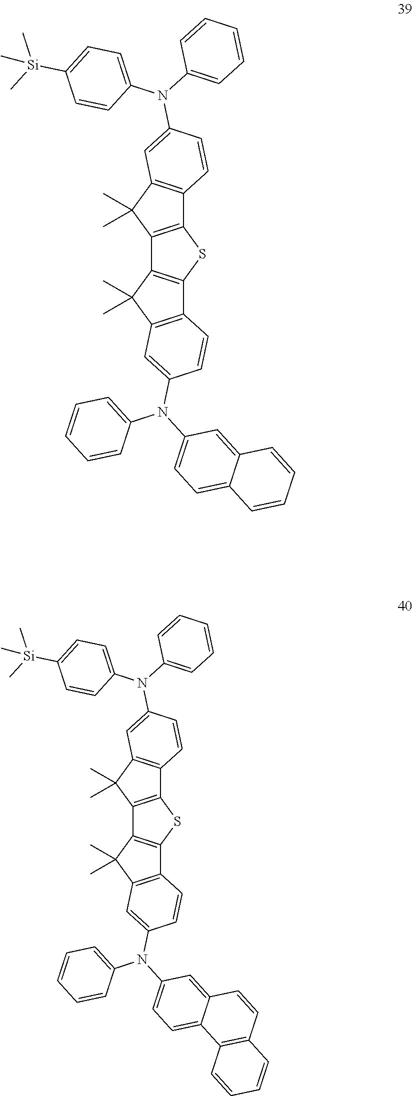 [SMILES: CC1(C)c2cc(N(c3ccccc3)c3ccc([Si](C)(C)C)cc3)ccc2-c2sc3c(c21)C(C)(C)c1cc(N(c2ccccc2)c2ccc4ccccc4c2)ccc1-3 ; CC1(C)c2cc(N(c3ccccc3)c3ccc([Si](C)(C)C)cc3)ccc2-c2sc3c(c21)C(C)(C)c1cc(N(c2ccccc2)c2ccc4c(ccc5ccccc54)c2)ccc1-3]

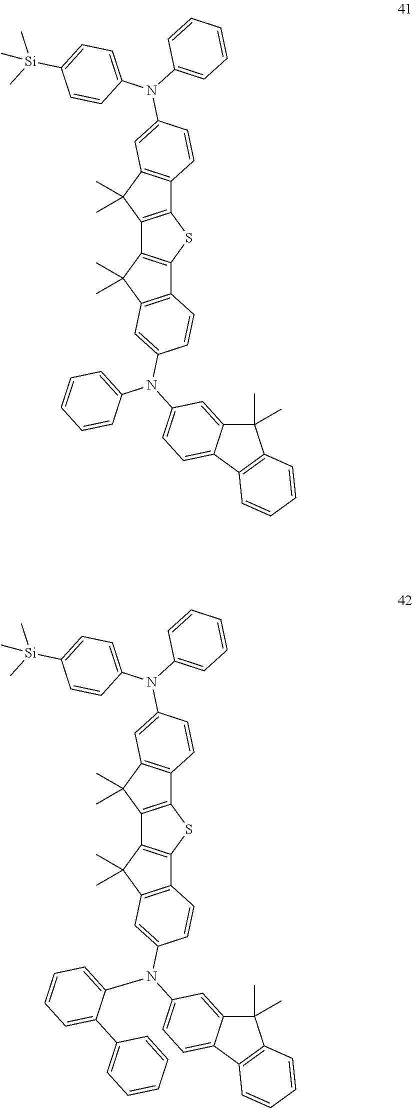 [SMILES: CC1(C)c2ccccc2-c2ccc(N(c3ccccc3)c3ccc4c(c3)C(C)(C)c3c-4sc4c3C(C)(C)c3cc(N(c5ccccc5)c5ccc([Si](C)(C)C)cc5)ccc3-4)cc21 ; CC1(C)c2ccccc2-c2ccc(N(c3ccc4c(c3)C(C)(C)c3c-4sc4c3C(C)(C)c3cc(N(c5ccccc5)c5ccc([Si](C)(C)C)cc5)ccc3-4)c3ccccc3-c3ccccc3)cc21]

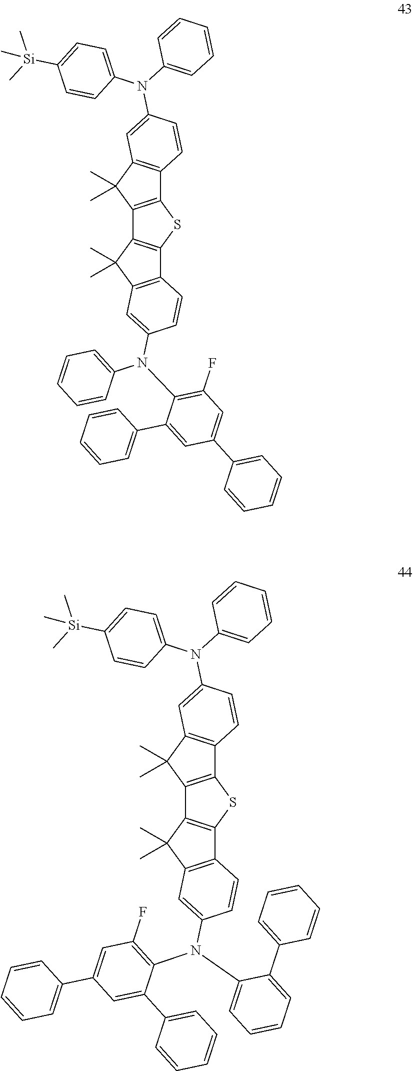 [SMILES: CC1(C)c2cc(N(c3ccccc3)c3ccc([Si](C)(C)C)cc3)ccc2-c2sc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccc([Si](C)(C)C)cc3)ccc2-c2sc3c(c21)C(C)(C)c1cc(N(c2ccccc2-c2ccccc2)c2c(F)cc(-c4ccccc4)cc2-c2ccccc2)ccc1-3]

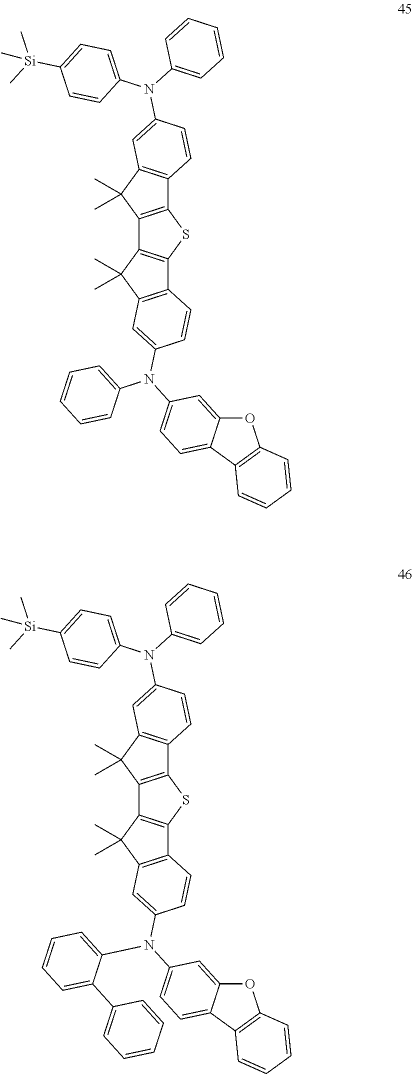 [SMILES: CC1(C)c2cc(N(c3ccccc3)c3ccc([Si](C)(C)C)cc3)ccc2-c2sc3c(c21)C(C)(C)c1cc(N(c2ccccc2)c2ccc4c(c2)oc2ccccc24)ccc1-3 ; CC1(C)c2cc(N(c3ccccc3)c3ccc([Si](C)(C)C)cc3)ccc2-c2sc3c(c21)C(C)(C)c1cc(N(c2ccc4c(c2)oc2ccccc24)c2ccccc2-c2ccccc2)ccc1-3]

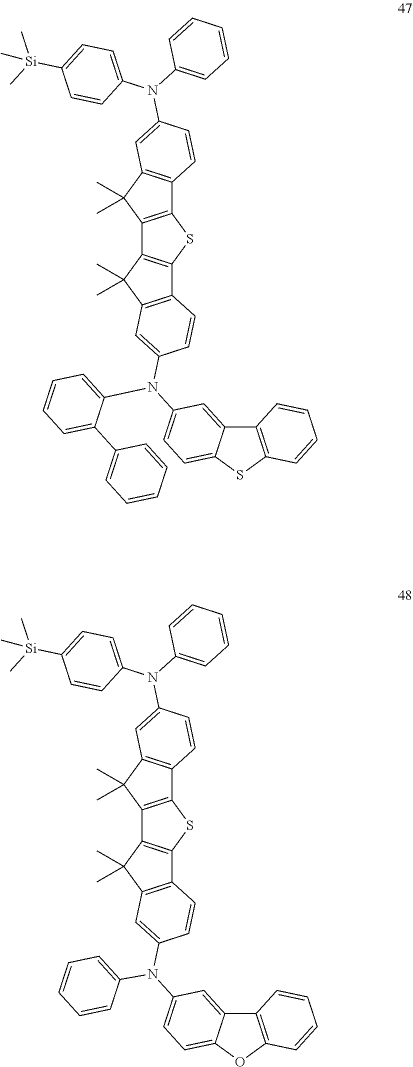 [SMILES: CC1(C)c2cc(N(c3ccccc3)c3ccc([Si](C)(C)C)cc3)ccc2-c2sc3c(c21)C(C)(C)c1cc(N(c2ccc4sc5ccccc5c4c2)c2ccccc2-c2ccccc2)ccc1-3 ; CC1(C)c2cc(N(c3ccccc3)c3ccc([Si](C)(C)C)cc3)ccc2-c2sc3c(c21)C(C)(C)c1cc(N(c2ccccc2)c2ccc4oc5ccccc5c4c2)ccc1-3]

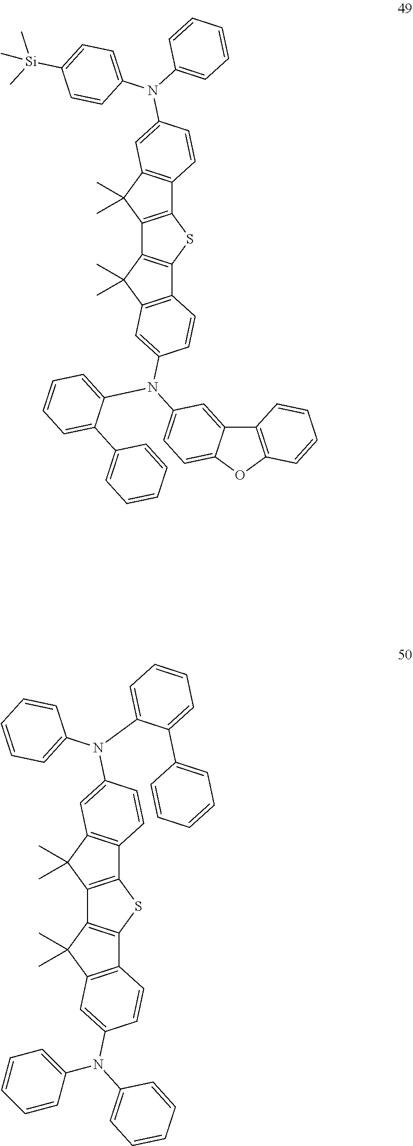 [SMILES: CC1(C)c2cc(N(c3ccccc3)c3ccc([Si](C)(C)C)cc3)ccc2-c2sc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2sc3c(c21)C(C)(C)c1cc(N(c2ccccc2)c2ccccc2-c2ccccc2)ccc1-3]

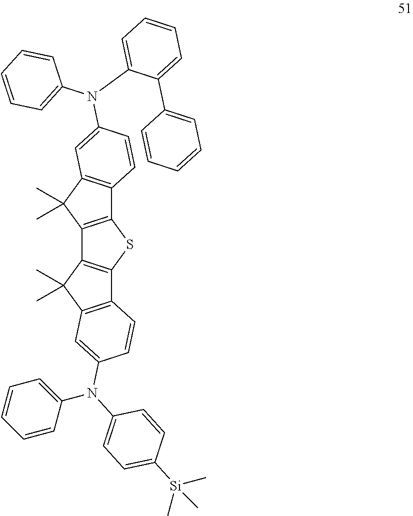 [SMILES: CC1(C)c2cc(N(c3ccccc3)c3ccc([Si](C)(C)C)cc3)ccc2-c2sc3c(c21)C(C)(C)c1cc(N(c2ccccc2)c2ccccc2-c2ccccc2)ccc1-3]

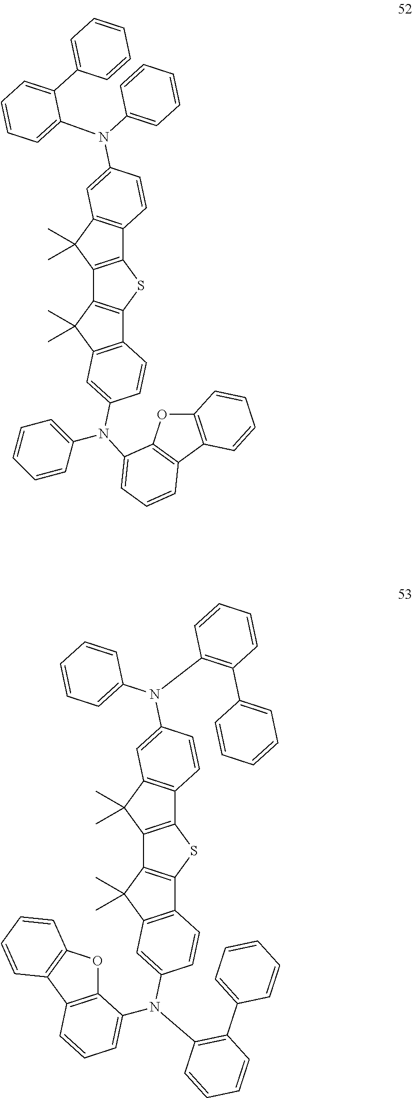 [SMILES: CC1(C)c2cc(N(c3ccccc3)c3ccccc3-c3ccccc3)ccc2-c2sc3c(c21)C(C)(C)c1cc(N(c2ccccc2)c2cccc4c2oc2ccccc24)ccc1-3 ; CC1(C)c2cc(N(c3ccccc3)c3ccccc3-c3ccccc3)ccc2-c2sc3c(c21)C(C)(C)c1cc(N(c2ccccc2-c2ccccc2)c2cccc4c2oc2ccccc24)ccc1-3]

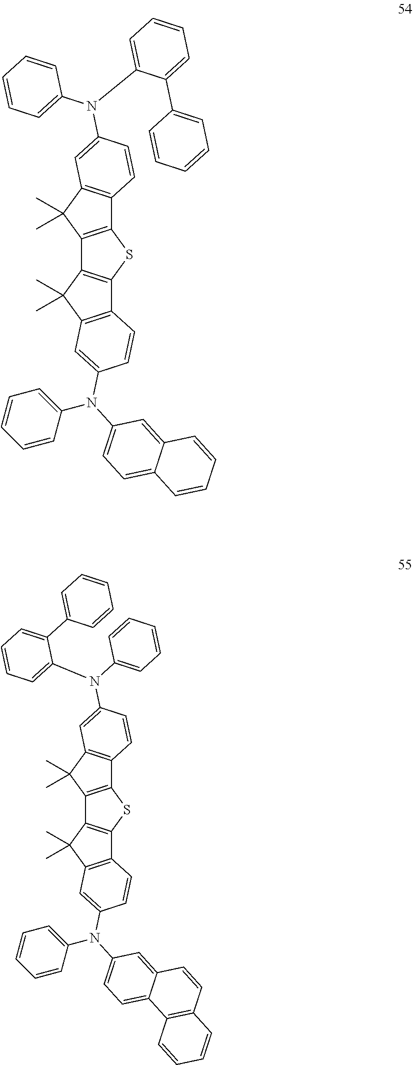 [SMILES: CC1(C)c2cc(N(c3ccccc3)c3ccc4ccccc4c3)ccc2-c2sc3c(c21)C(C)(C)c1cc(N(c2ccccc2)c2ccccc2-c2ccccc2)ccc1-3 ; CC1(C)c2cc(N(c3ccccc3)c3ccc4c(ccc5ccccc54)c3)ccc2-c2sc3c(c21)C(C)(C)c1cc(N(c2ccccc2)c2ccccc2-c2ccccc2)ccc1-3]

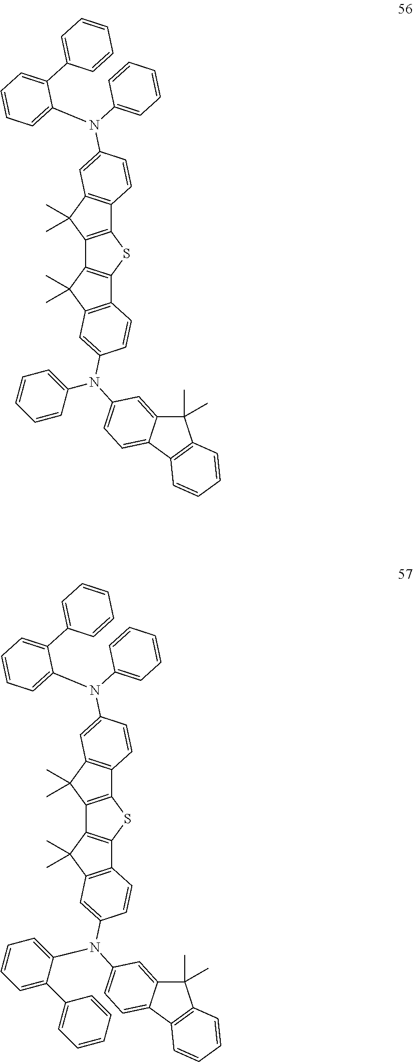 [SMILES: CC1(C)c2ccccc2-c2ccc(N(c3ccccc3)c3ccc4c(c3)C(C)(C)c3c-4sc4c3C(C)(C)c3cc(N(c5ccccc5)c5ccccc5-c5ccccc5)ccc3-4)cc21 ; CC1(C)c2ccccc2-c2ccc(N(c3ccc4c(c3)C(C)(C)c3c-4sc4c3C(C)(C)c3cc(N(c5ccccc5)c5ccccc5-c5ccccc5)ccc3-4)c3ccccc3-c3ccccc3)cc21]

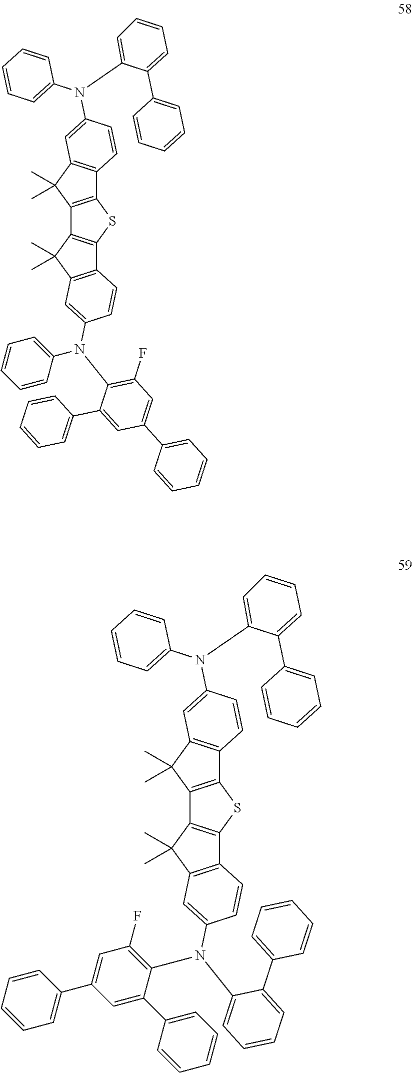 [SMILES: CC1(C)c2cc(N(c3ccccc3)c3ccccc3-c3ccccc3)ccc2-c2sc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccccc3-c3ccccc3)ccc2-c2sc3c(c21)C(C)(C)c1cc(N(c2ccccc2-c2ccccc2)c2c(F)cc(-c4ccccc4)cc2-c2ccccc2)ccc1-3]

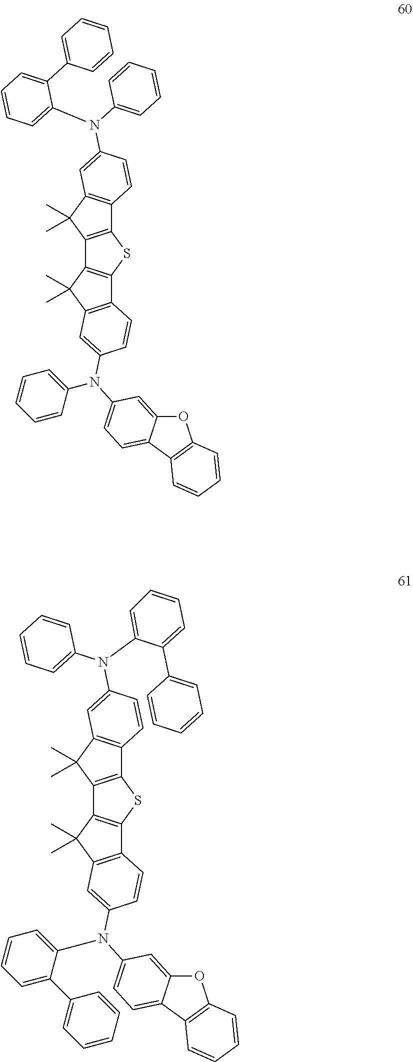 [SMILES: CC1(C)c2cc(N(c3ccccc3)c3ccc4c(c3)oc3ccccc34)ccc2-c2sc3c(c21)C(C)(C)c1cc(N(c2ccccc2)c2ccccc2-c2ccccc2)ccc1-3 ; CC1(C)c2cc(N(c3ccccc3)c3ccccc3-c3ccccc3)ccc2-c2sc3c(c21)C(C)(C)c1cc(N(c2ccc4c(c2)oc2ccccc24)c2ccccc2-c2ccccc2)ccc1-3]

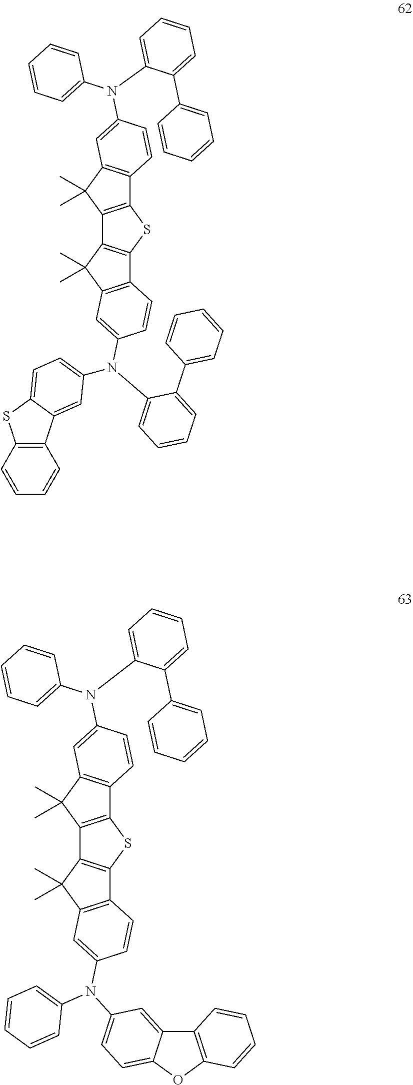 [SMILES: CC1(C)c2cc(N(c3ccccc3)c3ccccc3-c3ccccc3)ccc2-c2sc3c(c21)C(C)(C)c1cc(N(c2ccc4sc5ccccc5c4c2)c2ccccc2-c2ccccc2)ccc1-3 ; CC1(C)c2cc(N(c3ccccc3)c3ccc4oc5ccccc5c4c3)ccc2-c2sc3c(c21)C(C)(C)c1cc(N(c2ccccc2)c2ccccc2-c2ccccc2)ccc1-3]

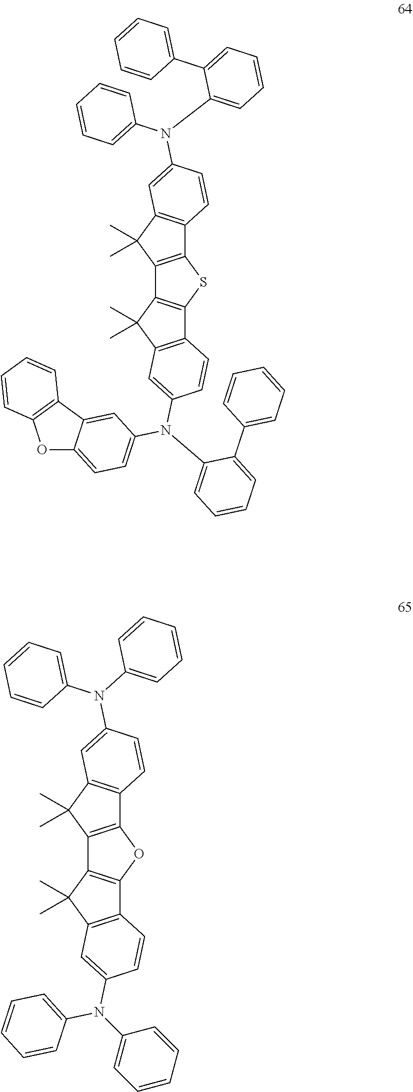 [SMILES: CC1(C)c2cc(N(c3ccccc3)c3ccccc3-c3ccccc3)ccc2-c2sc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2oc3c(c21)C(C)(C)c1cc(N(c2ccccc2)c2ccccc2)ccc1-3]

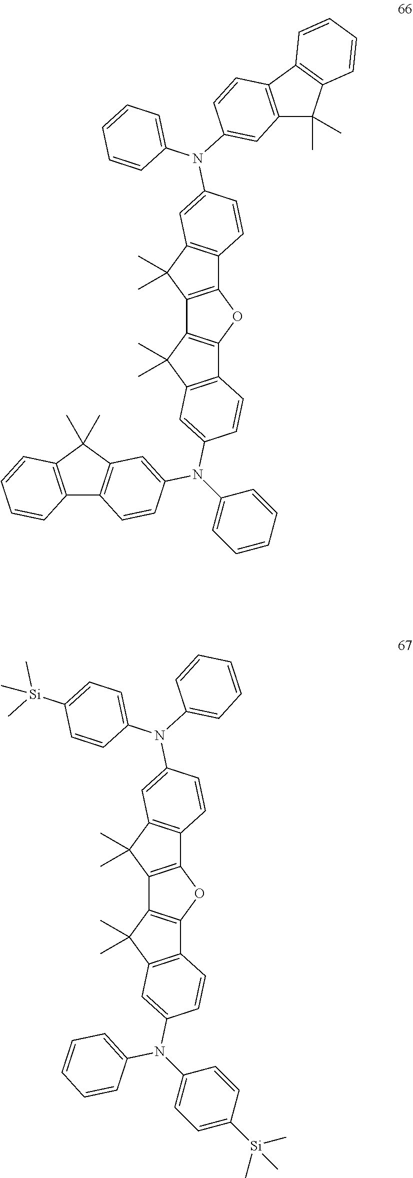

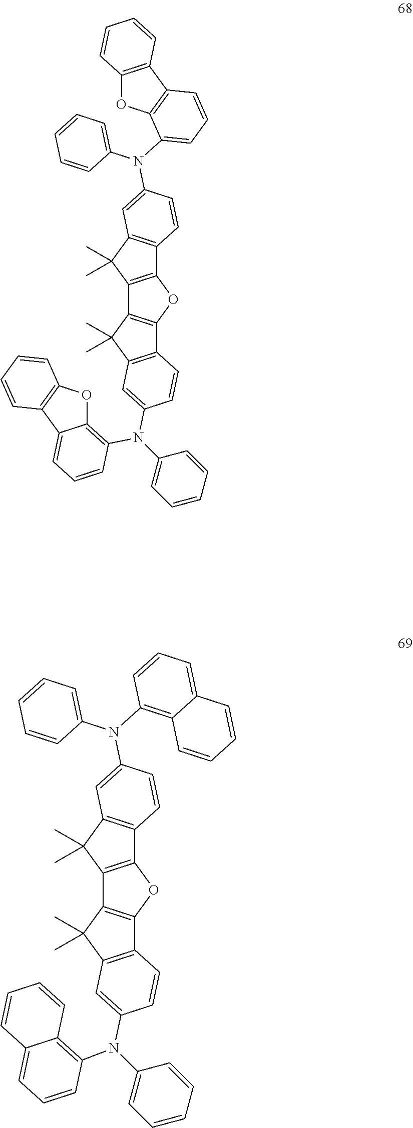 [SMILES: CC1(C)c2cc(N(c3ccccc3)c3cccc4c3oc3ccccc34)ccc2-c2oc3c(c21)C(C)(C)c1cc(N(c2ccccc2)c2cccc4c2oc2ccccc24)ccc1-3 ; CC1(C)c2cc(N(c3ccccc3)c3cccc4ccccc34)ccc2-c2oc3c(c21)C(C)(C)c1cc(N(c2ccccc2)c2cccc4ccccc24)ccc1-3]

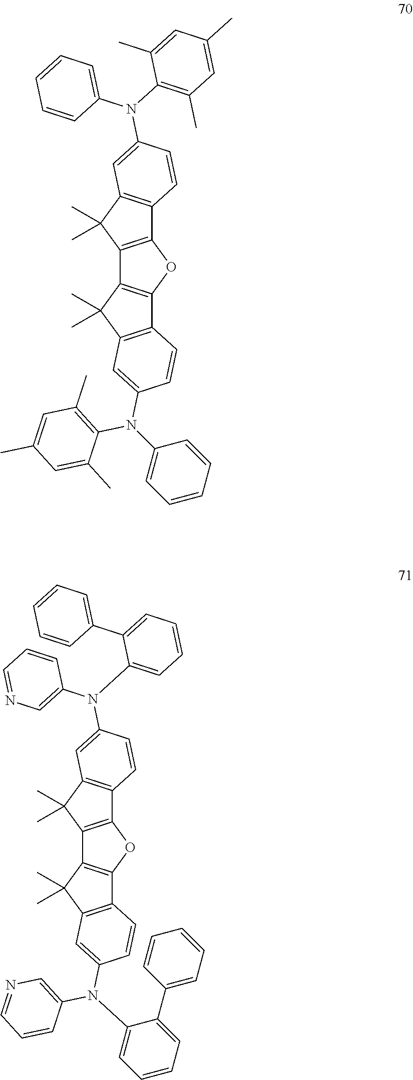

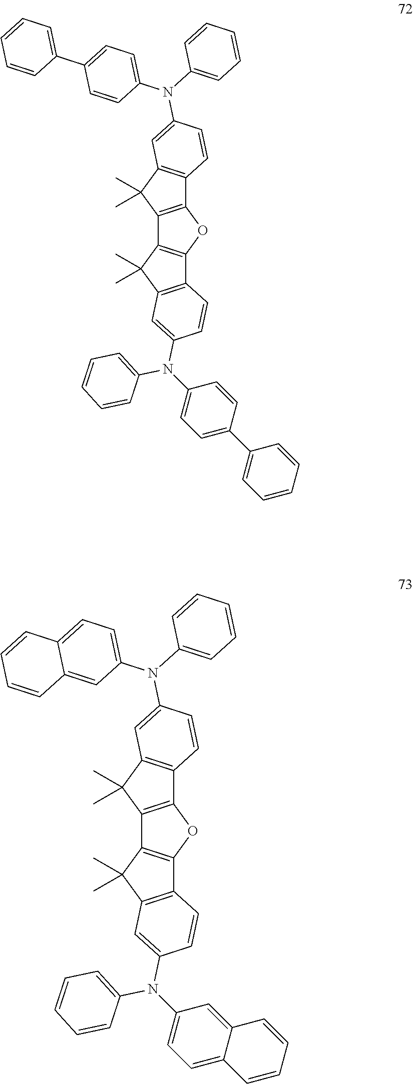 [SMILES: CC1(C)c2cc(N(c3ccccc3)c3ccc(-c4ccccc4)cc3)ccc2-c2oc3c(c21)C(C)(C)c1cc(N(c2ccccc2)c2ccc(-c4ccccc4)cc2)ccc1-3 ; CC1(C)c2cc(N(c3ccccc3)c3ccc4ccccc4c3)ccc2-c2oc3c(c21)C(C)(C)c1cc(N(c2ccccc2)c2ccc4ccccc4c2)ccc1-3]

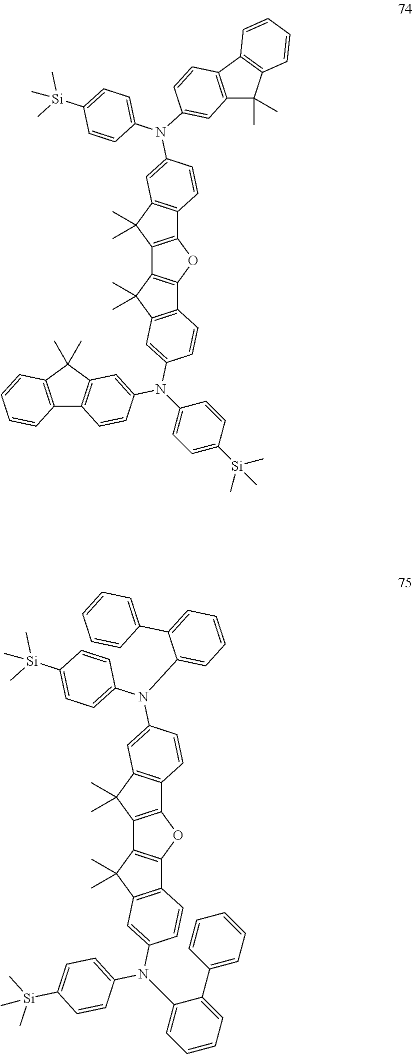 [SMILES: CC1(C)c2ccccc2-c2ccc(N(c3ccc([Si](C)(C)C)cc3)c3ccc4c(c3)C(C)(C)c3c-4oc4c3C(C)(C)c3cc(N(c5ccc([Si](C)(C)C)cc5)c5ccc6c(c5)C(C)(C)c5ccccc5-6)ccc3-4)cc21 ; CC1(C)c2cc(N(c3ccc([Si](C)(C)C)cc3)c3ccccc3-c3ccccc3)ccc2-c2oc3c(c21)C(C)(C)c1cc(N(c2ccc([Si](C)(C)C)cc2)c2ccccc2-c2ccccc2)ccc1-3]

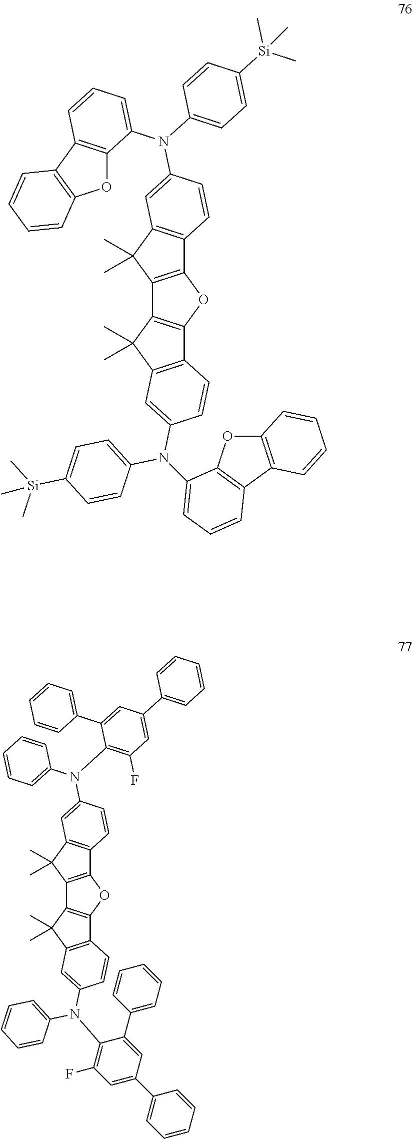 [SMILES: CC1(C)c2cc(N(c3ccc([Si](C)(C)C)cc3)c3cccc4c3oc3ccccc34)ccc2-c2oc3c(c21)C(C)(C)c1cc(N(c2ccc([Si](C)(C)C)cc2)c2cccc4c2oc2ccccc24)ccc1-3 ; CC1(C)c2cc(N(c3ccccc3)c3c(F)cc(-c4ccccc4)cc3-c3ccccc3)ccc2-c2oc3c(c21)C(C)(C)c1cc(N(c2ccccc2)c2c(F)cc(-c4ccccc4)cc2-c2ccccc2)ccc1-3]

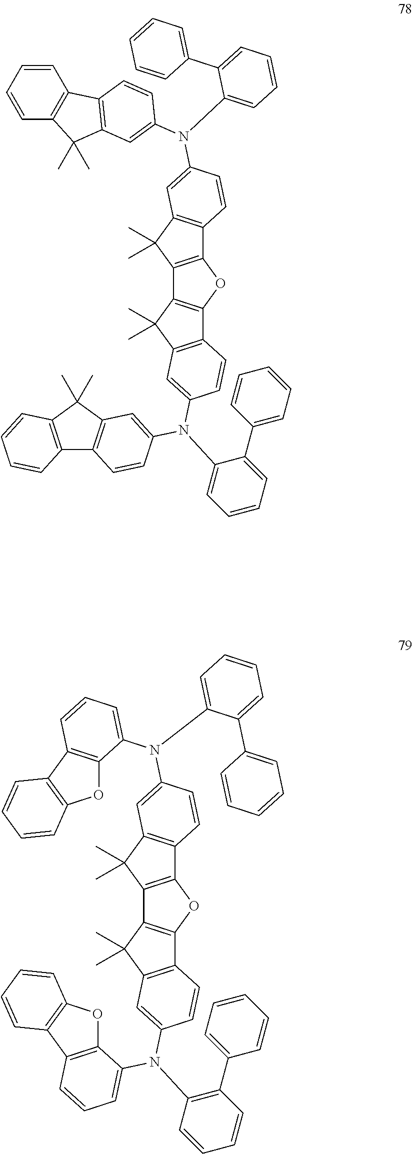 [SMILES: CC1(C)c2ccccc2-c2ccc(N(c3ccc4c(c3)C(C)(C)c3c-4oc4c3C(C)(C)c3cc(N(c5ccc6c(c5)C(C)(C)c5ccccc5-6)c5ccccc5-c5ccccc5)ccc3-4)c3ccccc3-c3ccccc3)cc21 ; CC1(C)c2cc(N(c3ccccc3-c3ccccc3)c3cccc4c3oc3ccccc34)ccc2-c2oc3c(c21)C(C)(C)c1cc(N(c2ccccc2-c2ccccc2)c2cccc4c2oc2ccccc24)ccc1-3]

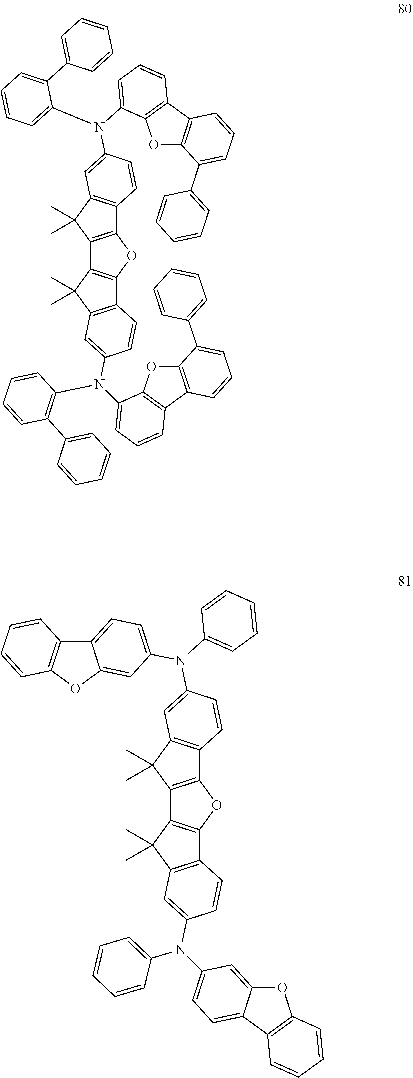 [SMILES: CC1(C)c2cc(N(c3ccccc3-c3ccccc3)c3cccc4c3oc3c(-c5ccccc5)cccc34)ccc2-c2oc3c(c21)C(C)(C)c1cc(N(c2ccccc2-c2ccccc2)c2cccc4c2oc2c(-c5ccccc5)cccc24)ccc1-3 ; CC1(C)c2cc(N(c3ccccc3)c3ccc4c(c3)oc3ccccc34)ccc2-c2oc3c(c21)C(C)(C)c1cc(N(c2ccccc2)c2ccc4c(c2)oc2ccccc24)ccc1-3]

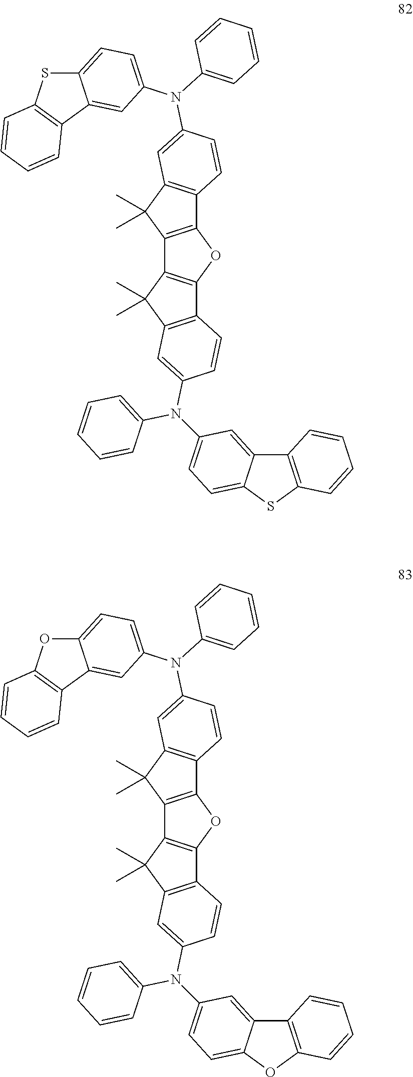 [SMILES: CC1(C)c2cc(N(c3ccccc3)c3ccc4sc5ccccc5c4c3)ccc2-c2oc3c(c21)C(C)(C)c1cc(N(c2ccccc2)c2ccc4sc5ccccc5c4c2)ccc1-3 ; CC1(C)c2cc(N(c3ccccc3)c3ccc4oc5ccccc5c4c3)ccc2-c2oc3c(c21)C(C)(C)c1cc(N(c2ccccc2)c2ccc4oc5ccccc5c4c2)ccc1-3]

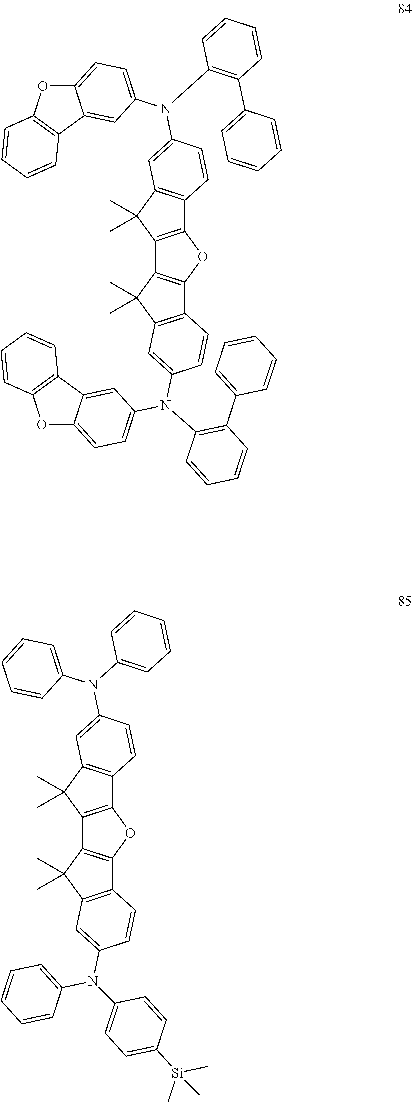 [SMILES: CC1(C)c2cc(N(c3ccc4oc5ccccc5c4c3)c3ccccc3-c3ccccc3)ccc2-c2oc3c(c21)C(C)(C)c1cc(N(c2ccc4oc5ccccc5c4c2)c2ccccc2-c2ccccc2)ccc1-3 ; CC1(C)c2cc(N(c3ccccc3)c3ccccc3)ccc2-c2oc3c(c21)C(C)(C)c1cc(N(c2ccccc2)c2ccc([Si](C)(C)C)cc2)ccc1-3]

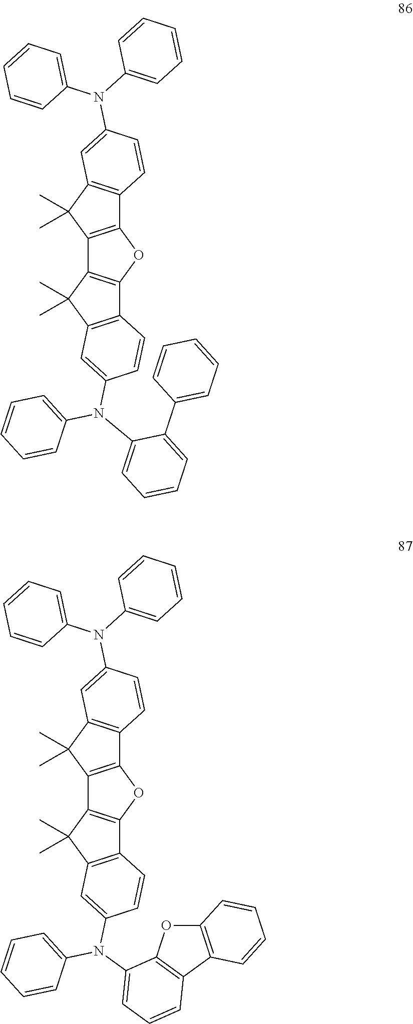 [SMILES: CC1(C)c2cc(N(c3ccccc3)c3ccccc3)ccc2-c2oc3c(c21)C(C)(C)c1cc(N(c2ccccc2)c2ccccc2-c2ccccc2)ccc1-3 ; CC1(C)c2cc(N(c3ccccc3)c3ccccc3)ccc2-c2oc3c(c21)C(C)(C)c1cc(N(c2ccccc2)c2cccc4c2oc2ccccc24)ccc1-3]

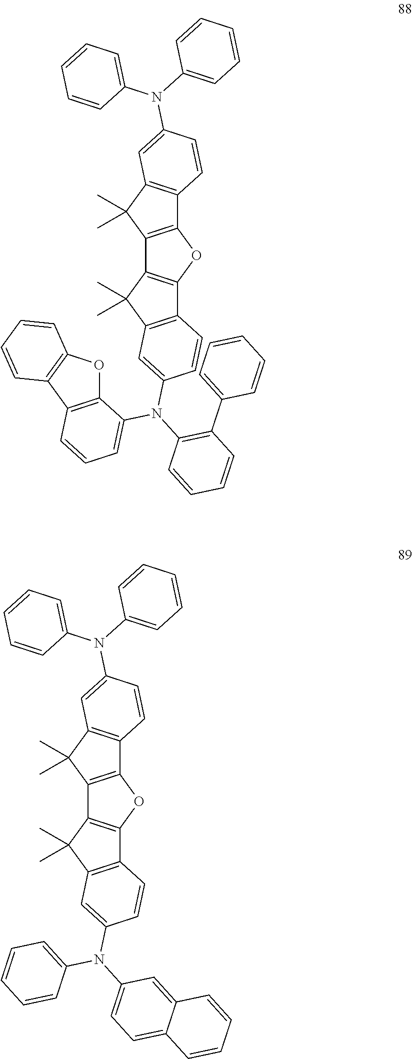 [SMILES: CC1(C)c2cc(N(c3ccccc3)c3ccccc3)ccc2-c2oc3c(c21)C(C)(C)c1cc(N(c2ccccc2-c2ccccc2)c2cccc4c2oc2ccccc24)ccc1-3 ; CC1(C)c2cc(N(c3ccccc3)c3ccccc3)ccc2-c2oc3c(c21)C(C)(C)c1cc(N(c2ccccc2)c2ccc4ccccc4c2)ccc1-3]

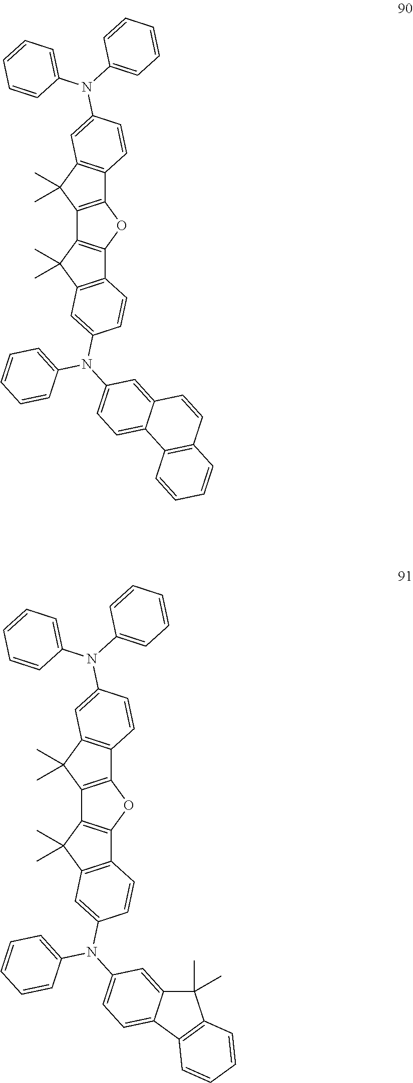 [SMILES: CC1(C)c2cc(N(c3ccccc3)c3ccccc3)ccc2-c2oc3c(c21)C(C)(C)c1cc(N(c2ccccc2)c2ccc4c(ccc5ccccc54)c2)ccc1-3 ; CC1(C)c2ccccc2-c2ccc(N(c3ccccc3)c3ccc4c(c3)C(C)(C)c3c-4oc4c3C(C)(C)c3cc(N(c5ccccc5)c5ccccc5)ccc3-4)cc21]

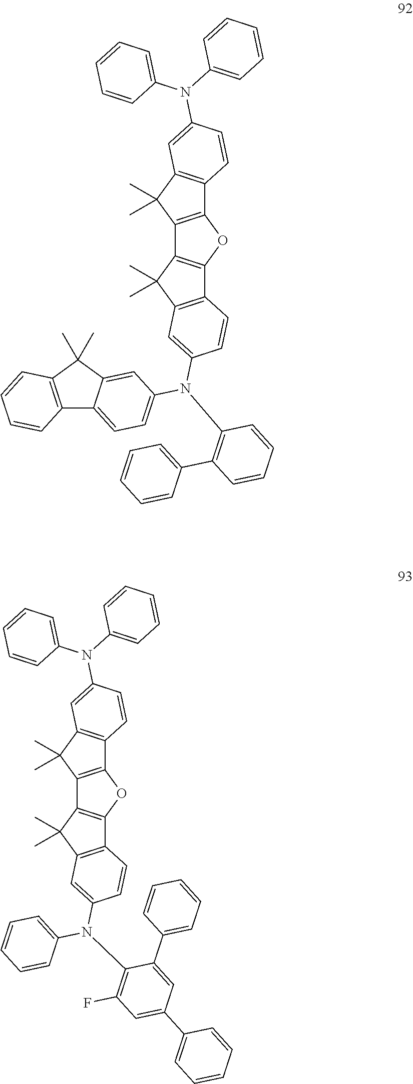 [SMILES: CC1(C)c2ccccc2-c2ccc(N(c3ccc4c(c3)C(C)(C)c3c-4oc4c3C(C)(C)c3cc(N(c5ccccc5)c5ccccc5)ccc3-4)c3ccccc3-c3ccccc3)cc21 ; CC1(C)c2cc(N(c3ccccc3)c3ccccc3)ccc2-c2oc3c(c21)C(C)(C)c1cc(N(c2ccccc2)c2c(F)cc(-c4ccccc4)cc2-c2ccccc2)ccc1-3]

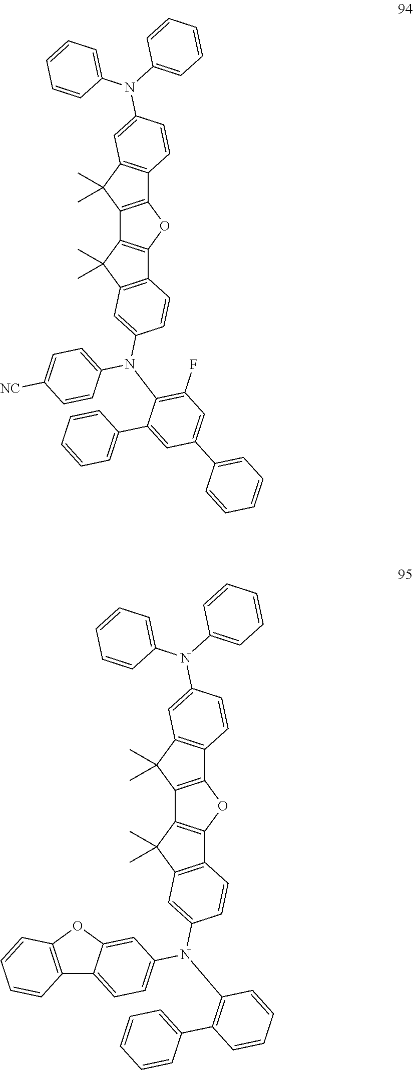 [SMILES: CC1(C)c2cc(N(c3ccccc3)c3ccccc3)ccc2-c2oc3c(c21)C(C)(C)c1cc(N(c2ccc(C#N)cc2)c2c(F)cc(-c4ccccc4)cc2-c2ccccc2)ccc1-3 ; CC1(C)c2cc(N(c3ccccc3)c3ccccc3)ccc2-c2oc3c(c21)C(C)(C)c1cc(N(c2ccc4c(c2)oc2ccccc24)c2ccccc2-c2ccccc2)ccc1-3]

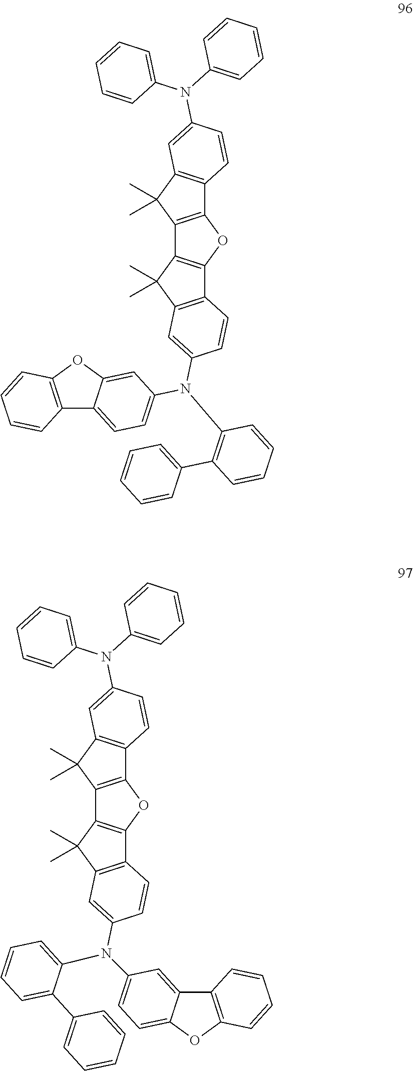 [SMILES: CC1(C)c2cc(N(c3ccccc3)c3ccccc3)ccc2-c2oc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccccc3)ccc2-c2oc3c(c21)C(C)(C)c1cc(N(c2ccc4oc5ccccc5c4c2)c2ccccc2-c2ccccc2)ccc1-3]

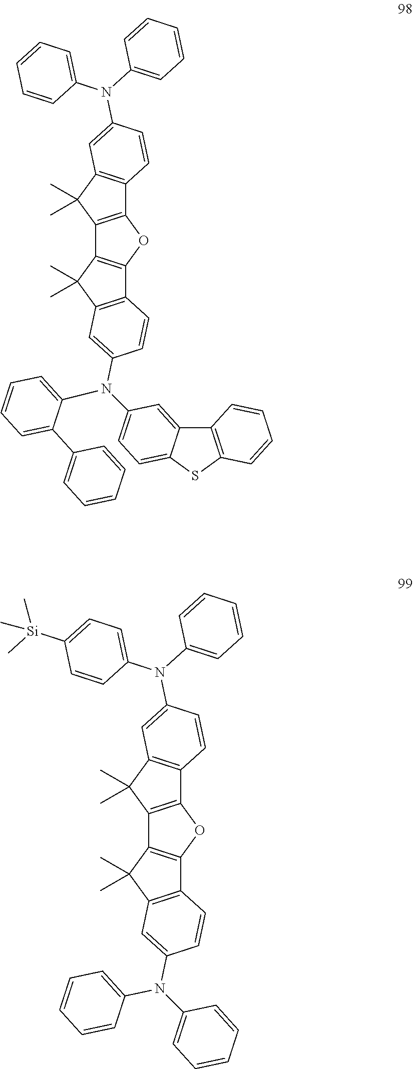 [SMILES: CC1(C)c2cc(N(c3ccccc3)c3ccccc3)ccc2-c2oc3c(c21)C(C)(C)c1cc(N(c2ccc4sc5ccccc5c4c2)c2ccccc2-c2ccccc2)ccc1-3 ; CC1(C)c2cc(N(c3ccccc3)c3ccccc3)ccc2-c2oc3c(c21)C(C)(C)c1cc(N(c2ccccc2)c2ccc([Si](C)(C)C)cc2)ccc1-3]

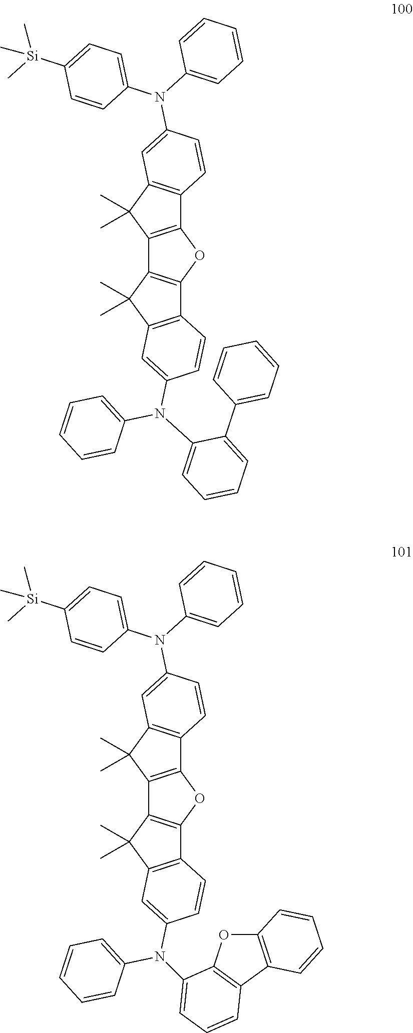 [SMILES: CC1(C)c2cc(N(c3ccccc3)c3ccc([Si](C)(C)C)cc3)ccc2-c2oc3c(c21)C(C)(C)c1cc(N(c2ccccc2)c2ccccc2-c2ccccc2)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2cccc4c2oc2ccccc24)ccc1-3]

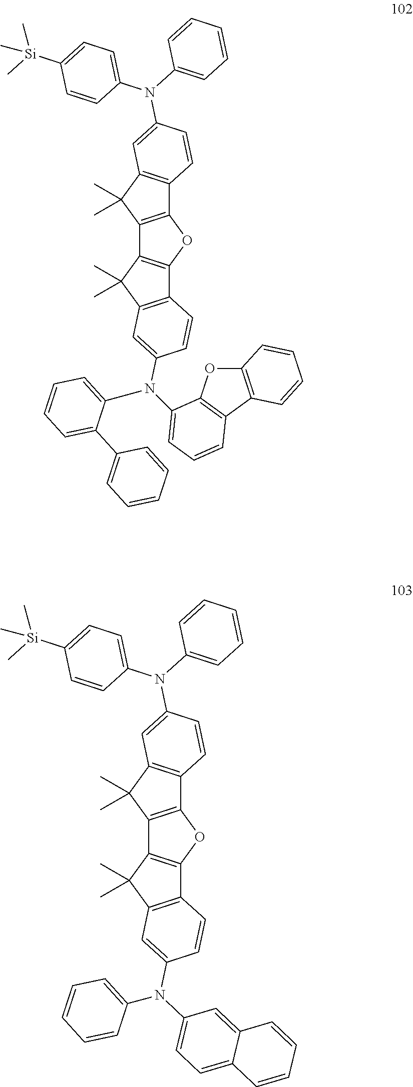 [SMILES: CC1(C)c2cc(N(c3ccccc3)c3ccc([Si](C)(C)C)cc3)ccc2-c2oc3c(c21)C(C)(C)c1cc(N(c2ccccc2-c2ccccc2)c2cccc4c2oc2ccccc24)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2ccc4ccccc4c2)ccc1-3]

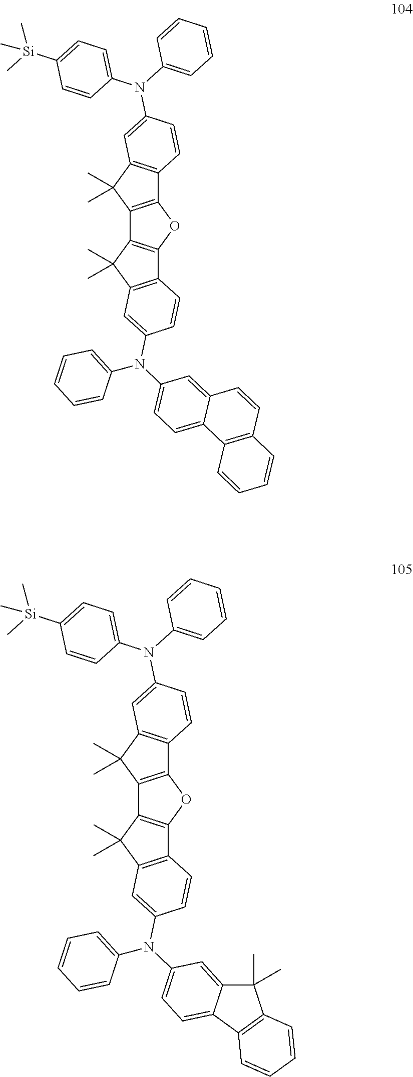 [SMILES: CC1(C)c2cc(N(c3ccccc3)c3ccc([Si](C)(C)C)cc3)ccc2-c2oc3c(c21)C(C)(C)c1cc(N(c2ccccc2)c2ccc4c(ccc5ccccc54)c2)ccc1-3 ; CC1(C)c2ccccc2-c2ccc(N(c3ccccc3)c3ccc4c(c3)C(C)(C)c3c-4oc4c3C(C)(C)c3cc(N(c5ccccc5)c5ccc([Si](C)(C)C)cc5)ccc3-4)cc21]

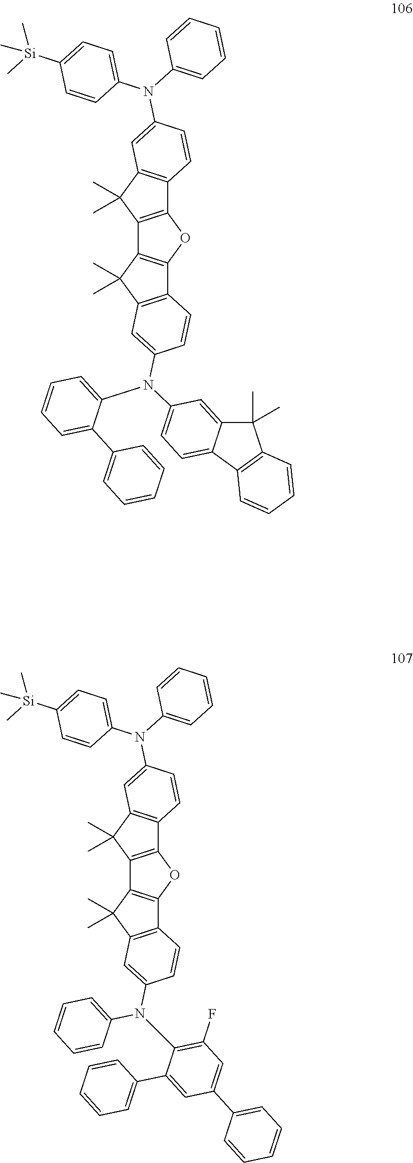 [SMILES: CC1(C)c2ccccc2-c2ccc(N(c3ccc4c(c3)C(C)(C)c3c-4oc4c3C(C)(C)c3cc(N(c5ccccc5)c5ccc([Si](C)(C)C)cc5)ccc3-4)c3ccccc3-c3ccccc3)cc21 ; CC1(C)c2cc(N(c3ccccc3)c3ccc([Si](C)(C)C)cc3)ccc2-c2oc3c(c21)C(C)(C)c1cc(N(c2ccccc2)c2c(F)cc(-c4ccccc4)cc2-c2ccccc2)ccc1-3]

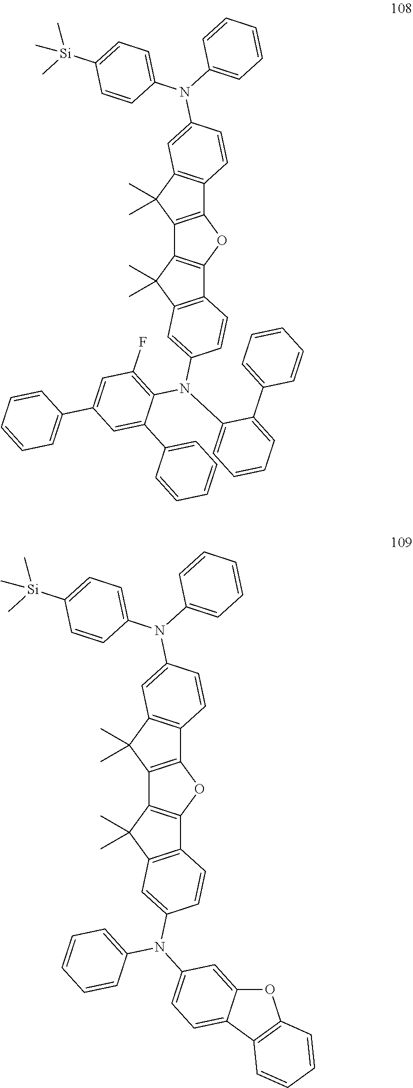 [SMILES: CC1(C)c2cc(N(c3ccccc3)c3ccc([Si](C)(C)C)cc3)ccc2-c2oc3c(c21)C(C)(C)c1cc(N(c2ccccc2-c2ccccc2)c2c(F)cc(-c4ccccc4)cc2-c2ccccc2)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2ccc4c(c2)oc2ccccc24)ccc1-3]

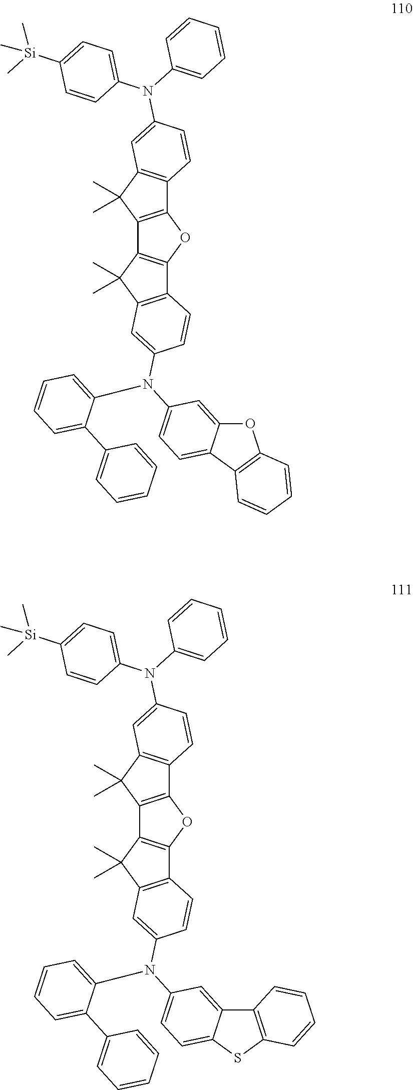 [SMILES: CC1(C)c2cc(N(c3ccccc3)c3ccc([Si](C)(C)C)cc3)ccc2-c2oc3c(c21)C(C)(C)c1cc(N(c2ccc4c(c2)oc2ccccc24)c2ccccc2-c2ccccc2)ccc1-3 ; CC1(C)c2cc(N(c3ccccc3)c3ccc([Si](C)(C)C)cc3)ccc2-c2oc3c(c21)C(C)(C)c1cc(N(c2ccc4sc5ccccc5c4c2)c2ccccc2-c2ccccc2)ccc1-3]

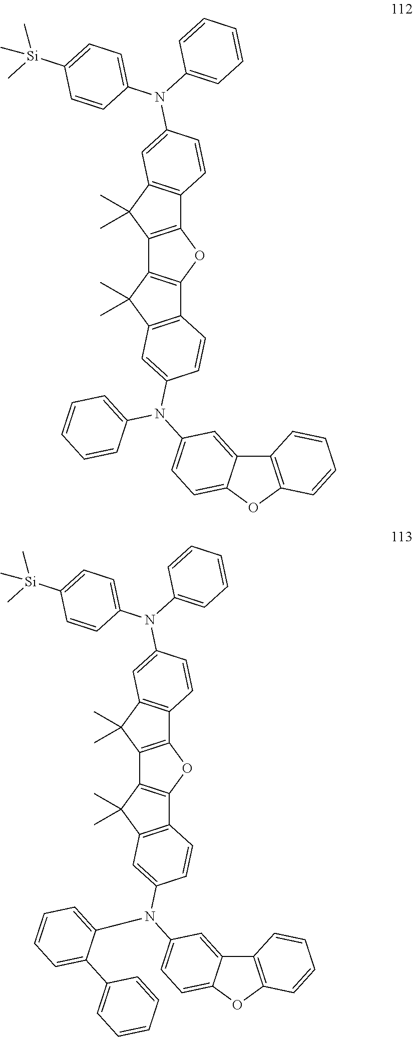 [SMILES: CC1(C)c2cc(N(c3ccccc3)c3ccc([Si](C)(C)C)cc3)ccc2-c2oc3c(c21)C(C)(C)c1cc(N(c2ccccc2)c2ccc4oc5ccccc5c4c2)ccc1-3 ; CC1(C)c2cc(N(c3ccccc3)c3ccc([Si](C)(C)C)cc3)ccc2-c2oc3c(c21)C(C)(C)c1cc(N(c2ccc4oc5ccccc5c4c2)c2ccccc2-c2ccccc2)ccc1-3]

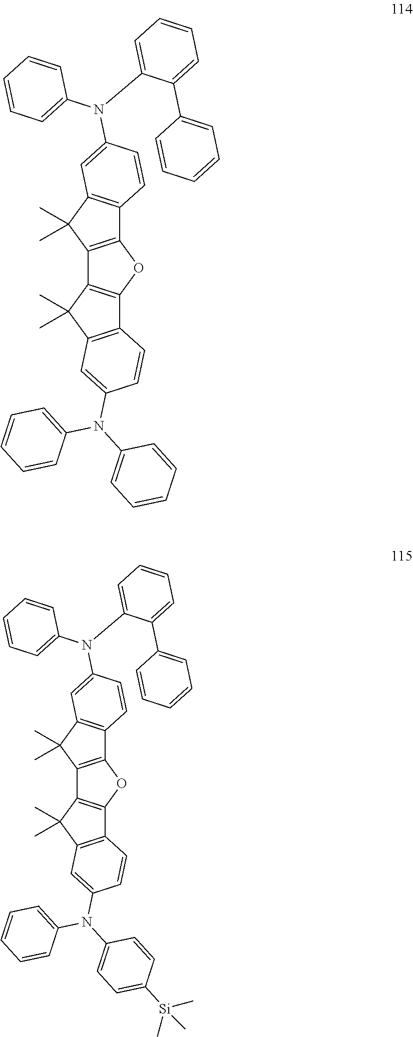 [SMILES: CC1(C)c2cc(N(c3ccccc3)c3ccccc3)ccc2-c2oc3c(c21)C(C)(C)c1cc(N(c2ccccc2)c2ccccc2-c2ccccc2)ccc1-3 ; CC1(C)c2cc(N(c3ccccc3)c3ccc([Si](C)(C)C)cc3)ccc2-c2oc3c(c21)C(C)(C)c1cc(N(c2ccccc2)c2ccccc2-c2ccccc2)ccc1-3]

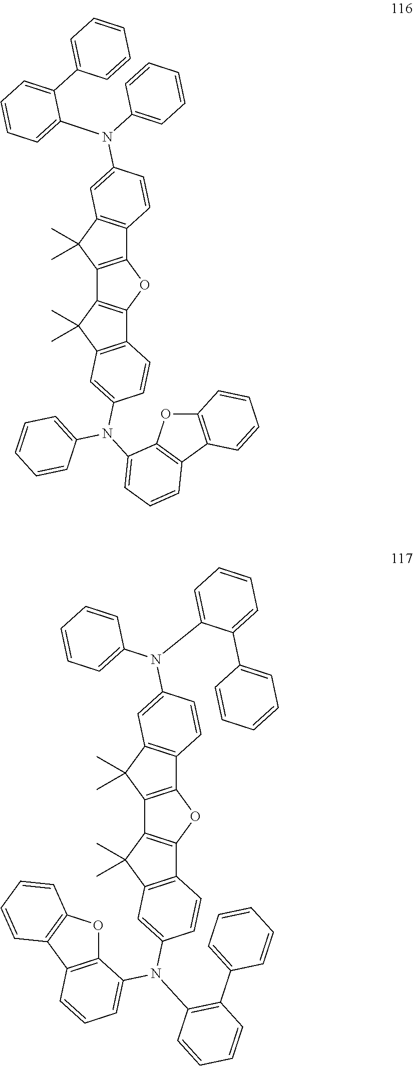 [SMILES: CC1(C)c2cc(N(c3ccccc3)c3ccccc3-c3ccccc3)ccc2-c2oc3c(c21)C(C)(C)c1cc(N(c2ccccc2)c2cccc4c2oc2ccccc24)ccc1-3 ; CC1(C)c2cc(N(c3ccccc3)c3ccccc3-c3ccccc3)ccc2-c2oc3c(c21)C(C)(C)c1cc(N(c2ccccc2-c2ccccc2)c2cccc4c2oc2ccccc24)ccc1-3]

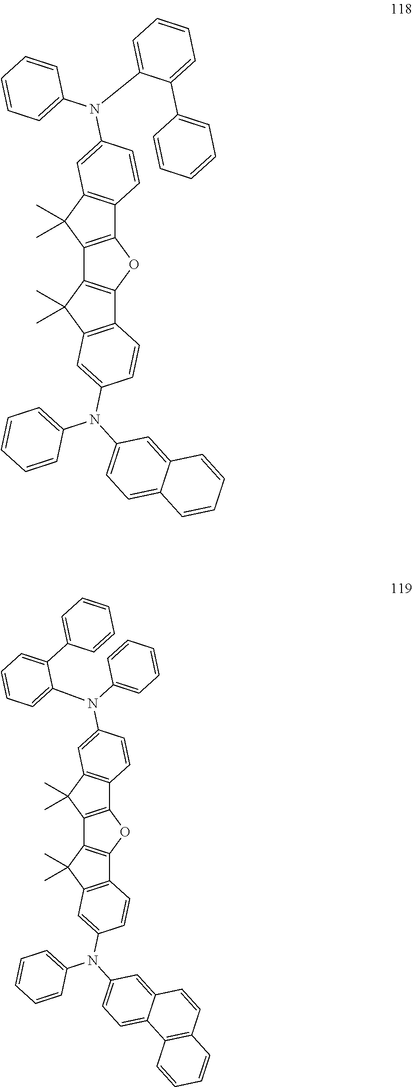 [SMILES: CC1(C)c2cc(N(c3ccccc3)c3ccc4ccccc4c3)ccc2-c2oc3c(c21)C(C)(C)c1cc(N(c2ccccc2)c2ccccc2-c2ccccc2)ccc1-3 ; CC1(C)c2cc(N(c3ccccc3)c3ccc4c(ccc5ccccc54)c3)ccc2-c2oc3c(c21)C(C)(C)c1cc(N(c2ccccc2)c2ccccc2-c2ccccc2)ccc1-3]

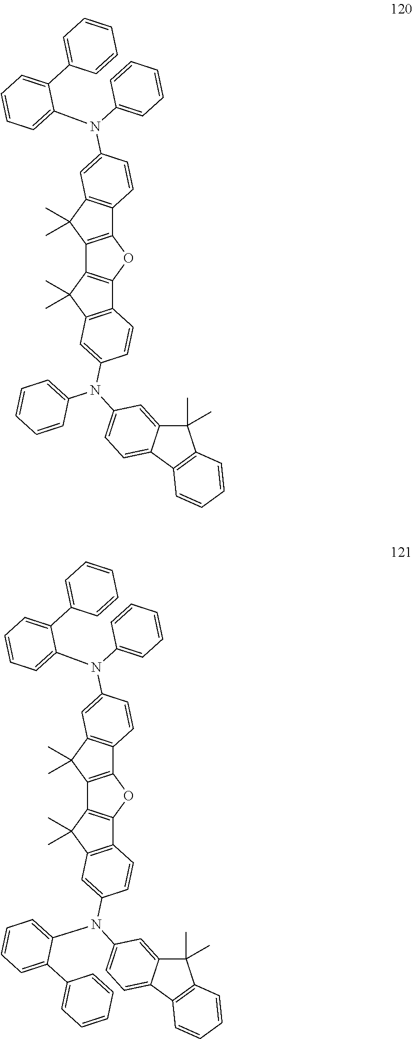 [SMILES: CC1(C)c2ccccc2-c2ccc(N(c3ccccc3)c3ccc4c(c3)C(C)(C)c3c-4oc4c3C(C)(C)c3cc(N(c5ccccc5)c5ccccc5-c5ccccc5)ccc3-4)cc21 ; CC1(C)c2ccccc2-c2ccc(N(c3ccc4c(c3)C(C)(C)c3c-4oc4c3C(C)(C)c3cc(N(c5ccccc5)c5ccccc5-c5ccccc5)ccc3-4)c3ccccc3-c3ccccc3)cc21]

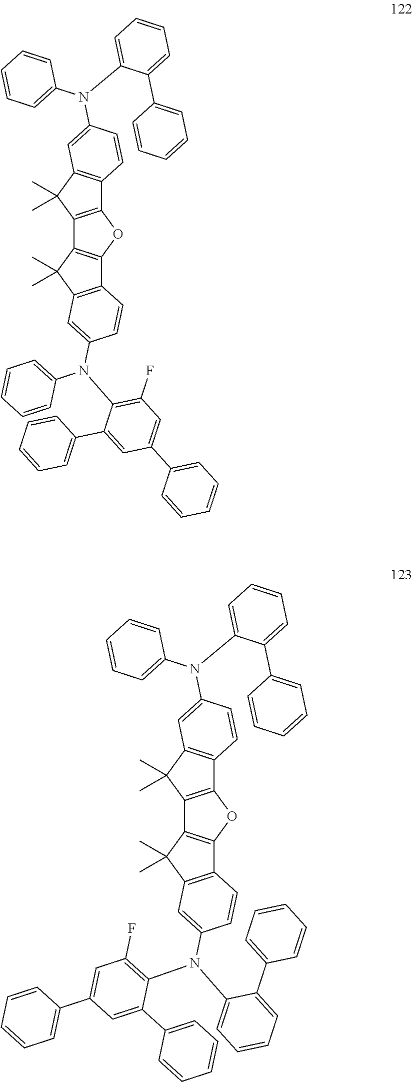 [SMILES: CC1(C)c2cc(N(c3ccccc3)c3ccccc3-c3ccccc3)ccc2-c2oc3c(c21)C(C)(C)c1cc(N(c2ccccc2)c2c(F)cc(-c4ccccc4)cc2-c2ccccc2)ccc1-3 ; CC1(C)c2cc(N(c3ccccc3)c3ccccc3-c3ccccc3)ccc2-c2oc3c(c21)C(C)(C)c1cc(N(c2ccccc2-c2ccccc2)c2c(F)cc(-c4ccccc4)cc2-c2ccccc2)ccc1-3]

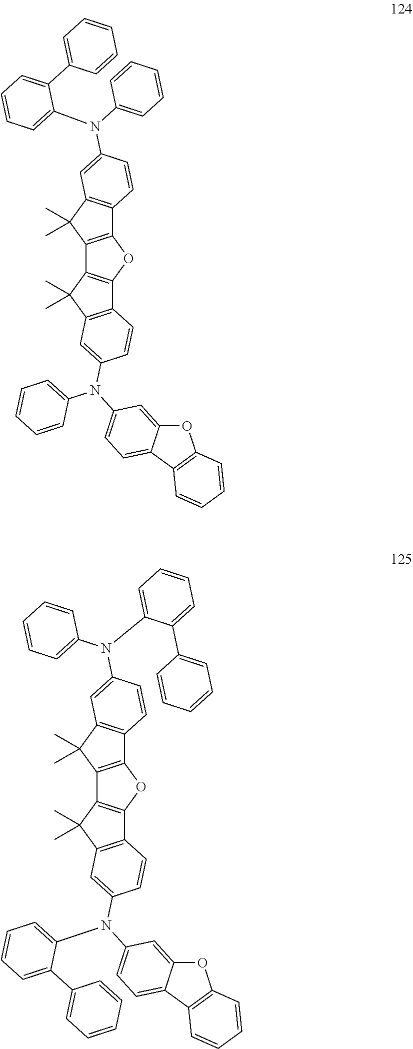 [SMILES: CC1(C)c2cc(N(c3ccccc3)c3ccc4c(c3)oc3ccccc34)ccc2-c2oc3c(c21)C(C)(C)c1cc(N(c2ccccc2)c2ccccc2-c2ccccc2)ccc1-3 ; CC1(C)c2cc(N(c3ccccc3)c3ccccc3-c3ccccc3)ccc2-c2oc3c(c21)C(C)(C)c1cc(N(c2ccc4c(c2)oc2ccccc24)c2ccccc2-c2ccccc2)ccc1-3]

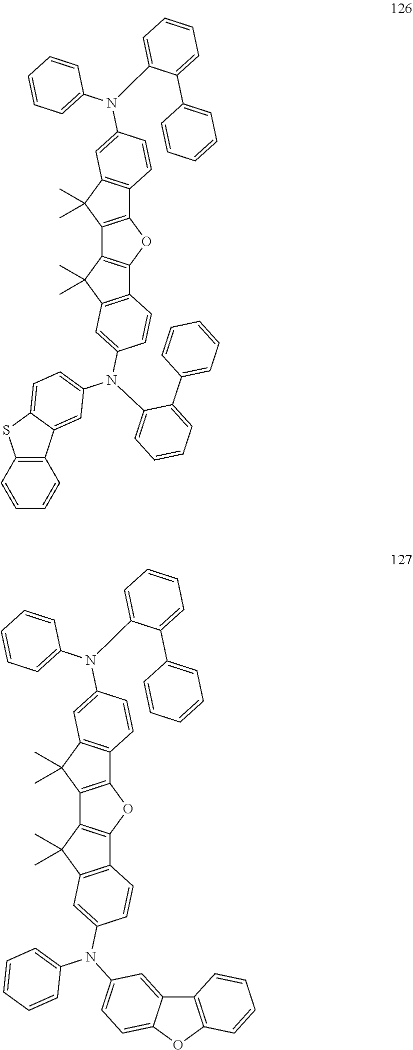 [SMILES: CC1(C)c2cc(N(c3ccccc3)c3ccccc3-c3ccccc3)ccc2-c2oc3c(c21)C(C)(C)c1cc(N(c2ccc4sc5ccccc5c4c2)c2ccccc2-c2ccccc2)ccc1-3 ; CC1(C)c2cc(N(c3ccccc3)c3ccc4oc5ccccc5c4c3)ccc2-c2oc3c(c21)C(C)(C)c1cc(N(c2ccccc2)c2ccccc2-c2ccccc2)ccc1-3]

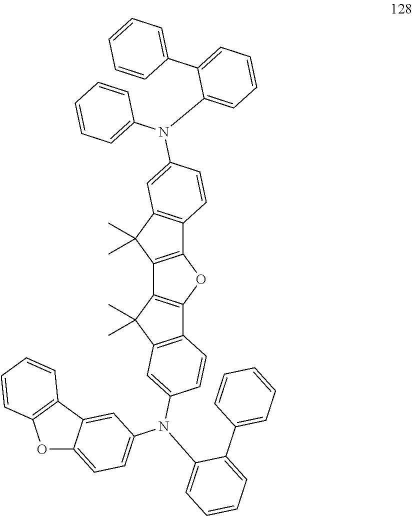 [SMILES: CC1(C)c2cc(N(c3ccccc3)c3ccccc3-c3ccccc3)ccc2-c2oc3c(c21)C(C)(C)c1cc(N(c2ccc4oc5ccccc5c4c2)c2ccccc2-c2ccccc2)ccc1-3]

9. An organic light-emitting device, comprising:
a first electrode;
a second electrode facing the first electrode; and
an organic layer between the first electrode and the second electrode, wherein the organic layer comprises an emission layer and the compound of claim 1.

10. The organic light-emitting device of claim 9, wherein the organic layer is formed by a wet coating method.

11. The organic light-emitting device of claim 9, wherein the first electrode is an anode,
the second electrode is a cathode, and
the organic layer comprises i) a hole transport region between the first electrode and the emission layer and comprising at least one selected from a hole injection layer, a hole transport layer, and an electron blocking layer, and ii) an electron transport region between the emission layer and the second electrode and comprising at least one selected from a hole blocking layer, an electron transport layer, and an electron injection layer.

12. The organic light-emitting device of claim 11, wherein the emission layer comprises the compound of claim 1.

13. The organic light-emitting device of claim 11, wherein the emission layer comprises the compound of claim 1 as a dopant.

14. The organic light-emitting device of claim 11, wherein the hole transport region comprises a charge-generating material.

15. The organic light-emitting device of claim 14, wherein the charge-generating material is a p-dopant.

16. The organic light-emitting device of claim 15, wherein the p-dopant is selected from a quinone derivative, a metal oxide, and a cyano group-containing compound.

17. The organic light-emitting device of claim 11, wherein the electron transport region comprises a metal complex.

18. The organic light-emitting device of claim 11, wherein the electron transport region comprises a Li complex.

19. The organic light-emitting device of claim 11, wherein the electron transport region comprises ET-D1 or ET-D2 below:

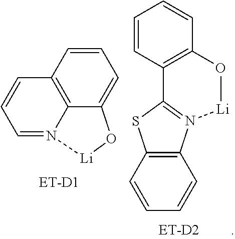

20. A display apparatus comprising the organic light-emitting device of claim 9, wherein the first electrode of the organic light-emitting device is electrically coupled to source and drain electrodes of a thin film transistor.
